(19) United States
(12) Patent Application Publication
(10) Pub. No.: US 2012/0115842 A1
(43) Pub. Date: May 10, 2012

(54) Substituted Oxindole Derivatives, Medicaments Containing Said Derivatives and Use Thereof

(76) Inventors: Wilfried Lubisch, Heidelberg (DE); Thorsten Oost, Biberach an der Riss (DE); Liliane Unger, Ludwigshafen (DE); Wilfried Hornberger, Neustadt (DE); Herve Geneste, Neuhofen (DE)

(21) Appl. No.: 11/794,582

(22) PCT Filed: Dec. 30, 2005
(86) PCT No.: PCT/EP2005/014150

§ 371 Date: Nov. 28, 2009

Related U.S. Application Data
(60) Provisional application No. 60/663,349, filed on Mar. 18, 2005.

(30) Foreign Application Priority Data
Dec. 31, 2004 (DE) 102004063843.8
Mar. 18, 2005 (DE) 102005014105.6

(51) Int. Cl. A61K 31/404; C07D 401/04; A61K 31/454; C07D 209/42; C07D 413/04; A61K 31/5377; A61P 3/10; A61P 13/00; A61P 7/00; A61P 9/12; A61P 9/04; A61P 9/10; A61P 13/12; A61P 1/00; A61P 1/06; A61P 1/16; A61P 1/04; A61P 1/08; A61P 25/22; A61P 25/28; A61P 25/18; A61P 25/00; A61P 9/00; C07D 403/04

(52) U.S. Cl. 514/210.18; 548/466; 514/414; 546/201; 514/323; 548/486; 514/418; 544/144; 514/235.2

(57) ABSTRACT

The invention relates to novel oxindole derivatives of general formula (I), wherein substituents A, B, X and Y are defined as in claim 1, medicaments containing said derivatives, and the use thereof in the prophylaxis and/or treatment of vasopressin-dependent and/or oxytocin-dependent diseases.

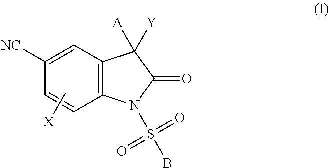

The present invention relates to novel 5-cyano-substituted oxindole derivatives, medicaments comprising them and to their use for the treatment of diseases.

Vasopressin is an endogenous hormone which exerts widely diverse effects on organs and tissues. It is suspected that the vasopressin system is involved in various pathological states such as, for example, heart failure and high blood pressure. At present, three receptors (V1a, V1b or V3 and V2) via which vasopressin mediates its numerous effects are known. Antagonists of these receptors are therefore being investigated as possible novel therapeutic approaches to the treatment of diseases. (M. Thibonnier, Exp.Opin. Invest. Drugs 1998, 7(5), 729-740).

Oxytocin is a hormone which is produced in neurosecretory neurons of the hypothalamus and —bound to neurophysins—is transported to the posterior pituitary lobe and is stored there. Oxytocin stimulates contraction of the uterine muscles and of the myoepithelial cells of the mammary gland (ejection of milk); the contractility of the uterus is altered by estrogens (promoting effect) and progestogens (inhibiting effect). Oxytocin is broken down by the enzyme oxytocinase. Oxytocin is used in obstetrics (e.g. for the induction of labor, in the event of postpartum uterine atony) (quoted from: Roche Lexikon Medizin 5th edition).

The present application describes novel substituted oxindoles which have an arylsulfonyl group in position 1. 1-Phenylsulfonyl-1,3-dihydro-2H-indol-2-ones have previously been described as ligands of vasopressin receptors. WO 93/15051, WO95/18105, WO 98/25901, WO 01/55130, WO 01/55134, WO 01/164668 and WO 01/98295 describe derivatives derived from the oxindole structure and having arylsulfonyl groups in position 1. These compounds differ essentially in the substitution in position 3.

In particular, WO 93/15051 and WO 98/25901 describe 1-phenylsulfonyl-1,3-dihydro-2H-indol-2-ones, in which the oxindole structure is substituted in position 3 by two alkyl radicals which may likewise be a cycloalkyl radical (spiro inkage), as ligands of vasopressin receptors. As alternative, the spiro ing may comprise heteroatoms such as oxygen and nitrogen (optionally with substituents).

WO 95/18105 describes 1-phenylsulfonyl-1,3-dihydro-2H-indol-2-ones which have a nitrogen atom in position 3 as ligands of vasopressin receptors. In addition, radicals which may be alkyl, cycloalkyl, phenyl or benzyl radicals are bonded in position 3 (in each case optionally with substituents).

Other publications, for example WO 01/55130, describe compounds which have nitrogen-containing rings (e.g. proline, homoproline, morpholine, tetrahydroisoquinoline, dihydroindole; in each case optionally with substituents) which are linked via their nitrogen atom to position 3 of the oxindole structure but which are substituted by phenylsulfonyl or phenyl groups (optionally with substituents) both in position 1 and in position 3 on the oxindole ring.

WO 03/008407 describes 1-phenylsulfonyloxindoles in which pyridylpiperazines are linked via an oxycarbonyl group to the oxindole in position 3.

It is an object of the present invention to provide further compounds for the treatment or prophylaxis of various vasopressin-dependent or oxytocin-dependent diseases which have a high and selective activity.

The object is achieved by at least one compound of the general formula (I),

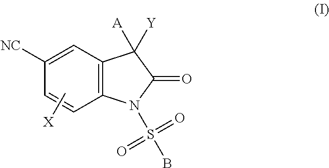

in which
A is C₆-C₁₀-aryl which may be substituted by one, two, three or four radicals selected from the group consisting of R_{A}¹, R_{A}², R_{A}³ and/or R_{A}⁴, in which R_{A}¹, R_{A}², R_{A}³ and R_{A}⁴ are independently of one another and independently of their respective occurrence selected from the group consisting of hydrogen, chlorine, bromine, iodine, fluorine, CN, OR_{A}⁵, COR_{A}⁵, COOR_{A}⁵, SR_{A}⁵, C₃-C₇-cycloalkyl, OCOR_{A}⁵, SO₂NR_{A}⁶R_{A}⁷, CONR_{A}⁶R_{A}⁷, C₀-C₄-alkylene-CN, C₁-C₆-haloalkyl, C₁-C₆-haloalkoxy, NO₂, C₀-C₄-alkylene-OR_{A}⁵, C₁-C₆-alkyl, C₂-C₆-alkenyl, C₂-C₆-alkynyl, C₂-C₆-alkenyloxy, C₂-C₆-alkynyloxy, C₁-C₄-alkylthio, C₀-C₄-alkylene-COR_{A}⁵, SO₂R_{A}⁵, C₀-C₄-alkylene-COOR_{A}⁵,
   O—C₁-C₄-alkylene-COOR_{A}⁵, C₀-C₄-alkylene-SR_{A}⁵, C₀-C₄-alkylene-C₃-C₇-cycloalkyl, C₀-C₄-alkylene-OCOR_{A}⁵, C₀-C₄-alkylene-SO₂NR_{A}⁶R_{A}⁷, C₀-C₄-alkylene-CONR_{A}⁶R_{A}⁷, C₁-C₄-alkylene-OCONR_{A}⁶R_{A}⁷, C₁-C₄-alkylene-SOR_{A}⁵, C₁-C₄-alkylene-SO₂R_{A}⁵, NHCOO—C₀-C₄-alkylene-aryl, NHCOOH, NH₂, NH(C₁-C₄-alkyl), N(C₁-C₄-alkyl)₂,
   where two of the radicals R_{A}¹, R_{A}², R_{A}³ and R_{A}⁴ in adjacent position (“ortho”) to one another may also form an optionally substituted, fused saturated, unsaturated and/or aromatic 3- to 10-membered carbocycle or a cyclic acetal —O—CH₂—CH₂—O— or —O—CH₂—O—,
   and in which
   R_{A}⁵ is independently of its respective occurrence hydrogen, a branched or unbranched radical C₁-C₆-alkyl, or a branched or unbranched, optionally substituted radical C₂-C₆-alkenyl-, C₂-C₆-alkynyl, C₃-C₇-cycloalkyl-, C₁-C₄-alkylene-C₃-C₇-cycloalkyl- or C₁-C₄-alkylene-(C₆-C₁₀)-aryl,
   R_{A}⁶ and R_{A}⁷ are independently of one another and independently of their respective occurrence hydrogen, a branched or unbranched, optionally substituted radical C₁-C₆-alkyl-, C₂-C₆-alkenyl-, C₂-C₆-alkynyl, C₁-C₅-alkylene-C₁-C₄-alkoxy-, C₃-C₇-cycloalkyl-, C₁-C₄-alkylene-C₃-C₇-cycloalkyl, C₁-C₄-alkylene-aryl, or a radical —SO₂R_{A}⁵, —CO₂R_{A}⁵, —CO—NR_{A}⁵ R_{A}⁵, or —COR_{A}⁵, and
B is an aromatic or partly aromatic C₆-C₁₀-mono or fused bicycle which may be substituted with a maximum of four radicals selected from the group consisting of R_{B}¹, R_{B}², R_{B}³, and R_{B}⁴, where R_{B}¹, R_{B}², R_{B}³ and R_{B}⁴ are selected independently of one another and independently of their respective occurrence from the group consisting of hydrogen, chlorine, bromine, iodine, fluorine, CN, OR_{B}⁵, COR_{B}⁵, COOR_{B}⁵, SR_{B}⁵, C₃-C₇-cycloalkyl, OCOR_{A}⁵, SO₂NR_{A}⁶R_{A}⁷, CONR_{A}⁶R_{A}⁷, (C₆-C₁₀)-aryl, (C₃-C₁₀)-hetaryl, NR_{B}⁶R_{B}⁷, C₃-C₇-heterocycloalkyl, C₃-C₇-heterocycloalkenyl, OCOR_{B}⁵, SO₂NR_{B}⁶R_{B}⁷, CONR_{B}⁶R_{B}⁷, C₀-C₄-alkylene-CN, C₁-C₆-haloalkyl, C₁-C₆-haloalkoxy, NO₂, C₀-C₄-alkylene-OR_{B}⁵, O—C₀-C₄—alkylene-(C₆-C₁₀)-aryl, O—C₀-C₄-alkylene-(C₂-C₁₀)-hetaryl, C₀-C₄-alkylene-(C₆-C₁₀)-aryl, C₀-C₄-alkylene-(C₂-C₁₀)-hetaryl, C₁-C₆-alkyl, C₂-C₆-alkenyl, C₂-C₆-alkynyl, C₂-C₆-alkenyloxy, C₂-C₆-alkynyloxy, C₁-C₄-alkylthio, C₀-C₄-alkylene-NR_{B}⁶R₆⁷, C₀-C₄-alkylene-COR_{B}⁵, SO₂R_{B}⁵, C₀-C₄-alkylene-COOR_{B}⁵, O—C₁-C₄-alkylene-COOR_{B}⁵, C₀-C₄-alkylene-SR_{B}⁵, C₀-C₄-alkylene-C₃-C₇-cycloalkyl, C₀-C₄-alkylene-C₃-C₇-heterocycloalkyl, C₀-C₄-alkylene-C₃-C₇-heterocycloalkenyl, C₀-C₄-alkylene-OCOR_{B}⁵, C₀-C₄-alkylene-SO₂NR_{B}⁶R_{B}⁷, C₀-C₄-alkylene-CONR_{B}⁶R_{B}⁷, C₁-C₄-alkylene-OCONR_{B}⁶R₆⁷, C₁-C₄-alkylene-SOR₆⁵, C₁-C₄- alkylene-SO₂R_{B}⁵, NHCOO—C₀-C₄-alkylene-(C₆-C₁₀)-aryl, NHCOO—(C₆-C₁₀)-aryl, NH₂, NH(C₁-C₄-alkyl), N(C₁-C₄-alkyl)₂, morpholin-4-yl, pyrrolidin-1-yl, piperidin-1-yl, 4-piperazin-1-yl, 4-(C₁-C₄-alkyl)-piperazin-1-yl;
   where two of the radicals R_{B}¹, R_{B}², R_{B}³, or R_{B}⁴ in adjacent (“ortho”) position to one another may also form a fused, unsaturated or aromatic 3- to 10-membered carbocycle which is optionally substituted one or more times identically or differently by the radicals C₁-C₆-alkyl-, OCH₃ or halogen,
   in which
   R_{B}⁵ is independently of its respective occurrence hydrogen, a branched or unbranched, optionally substituted C₁-C₆-alkyl, C₂-C₆-alkenyl, C₂-C₆-alkynyl, C₁-C₅-alkylene-C₁-C₄-alkoxy, mono- or bis-(C₁-C₆)-alkylamino-(C₁-C₄)-alkylene or (C₁-C₆)-acylamino-(C₁-C₄)-alkylene radical or an optionally substituted (C₆-C₁₀)-aryl-, C₃-C₇-heterocycloalkyl-, C₃-C₇-heterocycloalkenyl-, (C₂-C₁₀)-hetaryl, C₃-C₇-cycloalkyl-, C₁-C₄-alkylene-C₃-C₇-cycloalkyl-, C₁-C₄-alkylene-(C₆-C₁₀)-aryl, C₁-C₄-alkylene-C₃-C₇-heterocycloalkyl-, C₁-C₄-alkylene-C₃-C₇-heterocycloalkenyl- or C₁-C₄-alkylene-(C₂-C₁₀)-hetaryl,
   R_{B}⁶ and R_{B}⁷ are independently of one another and independently of their respective occurrence hydrogen, a branched or unbranched, optionally substituted C₁-C₆-alkyl, C₂-C₆-alkenyl, C₂-C₆-alkynyl, C₁-C₅-alkylene-C₁-C₄-alkoxy, mono- or bis-(C₁-C₆)-alkylamino-(C₁-C₄)-alkylene or (C₁-C₆)-acylamino-(C₁-C₄)-alkylene radical or an optionally substituted (C₆-C₁₀)-aryl, C₃-C₇-heterocycloalkyl, C₃-C₇-heterocycloalkenyl, (C₂-C₁₀)-hetaryl, C₃-C₇-cycloalkyl, C₁-C₄-alkylene-C₃-C₇-cycloalkyl, C₁-C₄-alkylene-(C₆-C₁₀)-aryl, C₁-C₄-alkylene-C₃-C₇-heterocyloalkyl, C₁-C₄-alkylene-C₃-C₇-heterocyloalkenyl or C₁-C₄-alkylene-(C₂-C₁₀)-hetaryl radical, or a radical —SO₂R_{B}⁵, —CO₂R_{B}⁵, —CO—NR_{B}⁵R_{B}⁵, or COR_{B}⁵;
   or R_{B}⁵ and R_{B}⁷ are independently of their respective occurrence together a 3- to 7-membered, optionally substituted, or preferably C₁-C₆-alkyl-, OCH₃—, halogen-substituted, saturated, unsaturated or aromatic heterocycle which, in addition to the ring nitrogen atom, may comprise up to three further different or identical heteroatoms selected from the group consisting of O, N and S, and optionally two radicals R^{x} and R^{x} substituted on this heterocycle together are a fused, saturated, unsaturated or aromatic carbocycle or heterocycle which may comprise up to three different or identical heteroatoms selected from the group consisting of O, N and S, and the ring may optionally be substituted or a further, optionally substituted ring may be fused to this ring,
X is one of the radicals hydrogen, Br, F, Cl, I, C₁-C₄-alkylene-CN, CN, C₁-C₆-haloalkyl, C₁-C₆-haloalkoxy, NO₂, C₁-C₄-alkylene-OR_{X}¹, OR_{X}¹, O—C₁-C₄-alkylene-(C₆-C₁₀)-aryl, O—(C₆-C₁₀)-aryl, O—C₁-C₄-alkylenehetaryl, e.g. O—C₁-C₄-alkylene-(C₃-C₁₀)-hetaryl, O-hetaryl, e.g. O—(C₃-C₁₀)-hetaryl, C₁-C₄-alkylene-(C₆-C₁₀)-aryl, (C₆-C₁₀)-aryl, C₁-C₄-alkylenehetaryl, e.g. C₁-C₄-alkylene-(C₃-C₁₀)-hetaryl, (C₂-C₁₀)-hetaryl or (C₃-C₁₀)-hetaryl, C₁-C₆-alkyl, C₂-C₆-alkenyl, C₂-C₆-alkynyl, C₂-C₆-alkenyloxy, C₂-C₆-alkynyloxy, C₁-C₄-alkylthio, C₁-C₄-alkylene-NR_{X}²R_{X}³, NR_{X}²R_{X}³, C₁-C₄-alkylene-COR_{X}¹, COR_{X}¹, SO₂R_{X}¹, C₁-C₄alkylene-COOR_{X}¹, COOR_{X}¹, O—C₁-C₄-alkylene-COOR_{X}¹, C₁-C₄-alkylene-SR_{X}¹, SR_{X}¹, C₁-C₄-alkylene-C₃-C₇-cycloalkyl, C₃-C₇-cycloalkyl, C₁-C₄-alkylene-C₃-C₇-heterocycloalkyl, C₃-C₇-heterocycloalkyl, C₁-C₄-alkylene-C₃-C₇-heterocycloalkenyl, C₃-C₇-heterocycloalkenyl, C₁-C₄-alkylene-OCOR_{X}¹, OCOR_{X}¹, C₁-C₄-alkylene-SO₂NR_{X}²R_{X}³, SO₂NR_{X}²R_{X}³, C₁-C₄-alkylene-CONR_{X}²R_{X}³, CONR_{X}²R_{X}³, C₁-C₄-alkylene-OCONR_{X}²R_{X}³, C₁-C₄-alkylene-SOR_{X}¹, C₁-C₄-alkylene-SO₂R_{X}¹, NHCOO—C₀-C₄-alkylene-(C₆-C₁₀)-aryl or NHCOO—(C₆-C₁₀)-aryl,
   in which
   R_{X}¹ is independently of its respective occurrence hydrogen, a branched or unbranched, optionally substituted radical C₁-C₆-alkyl-, C₂-C₆-alkenyl-, C₂-C₆-alkynyl, C₁-C₅-alkylene-C₁-C₄-alkoxy-, mono- or bis-(C₁-C₆)-alkylamino-(C₁-C₄)-alkylene- or (C₁-C₆)-acylamino-(C₁-C₄)-alkylene or an optionally substituted (C₆-C₁₀)-aryl-, C₃-C₇-heterocycloalkyl-, C₃-C₇-heterocycloalkenyl-, (C₂-C₁₀)-hetaryl or (C₃-C₁₀)-hetaryl, C₃-C₇-cycloalkyl-, C₁-C₄-alkylene-C₃-C₇-cycloalkyl-, C₁-C₄-alkylene-(C₆-C₁₀)-aryl, C₁-C₄-alkylene-C₃-C₇-heterocyloalkyl, C₁-C₄-alkylene-C₃-C₇-heterocyloalkenyl or C₁-C₄-alkylene-(C₂-C₁₀)-hetaryl or C₁-C₄-alkylene-(C₃-C₁₀)-hetaryl,
   R_{X}² and R_{X}³ are independently of one another and independently of their respective occurrence hydrogen, a branched or unbranched, optionally substituted radical C₁-C₆-alkyl-, C₂-C₆-alkenyl-, C₂-C₆-alkynyl, C₁-C₅-alkylene-C₁-C₄-alkoxy-, mono- or bis-(C₁-C₆)-alkylamino-(C₁-C₄)-alkylene- or (C₁-C₆)-acylamino-(C₁-C₄)-alkylene or an optionally substituted radical (C₆-C₁₀)-aryl-, C₃-C₇-heterocycloalkyl-, C₃-C₇-heterocycloalkenyl-, (C₂-C₁₀)-hetaryl or (C₃-C₁₀)-hetaryl, C₃-C₇-cycloalkyl-, C₁-C₄-alkylene-C₃-C₇-cycloalkyl-, C₁-C₄-alkylene-(C₆-C₁₀)-aryl, C₁-C₄-alkylene-C₃-C₇-heterocyloalkyl-, C₁-C₄-alkylene-C₃-C₇-heterocyloalkenyl- or C₁-C₄-alkylene-(C₂-C₁₀)-hetaryl or C₁-C₄-alkylene-(C₃-C₁₀)-hetaryl, or a radical —SO₂R_{X}¹, —CO₂R_{X}¹, —CO—NR_{X}¹R_{X}¹, or COR_{X}¹, or R_{X}² and R_{X}³ together are a 3, 4, 5, 6 or 7 membered, optionally substituted, optionally preferably substituted by C₁-C₆-alkyl-, OCH₃, and/or halogen, saturated, unsaturated or aromatic (C₂-C₁₀)— or C₃-C₁₀-heterocycle which, in addition to the ring nitrogen atom, may comprise one, two or three further different or identical heteroatoms selected from the group consisting of O, N, and S, and optionally two radicals Rₓ⁴ and R_{X}⁵ substituted on this heterocycle are together a mono- or fused bi- or tricycle having a total of 3 to 21 ring atoms, which may in each case be saturated, unsaturated or aromatic and optionally be substituted by up to six radicals selected from the group consisting of C₁-C₆-alkyl-, OCH₃ and halogen, where at least one ring may comprise a ring nitrogen atom, and additionally up to three further different or identical heteroatoms selected from the group consisting of O, N and S may be present in each ring independently of one another, e.g. two radicals R_{X}⁴ and R_{X}⁵ substituted on this heterocycle may together form a fused, saturated, unsaturated or aromatic C₆-C₁₀-carbocycle or C₂-C₁₀-heterocycle which has up to 3 different or identical heteroatoms selected from O, N and S, where the ring is optionally substituted, or a further, optionally substituted ring may be fused onto this ring;
Y is a radical

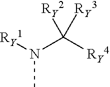

in which
   R_{Y}¹ is H, C₁-C₆-alkyl or C₃-C₇-cycloalkyl,
   R_{Y}² is H, C₁-C₆-alkyl or C₃-C₇-cycloalkyl,
in which R_{Y}¹ and R_{Y}² may also together form a 4-, 5-, 6- or 7-membered, saturated or unsaturated ring which may include a heteroatom selected from the group consisting of O, S and NR_{Y}⁵ as ring member, where R_{Y}⁵ may be independently of its respective occurrence hydrogen, C₁-C₄-alkyl or C₃-C₇-cycloalkyl, and where the ring may include one or two substituents R_{Y}⁶ and R_{Y}⁷ which are selected independently of one another and independently of their respective occurrence from the group consisting of the radicals C₁-C₆-alkyl, CN, OR_{Y}⁸, NR_{Y}⁹R_{Y}¹⁰, CONR_{Y}⁹R_{Y}¹⁰ and halogen;
or
R_{Y}⁶ and R_{Y}⁷ may also form independently of their respective occurrence together with the C atoms to which they are bonded a fused phenyl ring or a fused 5- or 6-membered aromatic heterocycle which includes 1, 2, 3 or 4 heteroatoms which are selected from the group consisting of N, O and S, where the fused phenyl ring and/or the fused aromatic heterocycle may include independently of one another one, two or three substituents R_{Y}¹⁴ which are selected independently of one another and independently of their respective occurrence from the group consisting of the radicals C₁-C₆-alkyl, CN, OR_{Y}⁸, NR_{Y}⁹R_{Y}¹⁰, NO₂, SR_{Y}¹¹, SO₂R_{Y}¹¹, SO₂NR_{Y}⁹R_{Y}¹⁰, CONR_{Y}⁹R_{Y}¹⁰, COOR_{Y}¹², COR_{Y}¹³, C₁-C₄-haloalkoxy, C₂-C₆-alkenyl, C₂-C₆-alkynyl, C₂-C₆-alkenyloxy, C₂-C₆-alkynyloxy, C₃-C₇-cycloalkyl, C₃-C₇-cycloalkyloxy and halogen;
in which
R_{Y}⁸, R_{Y}⁹, R_{Y}¹⁰, R_{Y}¹¹, R_{Y}¹² and R_{Y}¹³ are selected independently of one another and independently of their respective occurrence from the group consisting of H, optionally substituted C₁-C₆-alkyl, optionally substituted C₃-C₇-cycloalkyl and optionally substituted phenyl,
where R_{Y}⁸ may also independently of its respective occurrence be a radical —(CH₂)ₙ, —COR_{Y}¹⁵ or —CO—(CH₂)ₙ—CONR_{Y}¹⁶R_{Y}¹⁷,
in which
R_{Y}¹⁵ is independently of its respective occurrence H, OH, C₁-C₆-alkyl, C₁-C₅-alkoxy, C₃-C₇-cycloalkyl, CH₂CH₂COOH, NR_{Y}¹⁸R_{Y}¹⁹, preferably H, CH₃, C₂H₅, isopropyl, cyclohexyl, —CH₂CH₂COOH, NH₂, N(CH₃)₂;
R_{Y}¹⁶ and R_{Y}¹⁷ are independently of one another and their respective occurrence, selected from the group consisting of H, C₁-C₆-alkyl and C₃-C₆-cycloalkyl; or R_{Y}¹⁶ and R_{Y}¹⁷ independently of their respective occurrence may together form a ring selected from the group consisting of azetidin-1-yl, pyrrolidin-1-yl, piperidin-1-yl, piperazin-1-yl, morpholin-4-yl and thiomorpholin-4-yl;
R_{Y}¹⁸ is independently of its respective occurrence H, C₁-C₆-alkyl, or C₃-C₆-cycloalkyl;
R_{Y}¹⁹ is independently of its respective occurrence H, C₁-C₆-alkyl, C₃-C₆-cycloalkyl, —C(CH₃)₂CH₂OH, —C(CH₃)(CH₂OH)₂, or —C(CH₂OH)₃; or R_{Y}¹⁸ and R_{Y}¹⁹ independently of their respective occurrence may together form a ring selected from the group consisting of azetidin-1-yl, pyrrolidin-1-yl, piperidin-1-yl, piperazin-1-yl, morpholin-4-yl and thiomorpholin-4-yl;
R_{Y}¹⁰ may also independently of its respective occurrence be a group COR_{Y}²⁰ in which R_{Y}²⁰ is independently of its respective occurrence hydrogen, optionally substituted C₁-C₄-alkyl or optionally substituted phenyl, or where R_{Y}⁹ may also form with R_{Y}¹⁹ irrespective of their respective occurrence together a 5- or 6-membered, saturated or unsaturated carbocycle which may include a heteroatom selected from the group consisting of O, S, and NR_{Y}¹⁴, as ring member, where R_{Y}¹⁴ is hydrogen or C₁-C₄-alkyl,
n is independently of its respective occurrence the integer 1 or 2;
R_{Y}³ is independently of its respective occurrence H, C₁-C₆-alkyl or C₃-C₆-cycloalkyl, preferably H;
R_{Y}⁴ is independently of its respective occurrence H, CONR_{Y}²¹R_{Y}²², CR_{Y}²³R_{Y}²⁴NR_{Y}²¹R_{Y}²², COOH or COO—C₁-C₄-alkyl;
R_{Y}²¹, R_{Y}²², RY²³ and RY²⁴ are independently of one another and independently of their respective occurrence H, C₁-C₆-alkyl or C₃-C₆-cycloalkyl;
or R_{Y}²¹ and R_{Y}²² may also independently of their respective occurrence together form a 4-, 5- or 6-membered, saturated or unsaturated carbocycle which may include a heteroatom selected from the group consisting of O, S, and NR_{Y}²⁵, as ring member, where R_{Y}²⁵ is independently of its respective occurrence hydrogen or C₁-C₄-alkyl;
the tautomeric, enantiomeric and diastereomeric forms thereof, and the prodrugs thereof, and the physiologically tolerated salts of said compounds.

Any of these aforementioned definitions of a variable can be combined with any of the aforementioned definitions of the remaining variables.

Oxindole derivates which are preferred according to the invention are the compounds I in which the variables A, B, X and Y have independently of one another one of the meaning indicated in any of dependent claims 2 to 8.

The variables A and B independently of one another and especially in combination preferably have one of the following meanings:
A is C₆-C₁₀-aryl which may be substituted by one, two, three or four radicals selected from the group consisting of R_{A}¹, R_{A}², R_{A}³ and R_{A}⁴, in which R_{A}¹, R_{A}², R_{A}³ and R_{A}⁴ are selected independently of one another and independently of their respective occurrence from the group consisting of hydrogen, chlorine, bromine, iodine, fluorine, CN, OR_{A}⁵, COR_{A}⁵, COOR_{A}⁵, SR_{A}⁵, C₃-C₇-cycloalkyl, OCOR_{A}⁵, SO₂NR_{A}⁶R_{A}⁷, CONR_{A}⁶R_{A}⁷, C₀-C₄-alkylene-CN, C₁-C₆-haloalkyl, C₁-C₆-haloalkoxy, NO₂, C₀-C₄-alkylene-OR_{A}⁵, C₁-C₆-alkyl, C₂-C₆-alkenyl, C₂-C₆-alkynyl, C₂-C₆-alkenyloxy, C₂-C₆-alkynyloxy, C₁-C₄-alkylthio, C₀-C₄-alkylene-COR_{A}⁵, SO₂R_{A}⁵, C₀-C₄-alkylene-COOR_{A}⁵, O—C₁-C₄-alkylene-COOR_{A}⁵, C₀-C₄-alkylene-SR_{A}⁵, C₀-C₄-alkylene-C₃-C₇-cycloalkyl, C₀-C₄-alkylene-OCOR_{A}⁵, C₀-C₄-alkylene-SO₂NR_{A}⁶R_{A}⁷, C₀-C₄-alkylene-CONR_{A}⁶R_{A}⁷, C₁-C₄-alkylene-OCONR_{A}⁶R_{A}⁷, C₁-C₄-alkylene-SOR_{A}⁵, C₁-C₄-alkylene-SO₂R_{A}⁵, NHCOO—C₀-C₄-alkylene-aryl and NHCOOH,
   where two of the radicals R_{A}¹, R_{A}², R_{A}³ and R_{A}⁴ in adjacent position (“ortho”) to one another may also form an optionally substituted, fused saturated, unsaturated or aromatic 3- to 10-membered carbocycle or a cyclic acetal —O—CH₂—CH₂—O— or —O—CH₂—O—,
   and in which
   R_{A}⁵ is independently of its respective occurrence hydrogen, a branched or unbranched radical C₁-C₆-alkyl, or a branched or unbranched, optionally substituted radical C₂-C₆-alkenyl-, C₂-C₆-alkynyl, C₃-C₇-cycloalkyl-, C₁-C₄-alkylene-C₃-C₇-cycloalkyl- or C₁-C₄-alkylene-(C₆-C₁₀)-aryl,
   R_{A}⁶ and R_{A}⁷ are independently of one another and independently of their respective occurrence hydrogen, a branched or unbranched, optionally substituted radical C₁-C₆-alkyl-, C₂-C₆-alkenyl-, C₂-C₆-alkynyl, C₁-C₅-alkylene-C₁-C₄-alkoxy-, C₃-C₇-cycloalkyl-, C₁-C₄-alkylene-C₃-C₇-cycloalkyl, C₁-C₄-alkylene-aryl, or a radical —SO₂R_{A}⁵, —CO₂R_{A}⁵, —CO—NR_{A}⁵ R_{A}⁵, or —COR_{A}⁵, and
B is an aromatic or partly aromatic C₆-C₁₀-mono or fused bicycle which may be substituted by a maximum of four radicals selected from the group consisting of R_{B}¹, R_{B}², R_{B}³, and R_{B}⁴, where R_{B}¹, R_{B}², R_{B}³ and R_{B}⁴ are selected independently of one another and independently of their respective occurrence from the group consisting of hydrogen, chlorine, bromine, iodine, fluorine, CN, OR_{B}⁵, COR_{B}⁵, COOR_{B}⁵, SR_{B}⁵, C₃-C₇-cycloalkyl, OCOR_{A}⁵, SO₂NR_{A}⁶R_{A}⁷, CONR_{A}⁶R_{A}⁷, (C₆-C₁₀-aryl, (C₃-C₁₀)-hetaryl, NR_{B}⁶R_{B}⁷, C₃-C₇-heterocycloalkyl, C₃-C₇-heterocyclo-alkenyl, OCOR_{B}⁵, SO₂NR_{B}⁶R_{B}⁷, CONR_{B}⁶R_{B}⁷, C₀-C₄-alkylene-CN, C₁-C₆-haloalkyl, C₁-C₆-haloalkoxy, NO₂, C₀-C₄-alkylene-OR_{B}⁵, O—C₀-C₄-alkylene-(C₆-C₁₀)-aryl, O—C₀-C₄-alkylene-(C₂-C₁₀)-hetaryl, C₀-C₄-alkylene-(C₆-C₁₀)-aryl, C₀-C₄-alkylene-(C₂-C₁₀)-hetaryl, C₁-C₆-alkyl, C₂-C₆-alkenyl, C₂-C₆-alkynyl, C₂-C₆-alkenyloxy, C₂-C₆-alkynyloxy, C₁-C₄-alkylthio, C₀-C₄-alkylene-NR_{B}⁶R_{B}⁷, C₀-C₄-alkylene-COR_{B}⁵, SO₂R_{B}⁵, C₀-C₄-alkylene-COOR_{B}⁵, O—C₁-C₄-alkylene-COOR_{B}⁵, C₀-C₄-alkylene-SR_{B}⁵, C₀-C₄-alkylene-C₃-C₇-cycloalkyl, C₀-C₄-alkylene-C₃-C₇-heterocycloalkyl, C₀-C₄-alkylene-C₃-C₇-heterocycloalkenyl, C₀-C₄-alkylene-OCOR_{B}⁵, C₀-C₄-alkylene-SO₂NR_{B}⁶R_{B}⁷, C₀-C₄-alkylene-CONR_{B}⁶R_{B}⁷, C₁-C₄-alkylene-OCONR_{B}⁶R_{B}⁷, C₁-C₄-alkylene-SOR_{B}⁵, C₁-C₄-alkylene-SO₂R_{B}⁵, NHCOO—C₀-C₄-alkylene-(C₆-C₁₀)-aryl and NHCOO—(C₆-C₁₀)-aryl,
   where two of the radicals R_{B}¹, R_{B}², R_{B}³, or R_{B}⁴ in adjacent (“ortho”) position to one another may also form a fused, unsaturated or aromatic 3- to 10-membered carbocycle which is optionally substituted one or more times identically or differently by the radicals C₁-C₆-alkyl-, OCH₃ or halogen,
   in which
   R_{B}⁵ is independently of its respective occurrence hydrogen, a branched or unbranched, optionally substituted C₁-C₆-alkyl, C₂-C₆-alkenyl, C₂-C₆-alkynyl, C₁-C₅-alkylene-C₁-C₄-alkoxy, mono- or bis-(C₁-C₆)-alkylamino-(C₁-C₄)-alkylene or (C₁-C₆)-acylamino-(C₁-C₄)-alkylene radical or an optionally substituted (C₆-C₁₀)-aryl-, C₃-C₇-heterocycloalkyl-, C₃-C₇-heterocycloalkenyl-, (C₂-C₁₀₋hetaryl, C₃-C₇-cycloalkyl-, C₁-C₄-alkylene-C₃-C₇-cycloalkyl-, C₁-C₄-alkylene-(C₆-C₁₀)-aryl, C₁-C₄-alkylene-C₃-C₇-heterocyloalkyl-, C₁-C₄-alkylene-C₃-C₇-heterocyloalkenyl- or C₁-C₄-alkylene-(C₂-C₁₀)-hetaryl,
   R_{B}⁶ and R_{B}⁷ are independently of one another and independently of their respective occurrence hydrogen, a branched or unbranched, optionally substituted C₁-C₆-alkyl-, C₂-C₆-alkenyl-, C₂-C₆-alkynyl, C₁-C₅-alkylene-C₁-C₄-alkoxy-, mono- or bis(C₁-C₆)-alkylamino-(C₁-C₄)-alkylene or (C₁-C₆)-acylamino-(C₁-C₄)-alkylene radical or an optionally substituted (C₆-C₁₀)-aryl-, C₃-C₇-heterocycloalkyl-, C₃-C₇-heterocycloalkenyl-, (C₂-C₁₀)-hetaryl, C₃-C₇-cycloalkyl-, C₁-C₄-alkylene-C₃-C₇-cycloalkyl-, C₁-C₄-alkylene-(C₆-C₁₀)-aryl, C₁-C₄-alkylene-C₃-C₇-heterocycloalkyl-, C₁-C₄-alkylene-C₃-C₇-heterocycloalkenyl- or C₁-C₄-alkylene-(C₂-C₁₀)-hetaryl, or a radical —SO₂R_{B}⁵, —CO₂R_{B}⁵, —CO—NR_{B}⁵R_{B}⁵, or COR_{B}⁵;
   or R_{B}⁶ and R_{B}⁷ are independently of their respective occurrence together a 3 to 7 membered, optionally substituted, or preferably C₁-C₆-alkyl-, OMe-, halogen-substituted, saturated, unsaturated or aromatic heterocycle which, in addition to the ring nitrogen atom, may comprise up to three further different or identical heteroatoms selected from the group consisting of O, N and S, and optionally two radicals R^{x} and R^{x} substituted on this heterocycle are together a fused, saturated, unsaturated or aromatic carbocycle or heterocycle which may comprise up to three different or identical heteroatoms selected from the group consisting of O, N and S, and the ring may optionally be substituted, or a further, optionally substituted ring may be fused to this ring,

The variables A and B especially have independently of one another and especially in combination one of the following meanings:
A is C₆-C₁₀-aryl which may be substituted by one, two, three or four radicals selected from the group consisting of R_{A}¹, R_{A}², R_{A}³ and/or R_{A}⁴, in which R_{A}¹, R_{A}², R_{A}³ and R_{A}⁴ are selected independently of one another and independently of their respective occurrence from the group consisting of hydrogen, chlorine, bromine, iodine, fluorine, CN, OR_{A}⁵, SR_{A}⁵, C₃-C₇-cycloalkyl, NO₂, C₁-C₆-alkyl, C₂-C₆-alkenyl, C₂-C₆-alkynyl, SO₂R_{A}⁵, C₁-C₄-alkylene-C₃-C₇-cycloalkyl, CF₃, OCHF₂, NH₂, NH(C₁-C₄-alkyl), N(C₁-C₄-alkyl)₂,
   where two of the radicals R_{A}¹, R_{A}², R_{A}³ and R_{A}⁴ in adjacent position (“ortho”) to one another may also form an optionally substituted, fused saturated, unsaturated and/or aromatic 3- to 10-membered carbocycle or a cyclic acetal —O—CH₂—CH₂—O— or —O—CH₂—O—,
   and in which
   R_{A}⁵ is independently of its respective occurrence hydrogen, a branched or unbranched radical C₁-C₆-alkyl, or a branched or unbranched, optionally substituted radical C₂-C₆-alkenyl-, C₂-C₆-alkynyl, C₃-C₇-cycloalkyl-, C₁-C₄-alkylene-C₃-C₇cycloalkyl- or C₁-C₄-alkylene-(C₆-C₁₀)-aryl,
B is an aromatic or partly aromatic C₆-C₁₀-mono- or fused -bicycle which may be substituted by one, two or three radicals selected from the group consisting of R_{B}¹, R_{B}² and/or R_{B}³, where R_{B}¹, R_{B}² and R_{B}³ are selected independently of one another and independently of their respective occurrence from the group consisting of hydrogen, chlorine, bromine, iodine, fluorine, CN, CF₃, OCF₃, NO₂, OH, O—C₁-C₄-alkyl, O-phenyl, O—C₁-C₄-alkylene-phenyl, phenyl, C₁-C₆-alkyl, C₂-C₆-alkenyl, C₂-C₆-alkynyl, NH₂, NH(C₁-C₄-alkyl), N(C₁-C₄-alkyl)₂, morpholin-4-yl, pyrrolidin-1-yl, piperidin-1-yl, 4-piperazin-1-yl, 4-(C₁-C₄-alkyl)piperazin-1-yl;

In a preferred embodiment, at least one compound of the aforementioned general formula (I) are provided, characterized in that the variables A, B, X and Y have independently of one another, but preferably in combination, the following meanings.
A is a phenyl ring which may be substituted by one, two, three or four radicals selected from the group consisting of R_{A}¹, R_{A}², R_{A}³ and R_{A}⁴, where R_{A}¹, R_{A}², R_{A}³ and R_{A}⁴ have the meanings stated in claim 1;
B is a phenyl ring which may be substituted by one, two, three or four radicals selected from the group consisting of R_{B}¹, R_{B}², R_{B}³ and R_{B}⁴, R_{B}¹, R_{B}², R_{B}³ and R_{B}⁴ have the meaning stated in claim 1;
X is hydrogen, F, Cl, CF₃, OCF₃, O—C₁-C₄-alkyl, OH, C₁-C₆-alkyl or C₃-C₆-cycloalkyl; and
Y has the meaning stated in claim 1,
the tautomeric, enantiomeric and diastereomeric forms thereof, and the prodrugs thereof, and the physiologically tolerated salts of said compounds.

Each of these aforementioned preferred definitions of a variable can be combined with any of the aforementioned preferred definitions of the remaining variables.

In a further preferred embodiment, at least one compound of the aforementioned general formula (I) are provided, characterized in that
A is a phenyl ring which may be substituted by one or two radicals selected from the group consisting of R_{A}¹ and R_{A}² which are selected independently of one another from the group consisting of hydrogen, chlorine, fluorine, O—C₁-C₄-alkyl, OH, (CH₂)₁₋₂—O—(CH₂)₁₋₂—CH₃, O—(CH₂)₁₋₂—CH₃, OCH₃, (CH₂)₁₋₂—OCH₃, C₁-C₆-alkyl, C₃-C₇-cycloalkyl, O—C₃-C₆-cycloalkyl, CN, CF₃, OCF₃,
where R_{A}¹ and R_{A}² may also in adjacent position (“ortho”) together form a cyclic acetal —O—CH₂—O—;
B is a phenyl ring which may be substituted by one, two, three or four radicals selected from the group consisting of R_{B}¹, R_{B}², R_{B}³ and R_{B}⁴, where R_{B}¹, R_{B}², R_{B}³ and R_{B}⁴ selected independently of one another and independently of their respective occurrence from the group consisting of hydrogen, fluorine, chlorine, CF₃, OCF₃, OCHF₂, CN, O—C₁-C₄-alkyl, OH, C₁-C₆-alkyl and C₃-C₇-cycloalkyl,
or where two adjacent radicals R_{B}¹, R_{B}², R_{B}³ or R_{B}⁴ in adjacent position (“ortho”) may together form an optionally substituted, fused, saturated and/or aromatic 3- to 10-membered carbocycle;
X is hydrogen;
Y is a radical

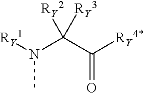

in which
   R_{Y}¹ is H, C₁-C₆-alkyl or C₃-C₆-cycloalkyl,
   R_{Y}² is H, C₁-C₆-alkyl or C₃-C₆-cycloalkyl,
   R_{Y}³ is H, C₁-C₆-alkyl or C₃-C₆-cycloalkyl,
   R_{Y}^{4*} is NR_{Y}¹³R_{Y}¹⁴ or O—C₁-C₄-alkyl
   in which
   R_{Y}¹³ is H, C₁-C₆-alkyl or C₃-C₆-cycloalkyl,
   R_{Y}¹⁴ is H, C₁-C₆-alkyl or C₃-C₆-cycloalkyl,
   where R_{Y}¹³ and R_{Y}¹⁴ may also together form a 4-, 5- or 6-membered, saturated or unsaturated ring which may include a heteroatom selected from the group consisting of O, S and NR_{Y}¹⁷ as ring member, where R_{Y}¹⁷ is hydrogen, C₁-C₄-alkyl or C₃-C₇-cycloalkyl-,
or Y is a radical

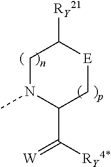

in which
n is 0, 1 or 2,
p is 0, 1 or 2,
with the proviso that the total of n and p is 1 or 2,
E is O, S, NR_{Y}²³ or C(C₁-C₄-alkyl)R_{Y}²², CHR_{Y}²²
W is O or S,
R_{Y}^{4*} is NR_{Y}¹³R_{Y}¹⁴ or O—C₁-C₄-alkyl,
R_{Y}¹³ is H, C₁-C₆-alkyl or C₁-C₆-cycloalkyl,
R_{Y}¹⁴ is H, C₁-C₆-alkyl or C₁-C₆-cycloalkyl,
where R_{Y}¹³ and R_{Y}¹⁴ may also together form a 4-, 5- or 6-membered, saturated or unsaturated ring which may include a heteroatom selected from the group consisting of O, S and NR_{Y}¹⁷ as ring member, where R_{Y}¹⁷ is hydrogen, C₁-C₄-alkyl or C₃-C₆-cycloalkyl.
R_{Y}²¹ is selected from the group consisting of the radicals optionally substituted C₁-C₆-alkyl, optionally substituted C₃-C₆-cycloalkyl, CN, OR_{Y}⁸, NR_{Y}⁹R_{Y}¹⁰, CONR_{Y}⁹R_{Y}¹⁰ and halogen; in which

R_{Y}⁸, R_{Y}⁹, R_{Y}¹⁰, R_{Y}¹¹, R_{Y}¹², R_{Y}¹³, are independently of one another and independently of their respective occurrence H, optionally substituted C₁-C₅-alkyl, optionally substituted C₃-C₆-cycloalkyl or optionally substituted phenyl,
where
R_{Y}⁸ may also independently of its respective occurrence and independently of its respective occurrence be a radical —(CH₂)_{q}—COR_{Y}¹⁵ or —CO—(CH₂)_{q}—CONR_{Y}¹⁶R_{Y}¹⁷;
in which
R_{Y}¹⁵ is independently of its respective occurrence H, OH, C₁-C₆-alkyl, C₁-C₅-alkoxy, C₃-C₇-cycloalkyl, CH₂CH₂COOH, NR_{Y}¹⁸R_{Y}¹⁹, preferably H, CH₃, C₂H₅, iso-C₃H₇, cyclohexyl, —CH₂CH₂COOH, NH₂ or N(CH₃)₂;
R_{Y}¹⁶ and R_{Y}¹⁷ are independently of one another and independently of their respective occurrence H, C₁-C₆-alkyl or C₃-C₆-cycloalkyl, or R_{Y}¹⁶ and R_{Y}¹⁷ may independently of their respective occurrence together also be a ring selected from the group consisting of azetidin-1-yl, pyrrolidin-1-yl, piperidin-1-yl, piperazin-1-yl, morpholin-4-yl and thiomorpholin-4-yl;
R_{Y}¹⁸ is independently of its respective occurrence H, C₁-C₆-alkyl or C₃-C₆-cycloalkyl;
R_{Y}¹⁹ is independently of its respective occurrence H, C₁-C₆-alkyl or C₃-C₆-cycloalkyl, —C(CH₃)₂CH₂OH, —C(CH₃)(CH₂OH)₂, —C(CH₂OH)₃, or R_{Y}¹⁸ and R_{Y}¹⁹ may independently of their respective occurrence together form a ring selected from the group consisting of azetidin-1-yl, pyrrolidin-1-yl, piperidin-1-yl, piperazin-1-yl, morpholin-4-yl and thiomorpholin-4-yl;
R_{Y}¹⁰ may independently of its respective occurrence also be a group COR_{Y}²⁰, where R_{Y}²⁰ is hydrogen, optionally substituted C₁-C₄-alkyl or optionally substituted phenyl,
or R_{Y}⁹ and R_{Y}¹⁰ may also independently of their respective occurrence together form a 5- or 6-membered, saturated or unsaturated carbocycle which may include a heteroatom selected from the group consisting of O, S and NR_{Y}¹⁴ as ring member, where R_{Y}¹⁴ is hydrogen or C₁-C₄-alkyl,
where
   q is independently of its respective occurrence 1 or 2,
   R_{Y}²² is H or C₁-C₄-alkyl,
   Or
   R_{Y}²¹ and R_{Y}²² may also independently of their respective occurrence form together with the C atoms to which they are bonded a fused phenyl ring or a fused 5- or 6-membered aromatic heterocycle which includes 1, 2, 3 or 4 heteroatoms which are selected from the group of N, O and S, where the fused phenyl ring and the fused aromatic heterocycle may independently of one another have 1, 2 or 3 substituents which are selected independently of one another from the group consisting of optionally substituted C₁-C₆-alkyl, CN, OR_{Y}⁸, NR_{Y}⁹R_{Y}¹⁰, NO₂, SR_{Y}¹¹, SO₂R_{Y}¹¹, SO₂NR_{Y}⁹R_{Y}¹⁰, CONR_{Y}⁹R_{Y}¹⁰, COOR_{Y}¹², COR_{Y}¹³, C₁-C₄-haloalkoxy, C₂-C₆-alkenyl, C₂-C₆-alkynyl, C₂-C₆-alkenyloxy, C₂-C₆-alkynyloxy, C₃-C₆-cycloalkyl, C₃-C₆-cycloalkyloxy and halogen; in which
   R_{Y}²³ is independently of its respective occurrence H, C₁-C₆-alkyl, C₃-C₆-cycloalkyl or COCH₃;
the tautomeric, enantiomeric and diastereomeric forms thereof, and the prodrugs thereof, and the physiologically tolerated salts of said compounds, are provided.

Each of these aforementioned preferred definitions of a variable can be combined with any of the aforementioned preferred definitions of the remaining variables.

In a further preferred embodiment, at least one compound of the aforementioned general formula (I) are provided, characterized in that
A is the group

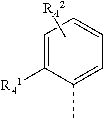

in which
R_{A}¹ and R_{A}² are selected independently of one another from the group consisting of hydrogen, chlorine, fluorine, O—C₁-C₄-alkyl, OH, (CH₂)₁₋₂—O—(CH₂)₁₋₂—CH₃, O—(CH₂)₁₋₂—CH₃, (CH₂)₁₋₂—OCH₃, OCH₃, C₁-C₆-alkyl, C₃-C₇-cycloalkyl, O—C₃-C₇-cycloalkyl, CN, CF₃ and OCF₃,
where R_{A}¹ and R_{A}² in adjacent position (“ortho”) may also together form a cyclic acetal —O—CH2—O—;
B is a phenyl ring which may be substituted by the radicals R_{B}¹ and R_{B}², where R_{B}¹ and R_{B}² are selected independently of one another from the group consisting of hydrogen, fluorine, chlorine, CN, CF₃, OCF₃, OCHF₂, OH, O—C₁-C₄-alkyl, C₁-C₆-alkyl and C₃-C₇-cycloalkyl,
X is hydrogen,
Y is a radical selected from the group consisting of

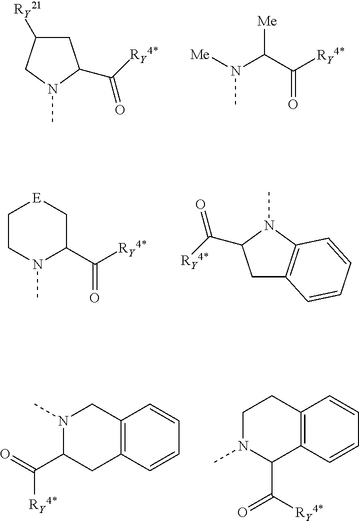

in which R_{Y}²¹ is selected from the group consisting of hydrogen, optionally substituted C₁-C₆-alkyl, optionally substituted C₃-C₇-cycloalkyl, CN, OR_{Y}⁸, NR_{Y}⁹R_{Y}¹⁰, CONR_{Y}⁹R_{Y}¹⁰ and halogen; in which
R_{Y}⁸, R_{Y}⁹ and R_{Y}¹⁰ are independently of one another and independently of their respective occurrence H, optionally substituted C₁-C₆-alkyl, optionally substituted C₃-C₇-cycloalkyl or optionally substituted phenyl,
where
R_{Y}⁸ may also be a radical —(CH₂)ₙ—COR_{Y}¹⁵ or —CO—(CH₂)ₙ—CONR_{Y}¹⁶R_{Y}¹⁷, in which
R_{Y}¹⁵ is independently of its respective occurrence H, OH, C₁-C₆-alkyl, C₁-C₆-alkoxy, C₃-C₇-cycloalkyl, CH₂CH₂COOH, NR_{Y}¹⁸R_{Y}¹⁹, preferably H, CH₃, C₂H₅, iso-C₃H₇, cyclohexyl, —CH₂CH₂COOH, NH₂, N(CH₃)₂;
R_{Y}¹⁶ and R_{Y}¹⁷ are independently of one another and independently of their respective occurrence H, C₁-C₆-alkyl or C₃-C₆-cycloalkyl,
or R_{Y}¹⁶ and R_{Y}¹⁷ may together form a ring selected from the group consisting of azetidin-1-yl, pyrrolidin-1-yl, piperidin-1-yl, piperazin-1-yl, morpholin-4-yl and thiomorpholin-4-yl;
R_{Y}¹⁸ is independently of its respective occurrence H, C₁-C₆-alkyl or C₃-C₆-cycloalkyl,
R_{Y}¹⁹ is independently of its respective occurrence H, C₁-C₆-alkyl, C₃-C₆-cycloalkyl, —C(CH₃)₂CH₂OH, —C(CH₃)(CH₂OH)₂, or —C(CH₂OH)₃, or R_{Y}¹⁸ and R_{Y}¹⁹ may independently of their respective occurrence together form a ring selected from the group consisting of azetidin-1-yl, pyrrolidin-1-yl, piperidin-1-yl, piperazin-1-yl, morpholin-4-yl and thiomorpholin-4-yl;
and in which
R_{Y}¹⁰ may also independently of its respective occurrence be a radical COR_{Y}²⁰ in which R_{Y}²⁰ is hydrogen, optionally substituted C₁-C₄-alkyl or optionally substituted phenyl,
where R_{Y}⁹ and R_{Y}¹⁰ may independently of their respective occurrence also together form a 5- or 6-membered, saturated or unsaturated carbocycle which may include a heteroatom selected from the group consisting of O, S and NR_{Y}¹⁴ as ring member, in which R_{Y}¹⁴ is hydrogen or C₁-C₄-alkyl,
n independently of its respective occurrence is the integer 1 or 2,
R_{Y}^{4*} is NHEt, NMe₂ or azetidinyl,
E is O or CH₂,
the tautomeric, enantiomeric and diastereomeric forms thereof, and the prodrugs thereof, and the physiologically tolerated salts of said compounds.

Each of these aforementioned preferred definitions of a variable can be combined with any of the aforementioned preferred definitions of the remaining variables.

In a further preferred embodiment, at least one compound of the aforementioned general formula (I) are provided, characterized in that
A is the group

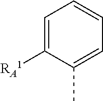

in which
R_{A}¹ is selected from the group consisting of chlorine, methyl, ethyl, OCH₃, OC₂H₅, OC₃H₇, O-i-C₃H₇, fluorine, CF₃, and OCF₃;
B is a phenyl ring which may be substituted by the radicals R_{B}¹ and R_{B}², where R_{B}¹ and R_{B}² are selected independently of one another from the group consisting of hydrogen, fluorine, chlorine, CN, CF₃, OCF₃, OCHF₂, OH, O—C₁-C₄-alkyl, C₃-C₇-cycloalkyl and C₁-C₆-alkyl,
X is hydrogen,
Y is a radical selected from the group consisting of

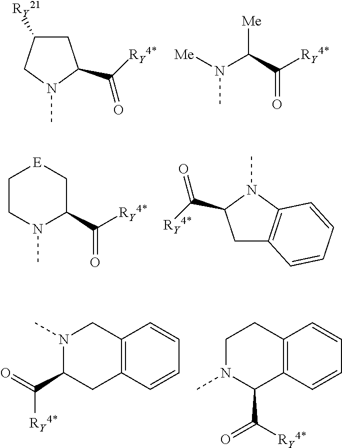

in which R_{Y}²¹ is selected independently of its respective occurrence from the group consisting of hydrogen, optionally substituted C₁-C₆-alkyl, optionally substituted C₃-C₇-cycloalkyl, OR_{Y}⁸, NR_{Y}⁹R_{Y}¹⁰, CONR_{Y}⁹R_{Y}¹⁰ and halogen; in which
   R_{Y}⁸, R_{Y}⁹ and R_{Y}¹⁰ are independently of one another and independently of their respective occurrence H, optionally substituted C₁-C₆-alkyl, optionally substituted C₁-C₇-cycloalkyl or optionally substituted phenyl,
   where
   R_{Y}⁸ may also independently of its respective occurrence be a radical —(CH₂)ₙ, —COR_{Y}¹⁵ or —CO—(CH₂)ₙ—CONR_{Y}¹⁶R_{Y}¹⁷,
   in which
   R_{Y}¹⁵ is independently of its respective occurrence H, OH, C₁-C₆-alkyl, C₁-C₆-alkoxy, C₁-C₆-cycloalkyl, CH₂CH₂COOH, NR_{Y}¹⁸R_{Y}¹⁸, preferably H, CH₃, C₂H₅, iso-C₃H₇, cyclohexyl, —CH₂CH₂COOH, NH₂, or N(CH₃)₂,
   R_{Y}¹⁶ and R_{Y}¹⁷ are independently of one another and independently of their respective occurrence H, C₁-C₆-alkyl or C₃-C₇-cycloalkyl,
   or R_{Y}¹⁶ and R_{Y}¹⁷ may also independently of their respective occurrence together form a ring selected from the group consisting of azetidin-1-yl, pyrrolidin-1-yl, piperidin-1-yl, piperazin-1-yl, morpholin-4-yl and thiomorpholin-4-yl;
   R_{Y}¹⁸ is independently of its respective occurrence H, C₁-C₆-alkyl or C₃-C₆-cycloalkyl,
   R_{Y}¹⁹ is independently of its respective occurrence H, C₁-C₆-alkyl, C₃-C₆-cycloalkyl, —C(CH₃)₂CH₂OH, —C(CH₃)(CH₂OH)₂ or —C(CH₂OH)₃;
   or R_{Y}¹⁸ and R_{Y}¹⁹ may also independently of their respective occurrence together form a ring selected from the group consisting of azetidin-1-yl, pyrrolidin-1-yl, piperidin-1-yl, piperazin-1-yl, morpholin-4-yl and thiomorpholin-4-yl;
   R_{Y}¹⁰ may also independently of its respective occurrence be a group COR_{Y}²⁰ in which R_{Y}²⁰ is hydrogen, optionally substituted C₁-C₄-alkyl or optionally substituted phenyl,
   where R_{Y}⁹ and R_{Y}¹⁰ may also independently of their respective occurrence together form a 5- or 6-membered, saturated or unsaturated carbocycle which may include a heteroatom selected from the group consisting of O, S and NR_{Y}¹⁴ as ring member, in which R_{Y}¹⁴ is hydrogen or C₁-C₄-alkyl,
   n is independently of its respective occurrence the integer 1 or 2;
   R_{Y}^{4*} is independently of its respective occurrence NHC2H5, N(CH3)₂, or azetidinyl,
   E is independently of its respective occurrence O or CH₂,
   in which
   the carbon which carries the radical R_{Y}²¹ has the (R) configuration,
   the carbon which carries the radical COR_{Y}^{4*} has the (S) configuration, and
      the carbon in position 3 of the indol-2-one may have the (R) or (S) configuration,
   the tautomeric, enantiomeric and diastereomeric forms thereof, and the prodrugs thereof, and the physiologically tolerated salts of said compounds.

Each of these aforementioned preferred definitions of a variable can be combined with any of the aforementioned preferred definitions of the remaining variables.

In a further preferred embodiment, at least one compound of the aforementioned general formula (I) is provided, characterized in that
A is the group

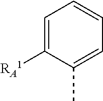

in which R_{A}¹ is selected from the group consisting of chlorine, methyl, ethyl, OCH₃, OC₂H₅, OC₃H₇, O-iso-C₃H₇, fluorine, CF₃ and OCF₃,
B is a phenyl ring which may be substituted by the radicals R_{B}¹ and R_{B}², where R_{B}¹ and R_{B}² are selected independently of one another from the group consisting of hydrogen, fluorine, chlorine, CN, CF₃, OCF₃, OCHF₂, O—C₁-C₄-alkyl, OH, C₁-C₆-alkyl and C₃-C₇-cycloalkyl,
X is hydrogen,
Y is a radical selected from the group consisting of the radicals Y1 to Y20 mentioned below:

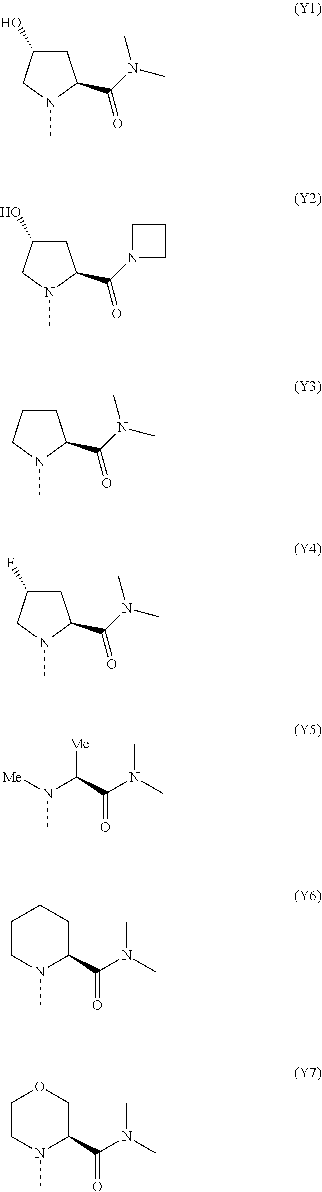

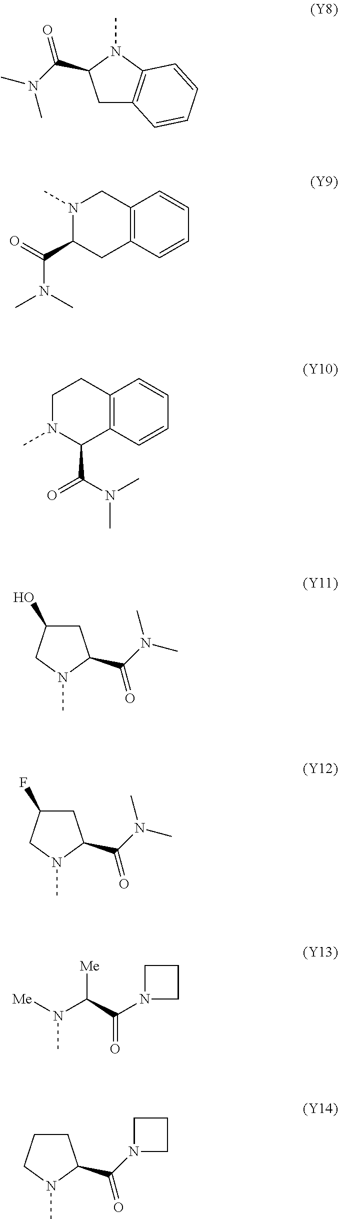

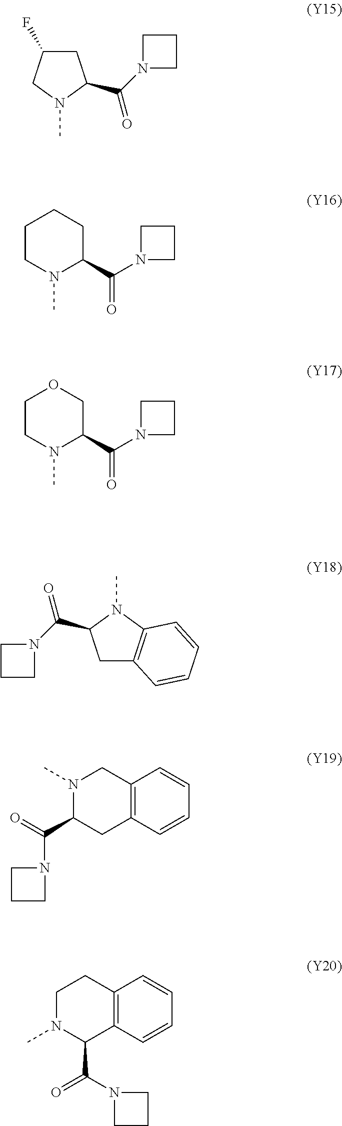

in which

the carbon which carries the radical hydroxy (OH) or fluorine (F) has the (R) or (S) configuration,

the carbon which carries the amide group has the (S) configuration, and the carbon in position 3 of the indol-2-one may have the (R) or (S) configuration,

the tautomeric, enantiomeric and diastereomeric forms thereof, and the prodrugs thereof, and the physiologically tolerated salts of said compounds.

Each of the aforementioned preferred definitions of a variable can be combined with any of the aforementioned preferred definitions of the remaining variables.

In a further preferred embodiment, at least one compound of the aforementioned general formula (I) are provided, characterized in that it rotates the plane of polarized light to the left, that is to say has a negative rotation.

In a further preferred embodiment, at least one compound of the aforementioned general formula (I) are provided, characterized in that it has a binding affinity Ki for a vasopressin V1b receptor subtype of less than 100 nM.

In a further preferred embodiment, at least one compound of the aforementioned general formula (I) are provided, characterized in that it has a selectivity for the vasopressin V1b receptor subtype vis-à-vis the vasopressin V1a receptor subtype, that is the quotient of Ki(V1a)/Ki(V1b) is at least greater than 1.

In a further preferred embodiment, at least one compound of the aforementioned general formula (I) are provided, characterized in that it has a selectivity for the vasopressin V1b receptor subtype vis-à-vis the vasopressin V2 receptor subtype, that is the quotient of Ki(V2)/Ki(V1b) is at least greater than 1.

In a further preferred embodiment, at least one compound of the aforementioned general formula (I) are provided, characterized in that it has a selectivity for the vasopressin V1b receptor subtype vis-à-vis the oxytocin (OT) receptor, that is the quotient of Ki(OT)/Ki(V1b) is at least greater than 1.

In a further preferred embodiment, at least one compound of the aforementioned general formula (I) are provided, characterized in that it has a binding affinity Ki for the vasopressin V1b receptor subtype of less than 100 nM and a selectivity for the vasopressin V1b receptor subtype vis-à-vis the vasopressin V1a receptor subtype, that is the quotient of Ki(V1a)/Ki(V1b) is at least greater than 1.

In a further preferred embodiment, at least one compound of the aforementioned general formula (I) are provided, characterized in that it has a binding affinity Ki for the vasopressin V1b receptor subtype of less than 100 nM and a selectivity for the vasopressin V1b receptor subtype vis-à-vis the vasopressin V2 receptor subtype, that is the quotient of Ki(V2)/Ki(V1b) is at least greater than 1.

In a further preferred embodiment, at least one compound of the aforementioned general formula (I) are provided, characterized in that it has a binding affinity Ki for the vasopressin V1b receptor subtype of less than 100 nM and a selectivity for the vasopressin V1b receptor subtype vis-à-vis the oxytocin (OT) receptor, that is the quotient of Ki(OT)/Ki(V1b) is at least greater than 1.

In a further preferred embodiment, at least one compound of the aforementioned general formula (I) are provided, characterized in that it has a binding affinity Ki for the vasopressin V1b receptor subtype of less than 100 nM and selectivities for the vasopressin V1b receptor subtype vis-à-vis the vasopressin V1a receptor subtype and the vasopressin V2 receptor subtype, that is the quotients of Ki(V1a)/Ki(V1b) and Ki(V2)/Ki(V1b) are in each case at least greater than 1.

In a further preferred embodiment, at least one compound of the aforementioned general formula (I) are provided, characterized in that it has a binding affinity Ki for the vasopressin V1b receptor subtype of less than 100 nM and simultaneous selectivities for the vasopressin V1b receptor subtype vis-à-vis the vasopressin V1a receptor subtype and the oxytocin (OT) receptor, that is the quotients of Ki(V1a)/Ki(V1b) and Ki(OT)/Ki(V1b) are in each case at least greater than 1.

In a further preferred embodiment, at least one compound of the aforementioned general formula (I) are provided, characterized in that it has a binding affinity Ki for the vasopressin V1b receptor subtype of less than 100 nM and simultaneous selectivities for the vasopressin V1b receptor subtype vis-à-vis the vasopressin V2 receptor subtype and the oxytocin (OT) receptor, that is the quotients of Ki(V2)/Ki(V1b) and Ki(OT)/Ki(V1b) are in each case at least greater than 1.

In a further preferred embodiment, at least one compound of the aforementioned general formula (I) is provided, characterized in that it has a binding affinity Ki for the vasopressin V1b receptor subtype of less than 100 nM and simultaneous selectivities for the vasopressin V1b receptor subtype vis-à-vis the vasopressin V1a receptor subtype, the vasopressin V2 receptor subtype and the oxytocin (OT) receptor, that is the quotients of Ki(V1a)/Ki(V1b), Ki(V2)/Ki(V1b) and Ki(OT)/Ki(V1b) are in each case at least greater than 1.

In a further aspect of the present invention, medicaments comprising at least one of the aforementioned compounds according to general formula (I) are provided.

In a further aspect of the present invention, at least one of the aforementioned compounds are provided for use as medicament.

In a further aspect of the present invention, the use of at least one of the aforementioned compounds according to general formula (I) is provided for the treatment and/or prophylaxis of at least one vasopressin-dependent and/or oxytocin-dependent disease and/or for the manufacture of a medicament for the treatment and/or prophylaxis of at least one of said diseases.

In a further aspect of the present invention, the use of at least one of the aforementioned compounds according to general formula (I) is provided for the treatment and/or prophylaxis of at least one disorder selected from the group consisting of diabetes insipidus, nocturnal enuresis, incontinence, diseases in which blood coagulation disorders occur, and/or for delaying micturition and/or for the manufacture of a medicament for the treatment and/or prophylaxis of at least one of said diseases.

In a further aspect of the present invention, the use of at least one of the aforementioned compounds according to general formula (I) is provided for the treatment and/or prophylaxis of at least one disorder selected from the group consisting of hypertension, pulmonary hypertension, heart failure, myocardial infarction, coronary spasm, unstable angina, PTCA (percutaneous transluminal coronary angioplastie), ischemias of the heart, disorders of the renal system, edemas, renal vasospasm, necrosis of the renal cortex, hyponatremia, hypokalemia, Schwartz-Bartter syndrome, disorders of the gastrointestinal tract, gastritic vasospasm, hepatocirrhosis, gastric and intestinal ulcer, emesis, emesis occurring during chemotherapy, and/or travel sickness and/or for the manufacture of a medicament for the treatment and/or prophylaxis of at least one of said diseases.

In a further aspect of the present invention, the use of at least one of the aforementioned compounds according to general formula (I) is provided for the treatment of affective disorders and/or for the manufacture of a medicament for the treatment of affective disorders.

In a further aspect of the present invention, the use of at least one of the aforementioned compounds according to general formula (I) is provided for the treatment of anxiety disorders and/or stress-dependent anxiety disorders and/or for the manufacture of a medicament for the treatment of anxiety disorders and/or stress-dependent anxiety disorders.

In a further aspect of the present invention, the use of at least one of the aforementioned compounds according to general formula (I) is provided for the treatment of memory impairments and/or Alzheimer's disease and/or for the manufacture of a medicament for the treatment of memory impairments and/or Alzheimer's disease.

In a further aspect of the present invention, the use of at least one of the aforementioned compounds according to general formula (I) is provided for the treatment of psychoses and/or psychotic disorders and/or for the manufacture of a medicament for the treatment of psychoses and/or psychotic disorders.

In a further aspect of the present invention, the use of at least one of the aforementioned compounds according to general formula (I) is provided for the treatment of Cushing's syndrome and/or for the manufacture of a medicament for the treatment of Cushing's syndrome.

In a further aspect of the present invention, the use of at least one of the aforementioned compounds according to general formula (I) is provided for the treatment of sleep disorders and/or for the manufacture of a medicament for the treatment of sleep disorders.

In a further aspect of the present invention, a method is provided for the treatment and/or prophylaxis of at least one disorder selected from the group consisting of diabetes insipidus, nocturnal enuresis, incontinence, diseases in which blood coagulation disorders occur, and for delaying micturition, in a patient, characterized in that an effective amount of at least one of the aforementioned compounds of the general formula (I) is administered to the patient.

In a further aspect of the present invention, a method is provided for the treatment and/or prophylaxis of at least one disorder selected from the group consisting of hypertension, pulmonary hypertension, heart failure, myocardial infarction, coronary spasm, unstable angina, PTCA (percutaneous transluminal coronary angioplastie), ischemias of the heart, disorders of the renal system, edemas, renal vasospasm, necrosis of the renal cortex, hyponatremia, hypokalemia, Schwartz-Bartter syndrome, disorders of the gastrointestinal tract, gastritic vasospasm, hepatocirrhosis, gastric and intestinal ulcer, emesis, emesis occurring during chemotherapy, and travel sickness, in a patient, characterized in that an effective amount of at least one of the aforementioned compounds of the general formula (I) is administered to the patient.

In a further aspect of the present invention, a method is provided for the treatment and/or prophylaxis of affective disorders in a patient, characterized in that an effective amount of at least one of the aforementioned compounds of the general formula (I) is administered to the patient.

In a further aspect of the present invention, a method is provided for the treatment of anxiety disorders and/or stress-dependent anxiety disorders in a patient, characterized in that an effective amount of at least one of the aforementioned compounds of the general formula (I) is administered to the patient.

In a further aspect of the present invention, a method is provided for the treatment of memory impairments and/or Alzheimer's disease in a patient, characterized in that an effective amount of at least one of the aforementioned compounds of the general formula (I) is administered to the patient.

In a further aspect of the present invention, a method is provided for the treatment of psychoses and/or psychotic disorders in a patient, characterized in that an effective amount of at least one of the aforementioned compounds of the general formula (I) is administered to the patient.

In a further aspect of the present invention, a method is provided for the treatment of Cushing's syndrome in a patient, characterized in that an effective amount of at least one of the aforementioned compounds of the general formula (I) is administered to the patient.

In a further aspect of the present invention, a method is provided for the treatment of sleep disorders in a patient, characterized in that an effective amount of at least one of the aforementioned compounds of the general formula (I) is administered to the patient.

The aforementioned patients are preferably mammals, particularly preferably humans and non-human mammals (non-human animals).

In a further aspect of the present invention, a method is provided for preparing at least one of the aforementioned compound of the general formula (I), characterized in that either an isatin derivative which is substituted in position 5 by a leaving or convertible radical which is suitable for replacement by or conversion into the cyano group, or a suitable 5-cyanoisatin derivative, is employed as starting material.

In a preferred embodiment, a method is provided for preparing at least one of the aforementioned compound of the general formula (I), characterized in that the cyano group is introduced into position 5 of the oxindole ring by replacement and conversion in the first or the last step of the method or a step of the method in between.

In a further embodiment, the following compounds of the aforementioned general formula (I) are particularly preferred:
(2S,4R)-1-[5-Cyano-1-(2,4-dimethoxybenzenesulfonyl)-3-(2-methoxyphenyl)-2-oxo-2,3-dihydro-1H-indol-3-yl]-4-hydroxypyrrolidine-2-dimethylcarboxamide;
(±)-(S)-1-[5-Cyano-1-(4-methoxybenzenesulfonyl)-3-(2-methoxyphenyl)-2-oxo-2,3-dihydro-1H-indol-3-yl]pyrrolidine-2-dimethylcarboxamide;
(+)-(S)-1-[5-Cyano-1-(2,4-dimethoxybenzenesulfonyl)-3-(2-methoxyphenyl)-2-oxo-2,3-dihydro-1H-indol-3-yl]pyrrolidine-2-dimethylcarboxamide;
(−)-(S)-1-[5-Cyano-1-(2,4-dimethoxybenzenesulfonyl)-3-(2-methoxyphenyl)-2-oxo-2,3-dihydro-1H-indol-3-yl]pyrrolidine-2-dimethylcarboxamide;
(±)-(S)-1-[5-Cyano-1-(2,4-dimethoxybenzenesulfonyl)-3-(2-methoxyphenyl)-2-oxo-2,3-dihydro-1H-indol-3-yl]piperidine-2-dimethylcarboxamide;
(+)-(S)-2-{[5-Cyano-1-(2,4-dimethoxybenzenesulfonyl)-3-(2-methoxyphenyl)-2-oxo-2,3-dihydro-1H-indol-3-yl]methylamino}-N,N-dimethylpropionamide;
(−)-(2S,4R)-1-[5-Cyano-1-(4-methoxybenzenesulfonyl)-3-(2-methoxyphenyl)-2-oxo-2,3-dihydro-1H-indol-3-yl]-4-hydroxypyrrolidine-2-dimethylcarboxamide;
(−)-(2S,4R)-1-[5-Cyano-1-(2,4-dichlorobenzenesulfonyl)-3-(2-methoxyphenyl)-2-oxo-2,3-dihydro-1H-indol-3-yl]-4-hydroxypyrrolidine-2-dimethylcarboxamide;
(−)-(S)-2-{[5-Cyano-1-(2,4-dimethoxybenzenesulfonyl)-3-(2-methoxyphenyl)-2-oxo-2,3-dihydro-1H-indol-3-yl]methylamino}-N,N-dimethylpropionamide;
(±)-(S)-1-[5-Cyano-1-(4-cyanobenzenesulfonyl)-3-(2-methoxyphenyl)-2-oxo-2,3-dihydro-1H-indol-3-yl]pyrrolidine-2-dimethylcarboxamide;
(±)-(S)-1-[5-Cyano-1-(4-ethylbenzenesulfonyl)-3-(2-methoxyphenyl)-2-oxo-2,3-dihydro-1H-indol-3-yl]pyrrolidine-2-dimethylcarboxamide;
(±)-(S)-2-{[1-(4-Chlorobenzenesulfonyl)-5-cyano-3-(2-methoxyphenyl)-2-oxo-2,3-dihydro-1H-indol-3-yl]methylamino}-N,N-dimethylpropionamide;
(±)-(S)-2-{[5-Cyano-3-(2-methoxyphenyl)-2-oxo-1-(4-trifluoromethoxy-benzenesulfonyl)-2,3-dihydro-1H-indol-3-yl]methylamino}-N,N-dimethyl-propionamide;
(±)-(S)-2-{[5-Cyano-1-(4-isopropylbenzenesulfonyl)-3-(2-methoxyphenyl)-2-oxo-2,3-dihydro-1H-indol-3-yl]methylamino}-N,N-dimethylpropionamide;
(±)-(S)-2-{[5-Cyano-1-(4-methoxybenzenesulfonyl)-3-(2-methoxyphenyl)-2-oxo-2,3-dihydro-1H-indol-3-yl]methylamino}-N,N-dimethylpropionamide;
(+)-(2S,4R)-1-[5-Cyano-1-(2,4-dimethoxybenzenesulfonyl)-3-(2-methoxyphenyl)-2-oxo-2,3-dihydro-1H-indol-3-yl]-4-hydroxypyrrolidine-2-dimethylcarboxamide;
(−)-(2S)-1-[5-Cyano-1-(4-methoxybenzenesulfonyl)-3-(2-methoxyphenyl)-2-oxo-2,3-dihydro-1H-indol-3-yl]pyrrolidine-2-dimethylcarboxamide;
(+)-(2S)-1-[5-Cyano-1-(4-methoxybenzenesulfonyl)-3-(2-methoxyphenyl)-2-oxo-2,3-dihydro-1H-indol-3-yl]pyrrolidine-2-dimethylcarboxamide;
(−)-(2S)-1-[5-Cyano-1-(2,4-dimethoxybenzenesulfonyl)-3-(2-methoxyphenyl)-2-oxo-2,3-dihydro-1H-indol-3-yl]piperidine-2-dimethylcarboxamide;
(+)-(2S)-1-[5-Cyano-1-(2,4-dimethoxybenzenesulfonyl)-3-(2-methoxyphenyl)-2-oxo-2,3-dihydro-1H-indol-3-yl]piperidine-2-dimethylcarboxamide;
(−)-(2S,4R)-1-[5-Cyano-1-(4-methoxybenzenesulfonyl)-3-(2-methoxyphenyl)-2-oxo-2,3-dihydro-1H-indol-3-yl]-4-hydroxypyrrolidine-2-dimethylcarboxamide;
(−)-(2S)-1-[5-Cyano-1-(4-ethylbenzenesulfonyl)-3-(2-methoxyphenyl)-2-oxo-2,3-dihydro-1H-indol-3-yl]pyrrolidine-2-dimethylcarboxamide;
(+)-(2S)-1-[5-Cyano-1-(4-ethylbenzenesulfonyl)-3-(2-methoxyphenyl)-2-oxo-2,3-dihydro-1H-indol-3-yl]pyrrolidine-2-dimethylcarboxamide;
   and the tautomeric, enantiomeric and diastereomeric forms thereof, and the prodrugs thereof, and non-salt forms and other physiologically tolerated salts of the aforementioned compounds.

The compounds of the invention may be in the form of racemates or of enantiopure or diastereopure compounds. The compounds are preferably in the form of enantiopure or diastereopure compounds.

Physiologically tolerated salts can be formed for example with the following anions:

chloride, bromide, phosphate, carbonate, nitrate, perchlorate, sulfate, citrate, lactate, tartrate, maleate, fumarate, mandelate, benzoate, ascorbate, cinnamate, glycollate, methanesulfonate, formate, malonate, naphthalene-2-sulfonate, tosylates, salicylate and/or acetate. Further suitable acids are listed for example in “Fortschritte der Arzneimittelforschung”, 1966, Birkhäuser Verlag, vol. 10, pp. 224-285.

In the context of the present invention, the terms “alkyl” or “alkylene” always comprise unbranched or branched “alkyl” or “alkylene”.

C₁-C₄-Alkyl is in the context of the description preferably methyl, ethyl, n-propyl, i-propyl, n-butyl, sec-butyl or t-butyl.

C₀-Alkylene or (CH₂)₀ designate in the context of the description a single bond or hydrogen.

The terms alkyl, C₁-C₆-alkyl, C₁-C₅-alkyl and C₁-C₄-alkyl mean in the context of the description a straight-chain or branched saturated hydrocarbon chain having the number of carbon atoms indicated in each case, preferably 1 to 6, more preferably 1 to 4, carbon atoms, such as, for example, methyl, ethyl, propyl, 1-methylethyl, butyl, 1-methylpropyl, 2-methylpropyl, 1,1-dimethylethyl, pentyl, 1-methylbutyl, 2-methylbutyl, 1,2-dimethylpropyl, 1,1-dimethylpropyl, 2,2-dimethylpropyl, 1-ethylpropyl, n-hexyl, 1-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl or 1-ethyl-2-methylpropyl, preferably methyl, ethyl, propyl, n-butyl or i-butyl.

The terms alkylene, C₁-C₆-alkylene and C₁-C₄-alkylene mean in the context of the description an alkyl group as defined above in which one hydrogen atom is replaced by a bond. Examples which are to be particularly mentioned are methylene, eth-1,2-ylene, prop-1,2-ylene, prop-1,3-ylene, but-1,2-ylene, but-1,3-ylene, but-2,3-ylene, but-1,4-ylene, 2-methylprop-1,3-ylene, pent-1,2-ylene, pent-1,3-ylene, pent-1,4-ylene, pent-1,5-ylene, pent-2,3-ylene, pent-2,4-ylene, 1-methylbut-1,4-ylene, 2-methylbut-1,4-ylene, 2-methylbut-1,3-ylene, 2-ethylprop-1,3-ylene, hex-3,4-ylene, 3-methylpent-2,4-ylene, hept-3,5-ylene, 2-ethylpent-1,3-ylene, 3-ethylhept-3,5-ylene, etc., preferably methylene, eth-1,2-ylene and prop-1,2-ylene.

The terms aryl, C₆-C₂₀-aryl and C₆-C₁₀-aryl mean in each case in the context of the description an aromatic mono-, bi- or polycyclic radical having, preferably, 6 to 20 carbon atoms, more preferably 6 to 10 carbon atoms, and is preferably selected from phenyl, biphenyl, naphthyl, tetrahydronaphthyl, fluorenyl, indenyl and phenanthrenyl, more preferably from phenyl and naphthyl, such as 1-naphthyl or 2-naphthyl. Phenyl is most preferred.

The terms hetaryl, C₆-C₂₀-hetaryl, C₆-C₁₀-hetaryl, C₁-C₁₀-hetaryl, C₂-C₁₀-hetaryl, C₃-C₁₀-hetaryl, C₁-C₆-hetaryl and C₁-C₅-hetaryl mean, unless stated otherwise, in the context of the description an aromatic ring comprising at least one heteroatom, preferably 1 or 2 heteroatoms, selected from the group of O, N or S and 1 to 10, preferably 2 to 10, more preferably 3 to 10, particularly preferably 1 to 6, even more particularly preferably 1 to 5 carbon atoms. The aromatic ring is preferably 5- or 6-membered. Hetaryl additionally comprises the derivatives thereof fused to aryl, specifically an aromatic radical having, preferably, 6 to 20 carbon atoms, more preferably 6 to 10 carbon atoms, most preferably phenyl, which is fused to this aromatic ring comprising at least one heteroatom. Hetaryl may also be selected from an aromatic radical having, preferably, 6 to 20, more preferably 6 to 10 carbon atoms, most preferably phenyl, with a heterocycloalkyl group which is fused thereto. In this connection, the heterocycloalkyl group is as defined above. Hetaryl is preferably selected from 2-furyl, 3-furyl, 2-pyrrolyl, 3-pyrrolyl, 2-thienyl, 3-thienyl, 2-pyridyl, 3-pyridyl, 4-pyridyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 3-isoxazolyl, 4-isoxazolyl, 5-isoxazolyl, thiadiazolyl, oxadiazolyl, triazinyl, indolinyl, benzothienyl, naphthothienyl, benzofuranyl, chromenyl, indolyl, isoindolyl, indazolyl, quinolyl, isoquinolyl, phthalazinyl, quinoxalinyl, benzimidazolyl and benzoxazolyl, 2,3-dihydro-1,4-benzodioxinyl, 1,3-benzodioxolyl-, 2,1,3-benzothiadiazolyl.

The terms cycloalkyl, C₃-C₇-cycloalkyl and C₃-C₆-cycloalkyl mean in the context of the description a saturated hydrocarbon ring having 3 to 7, preferably 3 to 6, carbon atoms, such as cyclopropyl, cyclobutyl, cyclopentyl, cyclohexyl or cycloheptyl.

C₃-C₇-Cycloalkenyl is in the context of the description a C₃-C₇-cycloalkyl as defined above which has one, two, three, four or more double bonds.

C₃-C₇-Heterocycloalkyl is in the context of the description a C₃-C₇-cycloalkyl as defined above having 1, 2, 3 or 4 identical or different heteroatoms selected from the group consisting of N, O and S.

C₃-C₇-Heterocycloalkenyl is in the context of the description a C₃-C₇-cycloalkenyl as defined above having 1, 2, 3 or 4 identical or different heteroatoms selected from the group consisting of N, O and S.

C₁-C₆-Haloalkyl is in the context of the description a C₁-C₆-alkyl as defined above in which one, more than one or all hydrogen atoms have been replaced by identical or different halogen atoms as defined below.

C₁-C₆-Haloalkoxy is in the context of the description a C₁-C₆-alkoxy as defined above in which one, more than one or all hydrogen atoms have been replaced by identical or different halogen atoms as defined below.

The terms acyl and C₁-C₆-acyl mean in the context of the description a straight-chain or branched radical —C(═O)—X, where unsubstituted or substituted radical may mean C₁-C₅-alkyl, C₂-C₅-alkenyl or C₂-C₅-alkynyl which are as defined above.

The terms alkenyl, C₂-C₆-alkenyl, C₂-C₅-alkenyl and C₂-C₄-alkenyl mean in the context of the description a branched or unbranched hydrocarbon chain comprising at least one double bond, having 2 to 6, preferably 2 to 4 carbon atoms. Alkenyl preferably comprises one or two double bonds, most preferably one double bond. Examples of alkenyl groups are those mentioned above for alkyl, with these groups comprising one or two double bonds, such as, for example, vinyl, 2-propenyl, 2-butenyl, 3-butenyl, 1-methyl-2-propenyl, 2-methyl-2-propenyl, 2-pentenyl, 3-pentenyl, 4-pentenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-2-propenyl, 1-ethyl-2-propenyl, 2-hexenyl, 3-hexenyl, 4-hexenyl, 5-hexenyl, 1-methyl-2-pentenyl, 2-methyl-2-pentenyl, 3-methyl-2-pentenyl, 4-methyl-2-pentenyl, 3-methyl-3-pentenyl, 4-methyl-3-pentenyl, 1-methyl-4-pentenyl, 2-methyl-4-pentenyl, 3-methyl-4-entenyl, 4-methyl-4-pentenyl, 1,1-dimethyl-2-butenyl, 1,1-dimethyl-3-butenyl, 1,2-dimethyl-2-butenyl, 1,2-dimethyl-3-butenyl, 1,3-dimethyl-2-butenyl, 1,3-dimethyl-3-butenyl, 2,2-dimethyl-3-butenyl, 2,3-dimethyl-2-butenyl, 2,3-dimethyl-3-butenyl, 1-ethyl-2-butenyl, 1-ethyl-3-butenyl, 2-ethyl-2-butenyl, 2-ethyl-3-butenyl, 1,1,2-trimethyl-2-propenyl, 1-ethyl-1-methyl-2-propenyl and 1-ethyl-2-methyl-2-propenyl, in particular 2-propenyl, 2-butenyl, 3-methyl-2-butenyl or 3-methyl-2-pentenyl.

The terms alkynyl, C₂-C₆-alkynyl, C₂-C₅-alkynyl and C₂-C₄-alkynyl mean in the context of the description a branched or unbranched hydrocarbon chain comprising at least one triple bond having 2 to 6, preferably 2 to 4, carbon atoms. Alkynyl preferably comprises one or two triple bonds, most preferably one triple bond. Examples of alkynyl groups are those mentioned above for alkyl, with these groups comprising one or two triple bonds, such as, for example, ethynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 1-methyl-2-pentynyl, 1-methyl-2-pentynyl, 1-methyl-3-pentynyl, 1-methyl-4-pentynyl, 2-methyl-3-pentynyl, 2-methyl-4-pentynyl, 3-methyl-4-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 1-ethyl-2-butynyl, 1-ethyl-3-butynyl, 2-ethyl-3-butynyl and 1-ethyl-1-methyl-2-propynyl, preferably ethynyl, 2-propynyl, 2-butynyl, 1-methyl-2-propynyl or 1-methyl-2-butynyl.

C₂-C₆-Alkenyloxy is in the context of the description is a C₂-C₆-alkenyl which is as defined above and is linked via oxygen.

C₂-C₆-Alkynyloxy is in the context of the description is in the context of the description is a C₂-C₆-alkynyl which is as defined above and is linked via oxygen.

The terms alkylthio, C₁-C₆-alkylthio, C₁-C₄-alkylthio and C₁-C₂-alkylthio mean in the context of the description a straight-chain or branched alkylenesulfanyl chain which comprises 1 to 6 carbon atoms and one sulfur atom. The alkylene radical preferably comprises 1 to 4, more preferably 1 or 2 carbon atoms, with alkylene being as defined above. Examples of thioalkyl include thiomethyl or thio-tert-butyl.

C₁-C₆-Alkylamino is in the context of the description is a C₁-C₆-alkyl which is as defined above and is linked via nitrogen.

C₁-C₆-Acylamino is in the context of the description a C₁-C₆-acyl which is as defined above and is linked via nitrogen.

Alkylenearyl is an aryl which is linked via C₁-C₆-, more preferably C₁-C₄-alkylene and is optionally substituted in the aryl radical, with alkylene and aryl being as defined above. Alkylenearyl is in particular benzyl or phenethyl which are optionally substituted in the aryl radical.

Aryloxy or —O-aryl is an aryl which is linked via oxygen and is as defined above, in particular —O-phenyl.

The term 3- to 10-membered carbocycle means in the context of the description a saturated or partly unsaturated hydrocarbon ring having 3 to 10 carbon atoms, such as, for example, cyclopropyl, cyclobutyl, cyclopentyl, cyclohexyl, cycloheptyl, cyclooctyl, cyclononyl or cyclodecanyl.

Alkylenearyl is an aryl which is linked via C₁-C₆-, more preferably C₁-C₄-alkylene and is optionally substituted in the aryl radical, with alkylene and aryl being as defined above. Alkylenearyl is in particular benzyl or phenethyl which is optionally substituted in the aryl radical.

The terms aryloxy, C₁-C₆-aryloxy or —O-aryl mean in the context of the description an aryl which is linked via oxygen and is as defined above, in particular —O-phenyl.

Alkylenehetaryl is a hetaryl which is linked via C₁-C₆-, more preferably C₁-C₄-alkylene and is optionally substituted in the hetaryl radical, with alkylene and hetaryl being as defined herein. Alkylenehetaryl is preferably optionally substituted —CH₂-2-pyridyl, —CH₂-3-pyridyl, —CH₂-4-pyridyl, —CH₂-2-thienyl, —CH₂-3-thienyl, —CH₂-4-thiazolyl, CH₂-5-thiazolyl, —CH₂—CH₂-2-pyridyl, —CH₂—CH₂-3-pyridyl, —CH₂—CH₂-4-pyridyl, —CH₂—CH₂-2-thienyl, —CH₂—CH₂-3-thienyl, —CH₂—CH₂-2-thiazolyl, —CH₂—CH₂-4-thiazolyl or —CH₂—CH₂-5-thiazolyl.

A bi- or tricyclic, saturated hydrocarbon radical is a bicycloalkyl or tricycloalkyl radical and has 5 to 18 carbon atoms. In the case of a bicycloalkyl radical, the ring system comprises preferably 5 to 12, more preferably 6 to 10, carbon atoms. In the case of a tricycloalkyl radical, the ring system comprises preferably 6 to 16, more preferably 6 to 12, carbon atoms. Examples of a bicycloalkyl radical include indanyl, camphyl and norbornyl. Examples of a tricycloalkyl radical include adamantyl.

Halogen is a halogen atom selected from fluorine, chlorine, bromine or iodine, preferably fluorine, chlorine or bromine, more preferably fluorine or chlorine.

Halogen-substituted alkyl designates an alkyl radical as defined above which is partially or completely substituted by fluorine, chlorine, bromine and/or iodine, thus, for example, CH₂F, CHF₂, CH₂Cl, 2-fluoroethyl, 2-chloroethyl, 2,2,2-trifluoroethyl.

The expression “substituted C₁-C₄-alkyl” in the context of the present invention means that some or all hydrogen atoms of the radical “C₁-C₄-alkyl” have been replaced by identical, different or partly identical and partly different substituents other than hydrogen. The maximum possible number of substituents is predetermined by the number of hydrogen atoms. The preferred number of substituents is one, two, three or four substituents. Preferred substituents are halogen, C₁-C₆-alkyl, O—C₁-C₆-alkyl, C₃-C₇-cycloalkyl, C₁-C₆-haloalkyl, O—C₁-C₆-haloalkyl or C₆-C₁₀-aryl.

Analogous statements to those made above about the expression “substituted C₁-C₄-alkyl” are also intended to apply to the expressions “substituted C₃-C₆-cycloalkyl”, “substituted phenyl”.

If mentioned, the radicals and groups may preferably be substituted one or more times, more preferably one, two or three times, most preferably once or twice. The expression “in each case optionally substituted” is intended to make it clear that not just the radical directly following thereon but all radicals mentioned in the respective group may be substituted.

Examples of substituents include: halogen, CN, CF₃, CHF₂, OCF₃, OCHF₂, NO₂, NH₂, OH, COOH, in each case branched or unbranched, optionally substituted C₁-C₆-alkyl, C₃-C₇-cycloalkyl, C₁-C₆-alkylene-O—C₁-C₆-alkyl or C₁-C₆-thioalkyl, O—C₁-C₄-alkyl, N(C₁-C₄-alkyl)₂, NH(C₁-C₄-alkyl), aryl, —O-aryl, C₁-C₄-alkylene-O-aryl, NHCO—C₁-C₄-alkyl, NH—SO₂—C₁-C₄-alkyl, CO—C₁₋₄-alkyl, SO₂—C₁-C₄-alkyl, NHSO₂-aryl, NHCO-aryl optionally substituted in the aryl radical, CONH₂, SO₂NH₂, SO₂-aryl, SO—C₁-C₄-alkyl, SO-aryl, N-pyrrolidinyl, N-piperidinyl, and N-morpholinyl. Preferred substituents are F, Cl, CF₃, OCF₃, NH₂, NO₂, OH, COOH, C₁-C₄-alkyl, methoxy, acetyl, NH-acetyl and SO₂NH₂.

Expressions in parentheses with subscript integers are to be understood in the context of the description in such a way that the meanings of the radicals in parentheses may in each case be identical or different. For example, N(C₁-C₄-alkyl)₂ in the context of the description stands for N(C₁-C₄-alkyl)(C₁-C₄-alkyl), it being possible for the two radicals (C₁-C₄-alkyl) to be identical or different.

The symbol ( - - - ) in the chemical formulae of Y and A depicts the points of linkage of respectively Y and A to the 3 position of the oxindole ring structure.

The expressions “compounds” and “at least one compound” are equivalent in the context of the invention and are intended to mean that one or more of said compounds may be involved.

The compounds of the invention are effective after administration by various routes (for example intravenously, intramuscularly, orally), especially orally.

The compounds of the invention show good affinity for vasopressin receptors, for example the vasopressin V1a and V1b receptor subtypes. Since the various vasopressin receptors mediate very different effects of vasopressin (M. Thibonnier, Exp. Opin. Invest. Drugs 1998, 7(5), 729-740; Serradeil-Le Gal, C, et al.; Prog Brain Res. 2002; 139:197-210), it is particularly important to obtain effects selectively on, for example, one vasopressin receptor, in order thus to achieve the desired effect without simultaneously causing considerable side effects. Thus, vasopressin mediates for example effects on the kidney and its function via the V2 receptor, and this would be unwanted during a possible treatment of CNS disorders. Accordingly, besides the actual affinity for the target receptor, also particularly important is the selectivity vis-à-vis the other vasopressin receptors. The compounds of the invention show the advantage of having very good affinities for the desired receptors such as the vasopressin V1b and V1a receptors and simultaneously displaying an improved selectivity vis-à-vis the other receptors such as V2.

The present invention also provides the use of the compounds of the invention for the treatment and/or prophylaxis of diseases in which the course of the disease is at least partially dependent on vasopressin, i.e. diseases which show an elevated vasopressin or oxytocin level which may contribute indirectly or indirectly to the pathological state.

The present invention further provides the use of the compounds of the invention for the treatment and/or prophylaxis of diseases such as, for example, diabetes insipidus, nocturnal enuresis, incontinence, diseases in which blood coagulation disorders occur and/or for delaying micturition.

The present invention also provides the use of the compounds of the invention for the treatment and/or prophylaxis of the following diseases: hypertension, pulmonary hypertension, heart failure, myocardial infarction, coronary spasm, unstable angina, PTCA (percutaneous transluminal coronary angioplasie), ischemias of the heart, disorders of the renal system, edemas, renal vasospasm, necrosis of the renal cortex, hyponatremia, hypokalemia, Schwartz-Bartter syndrome, disorders of the gastrointestinal tract, gastritic vasospasm, hepatocirrhosis, gastric and intestinal ulcer, emesis, emesis occurring during chemotherapy, and travel sickness.

The compounds of the invention can also be used for the treatment of various vasopressin-dependent or oxytocin-dependent complaints which have central nervous causes or causes in the HPA axis (hypothalamic pituitary adrenal axis), for example for affective disorders such as depressive disorders and bipolar disorders. These include for example dythymic disorders, phobias, post-traumatic stress disorders, general anxiety disorders, panic disorders, seasonal depressions and sleep disorders.

The compounds of the invention can likewise be employed for treatment in cases of anxiety disorders and stress-dependent anxiety disorders such as, for example, generalized anxiety disorders, phobias, post-traumatic anxiety disorders, panic anxiety disorders, obsessive-compulsive anxiety disorders, acute stress-dependent anxiety disorders and social phobia. The inventive compounds can further be employed also for the treatment of memory impairments, Alzheimer's disease, psychoses, psychotic disorders, sleep disorders and/or Cushing's syndrome.

The present invention also relates to pharmaceutical compositions which comprise an effective dose of a compound of the invention or of a pharmaceutically acceptable salt thereof and suitable pharmaceutical carriers.

These pharmaceutical carriers are chosen according to the pharmaceutical form and the desired mode of administration.

The compounds of the invention of the general formula I or optionally suitable salts of these compounds can be used to produce pharmaceutical compositions for oral, sublingual, subcutaneous, intramuscular, intravenous, topical, intratracheal, intranasal, transdermal or rectal administration and be administered to animals or humans in standard administration forms, mixed with conventional pharmaceutical carriers, for the prophylaxis or treatment of the above disorders or diseases.

The suitable standard administration forms comprise forms for oral administration, such as tablets, gelatin capsules, powders, granules and solutions or suspensions for oral intake, forms for sublingual, buccal, intratracheal or intranaseal administration, aerosols, implants, forms of subcutaneous, intramuscular or intravenous administration and forms of rectal administration.

The compounds of the invention can be used in creams, ointments or lotions for topical administration.

In order to achieve the desired prophylactic or therapeutic effect, the dose of the active basic ingredient can vary between 0.01 and 50 mg per kg of body weight and per day.

Each unit dose may comprise from 0.05 to 5000 mg, preferably 1 to 1000 mg, of the active ingredient in combination with a pharmaceutical carrier. This unit dose may be administered 1 to 5 times a day, so that a daily dose of from 0.5 to 25 000 mg, preferably 1 to 5000 mg, is administered.

If a solid composition is prepared in the form of tablets, the main ingredient is mixed with a pharmaceutical carrier such as gelatin, starch, lactose, magnesium stearate, talc, silicon dioxide or the like.

The tablets can be coated with sucrose, a cellulose derivative or another suitable substance, or be treated otherwise in order to display a sustained or delayed activity and in order to release a predetermined amount of the active basic ingredient continuously.

A preparation in the form of gelatin capsules is obtained by mixing the active ingredient with an extender and including the resulting mixture in soft or hard gelatin capsules.

A preparation in the form of a syrup or elixir or for administration in the form of drops may comprise active ingredients together with a sweetener, which is preferably calorie-free, methylparaben or propylparaben as antiseptics, a flavoring and a suitable color.

Water-dispersible powders or granules may comprise the active ingredients mixed with dispersants, wetting agents or suspending agents, such as polyvinylpyrrolidones, and sweeteners or masking flavors.

Rectal administration is achieved by using suppositories which are prepared with binders which melt at the rectal temperature, for example cocoa butter or polyethylene glycols. Parenteral administration is effected by using aqueous suspensions, isotonic saline solutions or sterile and injectable solutions which comprise pharmacologically acceptable dispersants and/or wetting agents, for example propylene glycol or polyethylene glycol.

The active basic ingredient may also be formulated as microcapsules or centrosomes, where suitable with one or more carriers or additives.

In addition to the compounds of the general formula (I) or their pharmaceutically acceptable salts, the compositions of the invention may comprise other active basic ingredients which may be beneficial for the treatment of the disorders or diseases indicated above.

The present invention thus further relates to pharmaceutical compositions in which a plurality of active basic ingredients are present together, at least one of these being a compound of the invention.

The compounds of the invention represent antagonists of the so-called receptors of the vasopressin/oxytocin family. Such compounds can be investigated in suitable assays which ascertain the affinity for a receptor, where the affinity constant Ki represents a measure of the potency of the compounds and a smaller value represents a greater potency. The compounds of the invention have been tested for example for their receptor affinity in relation to the vasopressin V1b, V1a, V2 receptor subtypes and/or the oxytocin receptor.

### Preparation of the Compounds of the Invention

Exemplary synthetic routes for preparing the compounds of the invention are described below.

The oxindoles of the invention can be prepared for example by the route outlined in synthesis schemes 1. The variables in synthesis scheme 1 have the same meanings as in the general formula (I)

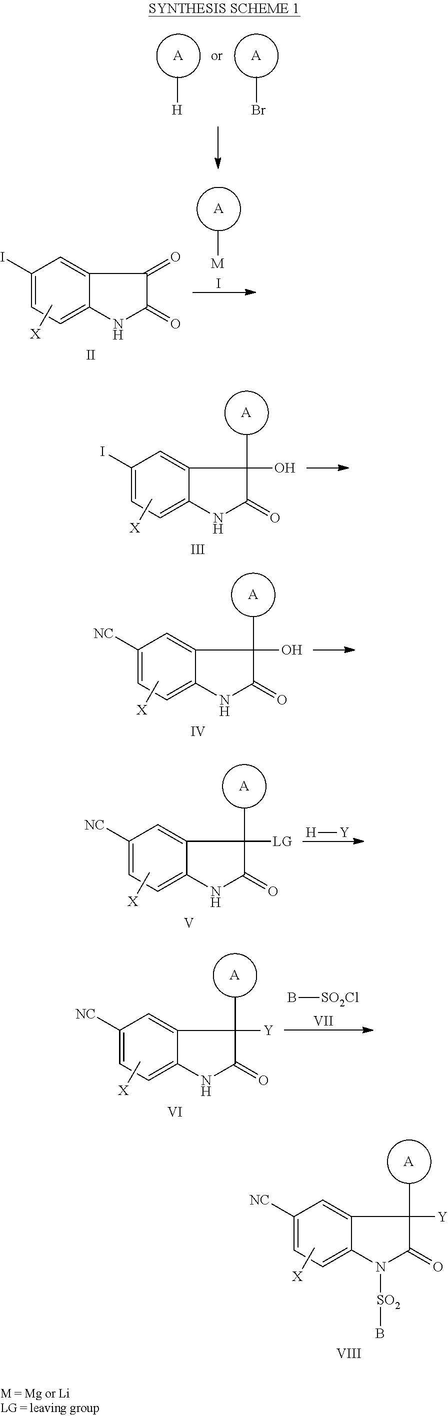

Starting from compounds A-H or A-Br or A-Cl (2-methoxypyridine is mentioned by way of example in synthesis schemes 2), which are metallated in a conventional way, such as, for example, as Grignard compound (Mg) or organyllithium compound (as in scheme 1), the 3-hydroxyoxindoles can be obtained by addition to isatins) 5-iodoisatin is mentioned by way of example in scheme 1). The metallated compounds can be obtained in a conventional way from halogen compounds or hydrocarbon compounds. Examples of methods are present in Houben-Weil, Methoden zur Organischen Chemie, vol. 13, 1-2, Chap. Mg— and Li compounds. The isatins II are either commercially available or were prepared in analogy to methods described in the literature (Advances in Heterocyclic Chemistry, A. R. Katritzky and A. J. Boulton, Academic Press, New York, 1975, 18, 2-58; J. Brazil. Chem. Soc. 12, 273-324, 2001).

Replacement of the 5-iodo substituents to obtain the corresponding 5-cyano compounds takes place by procedures known per se, as described for example in J. Org. Chem. (1998), 63(23), 8224-8, J. Org. Chem. (1997), 62(25), 8634-9, J. Label. Cpd Rad. (1994), 34(9), 887-97 and J. Med. Chem. 1995, 38, 745-52. In scheme I, for example, the exchange takes place by using the reagents Zn(CN)₂ and [[C₆H₅)₃P]₄Pd in dimethylformamide (DMF) as solvent.

The 3-hydroxyoxindoles (III) can be converted into the compounds (V) which have a leaving group (LG) in position 3, it being possible for the leaving group (LG) to be conventional leaving groups such as, for example, halides, mesylate or tosylate. Thus, for example (LG=chlorine), the intermediate (V) can be prepared by treating the alcohol (IV) with thionyl chloride in the presence of a base such as, for example, pyridine. Alternatively, alcohols (IV) can be obtained by conversion into the mesylate using methanesulfonyl chloride in the presence of a base such as, for example, triethylamine. The compounds (V) are subsequently reacted with suitable amines (for example in synthesis schemes 2 with (2S,4R)-4-hydroxypyrrolidine-2-dimethyl-carboxamide hydrochloride), resulting in the analogous amine compounds (VI). For example, such substitution reactions with amines in the presence of a base such as N,N-diisopropylethylamine can provide the analogous 3-aminooxindoles (VI). The amine compound (VI) obtained in this way can then be converted by treatment with sulfonyl chlorides R′-SO2Cl after deprotonation with a strong base such as, for example, potassium tert-butoxide or sodium hydride, in DMF, into the corresponding sulfone compound (VII).

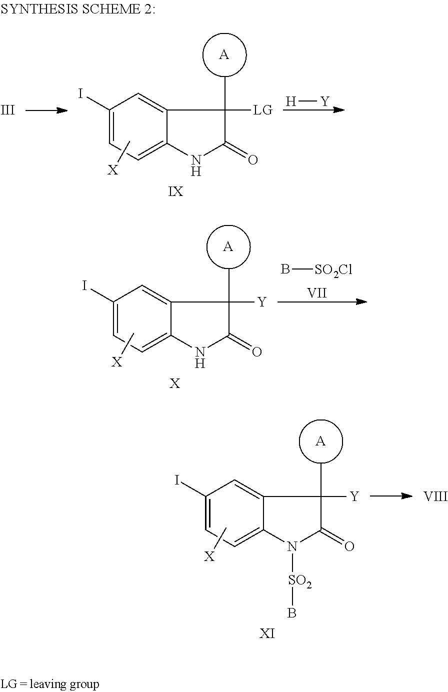

Alternatively, introduction of the 5-cyano group can also take place in a later synthesis step, for example by exchanging the 5-iodo substituent in compound (X) to obtain the corresponding 5-cyano compound (VI) by procedures known per se (as described above for example). Alternatively, the exchange of iodine for cyano in position 5 can also take place at the stage of compound (XI) to result in compound (VIII) (see synthesis scheme 2).

The invention is explained in more detail below by means of examples without being restricted to the examples.

### EXPERIMENTAL SECTION

### Example 1

### (2S,4R)-1-[5-Cyano-1-(2,4-dimethoxybenzenesulfonyl)-3-(2-methoxyphenyl)-2-oxo-2,3-dihydro-1H-indol-3-yl]-4-hydroxypyrrolidine-2-dimethylcarboxamide

### A) 3-Hydroxy-5-iodo-3-(2-methoxyphenyl)-1,3-dihydroindol-2-one

A solution of 2-methoxyphenylmagnesium bromide in THF (56 mmol, 56 ml, 1.0 M) was added dropwise to a suspension of 5-iodoisatin (22 mmol, 6.00 g) in THF (70 ml) while cooling in ice. Addition was followed by stirring at room temperature for 1 hour. The reaction mixture was quenched by adding ammonium chloride solution and extracted several times with ethyl acetate. The combined organic phase was washed several times with water, dried over MgSO₄ and concentrated under reduced pressure. The desired product starts to crystallize out during concentration. After storage in a refrigerator overnight, the precipitate was filtered off with suction, washed with ethyl acetate and dried. 5.6 g (67%) of the desired product were obtained.

Mass spectrum: m/z=364 [M+H—H₂O]

### B) (2S,4R)-4-Hydroxy-1-[5-iodo-3-(2-methoxyphenyl)-2-oxo-2,3-dihydro-1H-indol-3-yl]-pyrrolidine-2-dimethylcarboxamide

Pyridine (14 mmol, 1.14 ml) and thionyl chloride (0.82 ml) were successively added dropwise to an ice-cold solution of example 1A (8 mmol, 3.05 g) in dichloromethane (80 ml). The reaction mixture was stirred at room temperature overnight and then water was added. The organic phase was separated off and the aqueous phase was extracted once more with dichloromethane. The combined organic phase was washed several times with water, dried over MgSO₄ and concentrated under reduced pressure. The residue was dissolved in dichloromethane (20 ml), tetrahydrofuran (4 ml) and diisopropylethylamine (Hünig's base, 3 ml), and (2S,4R)-4-hydroxypyrrolidine-2-dimethylcarboxamide hydrochloride (7 mmol, 1.37 g, WO 01/55130) was added to this solution. The reaction mixture was stirred at room temperature overnight. The mixture was concentrated under reduced pressure and, after addition of water, extracted several times with ethyl acetate. Separation by chromatography on silica gel (gradient: 4% to 8% MeOH in dichloromethane) afforded the two diastereomeric products:

Less polar (faster-eluting) diastereomer: 400 mg, m/z=522 [M+H]

More polar (slower-eluting) diastereomer: 810 mg, m/z=522 [M+H]

### C) (2S,4R)-1-[1-(2,4-Dimethoxybenzenesulfonyl)-5-iodo-3-(2-methoxyphenyl)-2-oxo-2,3-dihydro-1H-indol-3-yl]-4-hydroxypyrrolidine-2-dimethylcarboxamide; levorotatory diastereomer

Sodium hydride (0.8 mmol, 32 mg of a 60% dispersion in mineral oil) was added to an ice-cold solution of example 1B (more polar diastereomer) (0.77 mmol, 400 mg) in DMF (4 ml), and the mixture was stirred at 0° C. for 30 min. After addition of 2,4-dimethoxyphenylsulfonyl chloride (0.8 mmol, 190 mg), the reaction mixture was stirred at room temperature for 3 hours. Water was then added to the reaction mixture, which was extracted with ethyl acetate. The collected extracts were washed with saturated sodium chloride solution and dried over magnesium sulfate. Purification by chromatography (silica gel, 4% MeOH in dichloromethane) resulted in 352 mg (64%) of the product.

Mass spectrum: m/z=722 [M+H]

Rotation: α^{20° C.}_{D}=−92 (c=0.1 in chloroform)

D) (2S,4R)-1-[5-Cyano-1-(2,4-dimethoxybenzenesulfonyl)-3-(2-methoxyphenyl)-2-oxo-2,3-dihydro-1H-indol-3-yl]-4-hydroxypyrrolidine-2-dimethylcarboxamide α(20° C., c=1 mg/ml, CHCl₃, I=1 dm):-186

A solution of example 1C (0.1 mmol, 72 mg), zinc cyanide (0.07 mmol, 8 mg) and palladium(0) tetrakistriphenylphosphines (15 mg) was heated at 75° C. for 18 hours. The reaction mixture was partitioned between water and ethyl acetate. The organic phase was washed with water and saturated sodium chloride solution and dried over magnesium sulfate. Purification by chromatography (silica gel, gradient: 3% to 7% MeOH in dichloromethane) resulted in 39 mg (63%) of example 1.

Mass spectrum: m/z=621 [M+H]

¹H NMR (400 MHz, DMSO-d₆) δ 8.00 (d, 1H), 7.93 (m, 1H), 7.85 (m, 2H), 7.40 (s, 1H), 7.30 (t, 1H), 6.97 (t, 1H), 6.90 (d, 1H), 6.75 (d, 1H), 6.70 (s, 1H), 4.90 (br s, 1H), 4.57 (m, 1H), 4.35 (m, 1H), 3.85 (s, 3H), 3.70 (s, 3H), 2.90 (m), 2.55 (m), 2.35 (m), 1.60 (m, 1H).

### (2S,4R)-4-Hydroxypyrrolidine-2-dimethylcarboxamide hydrochloride (method according to WO 01/55130)

A) (Benzotriazol-1-yloxy)tris(dimethylamino)phosphonium hexafluorophosphate (BOP) (172 g, 0.389 mol) was added in portions to a solution of (2S,4R)-1-(tert-butoxycarbonyl)-4-hydroxypyrrolidine-2-carboxylic acid (90 g, 0.389 mol) in dichloromethane (450 ml) and N-ethyldiisopropylamine (DIPEA) (68 ml, 0.523 mol) at 0° C., and the mixture was stirred at 0° C. for 1 hour. Subsequently, a 2 M solution of dimethylamine in THF (800 ml, 1.6 mol) was added dropwise at 0° C., and the mixture was stirred at room temperature overnight. The reaction mixture was stirred into ice-water and the mixture was extracted several times with dichloromethane. The collected organic phase was washed with saturated sodium chloride solution, dried over magnesium sulfate and concentrated under reduced pressure.
B) The product from step A) was mixed with 500 ml of 5-6 M HCl in isopropanol and stirred at room temperature for 4 hours. After cooling to 0° C., the precipitate was filtered off with suction, washed with isopropanol and diethyl ether and dried. 37 g of the desired product were obtained.

### Example 2

### (±)-(2S)-1-[5-Cyano-1-(4-methoxybenzenesulfonyl)-3-(2-methoxyphenyl)-2-oxo-2,3-dihydro-1H-indol-3-yl]pyrrolidine-2-dimethylcarboxamide

Reaction in analogy to example 1-D of (±)-(2S)-1-[5-cyano-3-(2-methoxyphenyl)-2-oxo-2,3-dihydro-1H-indol-3-yl]pyrrolidine-2-dimethylcarboxamide (1215-145, 0.74 mmol, 0.30 g) with 4-methoxybenzenesulfonyl chloride (0.82 mmol, 0.17 g) resulted in 0.32 g of the title compound.

ESI-MS: [M+H⁺]=575.2;

### (±)-(2S)-1-[5-Cyano-3-(2-methoxyphenyl)-2-oxo-2,3-dihydro-1H-indol-3-yl]-pyrrolidine-2-dimethylcarboxamide

4 g of 3-chloro-3-(2-methoxyphenyl)-2-oxo-2,3-dihydro-1H-indole-5-carbonitrile and 5.7 ml of DIPEA were dissolved in 100 ml of CH₂Cl₂. Addition of 1.9 g of (2S)-proline dimethylamide was followed by stirring the reaction solution at room temperature for 16 h. The solution was then diluted with water. The aqueous phase was then extracted 2× with CH₂Cl₂. The combined organic phases were washed with aqueous NaHCO₃ and with water, dried and concentrated in vacuo. The residue obtained in this way was purified by chromatography (eluent: CH₂Cl₂/MeOH=20/1) 1.55 g of the product were obtained.

### 3-Chloro-3-(2-methoxyphenyl)-2-oxo-2,3-dihydro-1H-indole-5-carbonitrile

10.6 g (37.8 mmol) of 3-hydroxy-3-(2-methoxyphenyl)-2-oxo-2,3-dihydro-1H-indole-5-carbonitrile and 6.2 ml (75.6 mmol) of pyridine were put into 80 ml of CH₂Cl₂ and, at 0° C., 4.1 ml (56.7 mmol) of SOCl₂ were cautiously added. The mixture was stirred at 0° C. for 1 h. The reaction mixture was poured into ice-water, and the aqueous phase was extracted with CH₂Cl₂. The organic phase was then washed with water, dried and concentrated in vacuo. The residue was treated with a little CH₂Cl₂, and the resulting crystals were isolated. 5.7 g of the product were obtained.

### 3-Hydroxy-3-(2-methoxyphenyl)-2-oxo-2,3-dihydro-1H-indole-5-carbonitrile

20 g (52.5 mmol) of 3-hydroxy-5-iodo-3-(2-methoxyphenyl)-1,3-dihydroindol-2-one, 6.2 g of Zn(CN)₂, and 3 g (2.6 mmol) of [[C₆H₅)₃P]4 Pd were put into 200 ml of DMF and heated at 75° C. for 1 h. The mixture was then diluted with water, and the aqueous phase was extracted with ethyl acetate. The organic phase was then washed 3× with water, dried and concentrated in vacuo. The resulting residue was treated with a little ethyl acetate, and the resulting solid was isolated. 11 g of the product were obtained.

### 3-Hydroxy-5-iodo-3-(2-methoxyphenyl)-1,3-dihydroindol-2-one

25 g (0.09 mol) of 5-iodoisatin were introduced in portions into 360 ml (0.36 mol) of 1 M 2-methoxyphenylmagnesium bromide solution in THF (Aldrich) at 15° C. The mixture was then stirred for 30 minutes. The reaction solution was subsequently stirred into ice-cooled 10% strength NH₄Cl solution. The aqueous phase was extracted with ethyl acetate, separated off, washed several times with water, dried and concentrated in vacuo. The resulting residue was treated with a little ethyl acetate and then the resulting solid was isolated. 20 g of the product were obtained.

### Example 3

### (+)-(2S)-1-[5-Cyano-1-(2,4-dimethoxybenzenesulfonyl)-3-(2-methoxyphenyl)-2-oxo-2,3-dihydro-1H-indol-3-yl]pyrrolidine-2-dimethylcarboxamide

Reaction in analogy to example 1-D of (±)-(2S)-1-[5-cyano-3-(2-methoxyphenyl)-2-oxo-2,3-dihydro-1H-indol-3-yl]pyrrolidine-2-dimethylcarboxamide (0.49 mmol, 0.20 g) with 2,4-dimethoxybenzenesulfonyl chloride (0.64 mmol, 0.15 g) resulted in 49.0 mg of the title compound.

α(20° C., c=1 mg/ml, CHCl₃, I=1 dm): +82°;

ESI-MS: [M+H⁺]=605.3;

### Example 4

### (−)-(2S)-1-[5-Cyano-1-(2,4-dimethoxybenzenesulfonyl)-3-(2-methoxyphenyl)-2-oxo-2,3-dihydro-1H-indol-3-yl]pyrrolidine-2-dimethylcarboxamide

Reaction in analogy to example 1-D of (±)-(2S)-1-[5-cyano-3-(2-methoxyphenyl)-2-oxo-2,3-dihydro-1H-indol-3-yl]pyrrolidine-2-dimethylcarboxamide (0.49 mmol, 0.20 g) with 2,4-dimethoxybenzenesulfonyl chloride (0.64 mmol, 0.15 g) resulted in 43.0 mg of the title compound.

α(20° C., c=1 mg/ml, CHCl₃, I=1 dm): −150°;

ESI-MS: [M+H⁺]=605.3;

### Example 5

### (±)-(2S)-1-[5-Cyano-1-(2,4-dimethoxybenzenesulfonyl)-3-(2-methoxyphenyl)-2-oxo-2,3-dihydro-1H-indol-3-yl]piperidine-2-dimethylcarboxamide

Reaction in analogy to example 1-D of (±)-(2S)-1-[5-cyano-3-(2-methoxyphenyl)-2-oxo-2,3-dihydro-1H-indol-3-yl]piperidine-2-dimethylcarboxamide (0.76 mmol, 0.32 g) with 2,4-dimethoxybenzenesulfonyl chloride (0.80 mmol, 0.19 g) resulted in 0.30 g of the title compound.

ESI-MS: [M+H⁺]=619.2;

(±)-(2S)-1-[5-Cyano-3-(2-methoxyphenyl)-2-oxo-2,3-dihydro-1H-indol-3-yl]piperidine-2-dimethylcarboxamide

Reaction in analogy to example 2 of 3-chloro-3-(2-methoxyphenyl)-2-oxo-2,3-dihydro-1H-indole-5-carbonitrile (1.96 mmol, 0.59 g) with (2S)-piperidine-2-dimethylcarboxamide hydrochloride (WO 01/74775, 2.06 mmol, 0.40 g) resulted in 0.85 g of the title compound.

ESI-MS: [M+H⁺]=419.15;

### (2S)-Piperidine-2-dimethylcarboxamide hydrochloride (WO 01/74775)

20 g (87.2 mmol) of (S)-1-(tert-butoxycarbonyl)-2-piperidinecarboxylic acid and 13 g (96 mmol) of 1-hydroxybenzotriazole (HOBT) were dissolved in 300 ml of DMF. Addition of 150 ml (305 mmol) of a solution of 2 M dimethylamine in THF was followed by cooling the solution to 10° C. Then 18.4 (96 mmol) of N-(3-dimethylaminopropyl)-N′-ethylcarbodiimide (EDAC or EDCI) were added in portions. The reaction solution was stirred at room temperature for 16 hours. The reaction solution was highly concentrated in vacuo, and the resulting residue was partitioned between ethyl acetate and water. The organic phase was washed 2× with water, 3× with 5% strength K₂CO₃ solution and again with water, dried and concentrated in vacuo. The resulting residue was dissolved in ether, and 100 ml of 5-6 M isopropanolic HCl were added. The reaction mixture was cautiously heated at 30° C. for 1 hour and then concentrated in vacuo. 14.6 g of the product were obtained.

### Example 6

### (+)-(2S)-2-{[5-Cyano-1-(2,4-dimethoxybenzenesulfonyl)-3-(2-methoxyphenyl)-2-oxo-2,3-dihydro-1H-indol-3-yl]methylamino}-N,N-dimethylpropionamide

Reaction in analogy to example 1-D of (±)-(2S)-2-{[5-cyano-3-(2-methoxyphenyl)-2-oxo-2,3-dihydro-1H-indol-3-yl]methylamino}-N,N-dimethylpropionamide (0.64 mmol, 0.25 g) with 2,4-dimethoxybenzenesulfonyl chloride (0.67 mmol, 0.16 g) resulted in 0.18 g of the title compound.

ESI-MS: [M+H⁺]=593.2;

α(20° C., c=1 mg/ml, CHCl₃, I=1 dm): +164

### (±)-(2S)-2-{[5-Cyano-3-(2-methoxyphenyl)-2-oxo-2,3-dihydro-1H-indol-3-yl]methyl-amino}-N,N-dimethylpropionamide

Reaction in analogy to example 2-1215/145 of 3-chloro-3-(2-methoxyphenyl)-2-oxo-2,3-dihydro-1H-indole-5-carbonitrile (6.03 mmol, 1.80 g) with (2S)—N,N-dimethyl-2-methylaminopropionamide hydrochloride (6.03 mmol, 1.00 g) resulted in 2.20 g of the title compound.

ESI-MS: [M+H⁺]=393.15;

(S)—N,N-Dimethyl-2-methylaminopropionamide hydrochloride

1-Hydroxy-1H-benzotriazole (10.8 mmol, 1.46 g) and N-(3-dimethylaminopropyl)-N′-ethylcarbodiimide (EDCI or EDAC) (10.8 mmol, 2.08 g) were added to a solution of (S)-Boc-N-Me-Ala-OH (9.8 mmol, 2.00 g, Bachem) in DMF (10 ml). After stirring at room temperature for 10 min, a 2 M solution of dimethylamine in THF (11.8 mmol, 5.9 ml) was added dropwise. The reaction mixture was stirred at room temperature for 18 h. Addition of water was followed by extraction of the mixture with ethyl acetate several times. The combined organic phase was washed with 1N hydrochloric acid, sodium bicarbonate solution and sodium chloride solution. After drying over magnesium sulfate, the solvent was stripped off under reduced pressure. Yield: 1.86 g of colorless oil (82%). The Boc-protected intermediate was dissolved in methanol (19 ml) and treated with a 4N solution of HCl in dioxane (32 mmol, 8 ml). After stirring at room temperature for 18 h, the solvent was removed under reduced pressure and the product was dried in vacuo. Yield: 1.41 g of a white solid (quantitative).

### Example 7

### (−)-(2S,4R)-1-[5-Cyano-1-(4-methoxybenzenesulfonyl)-3-(2-methoxyphenyl)-2-oxo-2,3-dihydro-1H-indol-3-yl]-4-hydroxypyrrolidine-2-dimethylcarboxamide

Reaction in analogy to example 1-D of (±)-(2S,4R)-1-[5-cyano-3-(2-methoxyphenyl)-2-oxo-2,3-dihydro-1H-indol-3-yl]-4-hydroxypyrrolidine-2-dimethylcarboxamide (0.31 mmol, 0.13 g) with 4-methoxybenzenesulfonyl chloride (0.32 mmol, 0.07 g) resulted in 0.14 g of the title compound.

ESI-MS: [M+H⁺]=591.2;

α(20° C., c=1 mg/ml, CHCl₃, I=1 dm): −130

### Example 8

### (−)-(2S,4R)-1-[5-Cyano-1-(2,4-dichlorobenzenesulfonyl)-3-(2-methoxyphenyl)-2-oxo-2,3-dihydro-1H-indol-3-yl]-4-hydroxypyrrolidine-2-dimethylcarboxamide

Reaction in analogy to example 1-D of (±)-(2S,4R)-1-[5-cyano-3-(2-methoxyphenyl)-2-oxo-2,3-dihydro-1H-indol-3-yl]-4-hydroxypyrrolidine-2-dimethylcarboxamide (0.31 mmol, 0.13 g) with 2,4-dichlorobenzenesulfonyl chloride (0.32 mmol, 0.08 g) resulted in 0.15 g of the title compound.

ESI-MS: 631.0, [M+H⁺]=630.0, 629.0;

### Example 9

### (−)-(2S)-2-{[5-Cyano-1-(2,4-dimethoxybenzenesulfonyl)-3-(2-methoxyphenyl)-2-oxo-2,3-dihydro-1H-indol-3-yl]methylamino}-N,N-dimethylpropionamide

Reaction in analogy to example 1-D of (±)-(2S)-2-{[5-cyano-3-(2-methoxyphenyl)-2-oxo-2,3-dihydro-1H-indol-3-yl]methylamino}-N,N-dimethylpropionamide (0.64 mmol, 0.25 g) with 2,4-dimethoxybenzenesulfonyl chloride (0.67 mmol, 0.16 g) resulted in 0.06 g of the title compound.

ESI-MS: [M+H⁺]=593.2;

### Example 10

### (±)-(2S)-1-[5-Cyano-1-(4-cyanobenzenesulfonyl)-3-(2-methoxyphenyl)-2-oxo-2,3-dihydro-1H-indol-3-yl]pyrrolidine-2-dimethylcarboxamide

Reaction in analogy to example 1-D of (±)-(2S)-1-[5-cyano-3-(2-methoxyphenyl)-2-oxo-2,3-dihydro-1H-indol-3-yl]pyrrolidine-2-dimethylcarboxamide (0.49 mmol, 0.20 g) with 4-cyanobenzenesulfonyl chloride (0.52 mmol, 0.10 g) resulted in 0.25 g of the title compound.

ESI-MS: [M+H⁺]=570.2;

### Example 11

### (±)-(2S)-1-[5-Cyano-1-(4-ethylbenzenesulfonyl)-3-(2-methoxyphenyl)-2-oxo-2,3-dihydro-1H-indol-3-yl]pyrrolidine-2-dimethylcarboxamide

Reaction in analogy to example 1-D of (±)-(2S)-1-[5-cyano-3-(2-methoxyphenyl)-2-oxo-2,3-dihydro-1H-indol-3-yl]pyrrolidine-2-dimethylcarboxamide (0.49 mmol, 0.20 g) with 4-ethylbenzenesulfonyl chloride (0.52 mmol, 0.11 g) resulted in 0.19 g of the title compound.

ESI-MS: [M+H⁺]=573.2;

### Example 12

### (±)-(2S)-2-{[1-(4-Chlorobenzenesulfonyl)-5-cyano-3-(2-methoxyphenyl)-2-oxo-2,3-dihydro-1H-indol-3-yl]methylamino}-N,N-dimethylpropionamide

Reaction in analogy to example 1-D of (±)-(2S)-2-{[5-cyano-3-(2-methoxyphenyl)-2-oxo-2,3-dihydro-1H-indol-3-yl]methylamino}-N,N-dimethylpropionamide (0.51 mmol, 0.20 g) with 4-chlorobenzenesulfonyl chloride (0.54 mmol, 0.11 g) resulted in 0.29 g of the title compound.

ESI-MS: [M+H⁺]=570.1, 569.0, 567.1;

### Example 13

### (±)-(2S)-2-{[5-Cyano-3-(2-methoxyphenyl)-2-oxo-1-(4-trifluoromethoxybenzene-sulfonyl)-2,3-dihydro-1H-indol-3-yl]methylamino}-N,N-dimethylpropionamide

Reaction in analogy to example 1-D of (±)-(2S)-2-{[5-cyano-3-(2-methoxyphenyl)-2-oxo-2,3-dihydro-1H-indol-3-yl]methylamino}-N,N-dimethylpropionamide (0.51 mmol, 0.20 g) with 4-trifluoromethoxybenzenesulfonyl chloride (0.54 mmol, 0.14 g) resulted in 0.30 g of the title compound.

ESI-MS: [M+H⁺]=617.2;

### Example 14

### (±)-(2S)-2-{[5-Cyano-1-(4-isopropylbenzenesulfonyl)-3-(2-methoxyphenyl)-2-oxo-2,3-dihydro-1H-indol-3-yl]methylamino}-N,N-dimethylpropionamide

Reaction in analogy to example 1-D of (±)-(2S)-2-{[5-cyano-3-(2-methoxyphenyl)-2-oxo-2,3-dihydro-1H-indol-3-yl]methylamino}-N,N-dimethylpropionamide (0.51 mmol, 0.20 g) with 4-isopropylbenzenesulfonyl chloride (0.54 mmol, 0.12 g) resulted in 0.29 g of the title compound.

ESI-MS: [M+H⁺]=575.2;

### Example 15

(±)-(2S)-2-{[5-Cyano-1-(4-methoxybenzenesulfonyl)-3-(2-methoxyphenyl)-2-oxo-2,3-dihydro-1H-indol-3-yl]methylamino}-N,N-dimethylpropionamide

Reaction in analogy to example 1-D of (±)-(2S)-2-{[5-cyano-3-(2-methoxyphenyl)-2-oxo-2,3-dihydro-1H-indol-3-yl]methylamino}-N,N-dimethylpropionamide (0.64 mmol, 0.25 g) with 4-methoxybenzenesulfonyl chloride (0.67 mmol, 0.14 g) resulted in 0.30 g of the title compound.

ESI-MS: [M+H⁺]=563.2;

### Example 16

### (+)-(2S,4R)-1-[5-Cyano-1-(2,4-dimethoxybenzenesulfonyl)-3-(2-methoxyphenyl)-2-oxo-2,3-dihydro-1H-indol-3-yl]-4-hydroxypyrrolidine-2-dimethylcarboxamide

Reaction in analogy to example 1-D of (±)-(2S,4R)-1-[5-cyano-3-(2-methoxyphenyl)-2-oxo-2,3-dihydro-1H-indol-3-yl]-4-hydroxypyrrolidine-2-dimethylcarboxamide

(0.59 mmol, 0.25 g) with 2,4-dimethoxybenzenesulfonyl chloride (0.65 mmol, 0.15 g) resulted in 48.0 mg of the title compound.

ESI-MS: [M+H⁺]=621.2;

### Examples 17a/b

### Purification of the Diastereomeric Compounds of Example 2: (±)-(2S)-1-[5-cyano-1-(4-methoxybenzenesulfonyl)-3-(2-methoxyphenyl)-2-oxo-2,3-dihydro-1H-indol-3-yl]pyrrolidine-2-dimethylcarboxamide

The diastereomers of (±)-(2S)-1-[5-cyano-1-(4-methoxybenzenesulfonyl)-3-(2-methoxyphenyl)-2-oxo-2,3-dihydro-1H-indol-3-yl]pyrrolidine-2-dimethylcarboxamide from example 2 were separated by HPLC prep. (XterraPrepC18 (Waters, 250×30 mm, 10 μm), eluent H₂O/CH₃CN 0.1% AcOH (v/v)).

(−)-(2S)-1-[5-Cyano-1-(4-methoxybenzenesulfonyl)-3-(2-methoxyphenyl)-2-oxo-2,3-dihydro-1H-indol-3-yl]pyrrolidine-2-dimethylcarboxamide (example 17a)

ESI-MS: [M+H⁺]=575.15;

α(20° C., c=1 mg/ml, CHCl₃, I=1 dm): −181°;

### (+)-(2S)-1-[5-Cyano-1-(4-methoxybenzenesulfonyl)-3-(2-methoxyphenyl)-2-oxo-2,3-dihydro-1H-indol-3-yl]pyrrolidine-2-dimethylcarboxamide (example 17b)

ESI-MS: [M+H⁺]=575.15;

α(20° C., c=1 mg/ml, CHCl₃, I=1 dm): +89°;

### Example 18a/b

### Purification of the Diastereomeric Compounds of Example 5: (±)-(2S)-1-[5-cyano-1-(2,4-dimethoxybenzenesulfonyl)-3-(2-methoxyphenyl)-2-oxo-2,3-dihydro-1H-indol-3-yl]piperidine-2-dimethylcarboxamide

The diastereomers of (±)-(2S)-1-[5-cyano-1-(2,4-dimethoxybenzenesulfonyl)-3-(2-methoxyphenyl)-2-oxo-2,3-dihydro-1H-indol-3-yl]piperidine-2-dimethylcarboxamide from example 5 were separated by chiral HPLC prep. (Chiralcel OD (Daicel, 250×4.6 mm), eluent hexane/EtOH/Et₃N 85/15/0.1 (v/v)). (−)-(2S)-1-[5-Cyano-1-(2,4-dimethoxybenzenesulfonyl)-3-(2-methoxyphenyl)-2-oxo-2,3-dihydro-1H-indol-3-yl]piperidine-2-dimethylcarboxamide (example 18a)

ESI-MS: [M+H⁺]=619.15;

### (+)-(2S)-1-[5-Cyano-1-(2,4-dimethoxybenzenesulfonyl)-3-(2-methoxyphenyl)-2-oxo-2,3-dihydro-1H-indol-3-yl]piperidine-2-dimethylcarboxamide (example 18b)

ESI-MS: [M+H⁺]=619.15;

### Example 19

### Purification of One of the Diastereomeric Compounds of Example 7

The purification took place in analogy to example 18a/b.

(−)-(2S,4R)-1-[5-Cyano-1-(4-methoxybenzenesulfonyl)-3-(2-methoxyphenyl)-2-oxo-2,3-dihydro-1H-indol-3-yl]-4-hydroxypyrrolidine-2-dimethylcarboxamide a (20° C., c=1 mg/ml, CHCl₃, I=1 dm): −130°;

### Example 20a/b

### Purification of the diastereomeric compounds of example 11

### (±)-(2S)-1-[5-Cyano-1-(4-ethylbenzenesulfonyl)-3-(2-methoxyphenyl)-2-oxo-2,3-dihydro-1H-indol-3-yl]pyrrolidine-2-dimethylcarboxamide)

The diastereomers of (±)-(2S)-1-[5-cyano-1-(4-ethylbenzenesulfonyl)-3-(2-methoxyphenyl)-2-oxo-2,3-dihydro-1H-indol-3-yl]pyrrolidine-2-dimethylcarboxamide from example 11 were separated by chromatography (RP cartridge (Macherey Nagel, Chromabond C18), eluent H₂O/CH₃CN(20-45%)/AcOH (0.1%) (v/v)).

(−)-(2S)-1-[5-Cyano-1-(4-ethylbenzenesulfonyl)-3-(2-methoxyphenyl)-2-oxo-2,3-dihydro-1H-indol-3-yl]pyrrolidine-2-dimethylcarboxamide (example 20a)

ESI-MS: [M+H⁺]=573.15;

### (+)-(2S)-1-[5-Cyano-1-(4-ethylbenzenesulfonyl)-3-(2-methoxyphenyl)-2-oxo-2,3-dihydro-1H-indol-3-yl]pyrrolidine-2-dimethylcarboxamide (example 20b)

ESI-MS: [M+H⁺]=573.15;

Further examples of compounds of the invention and of the compounds of the general formula (I)

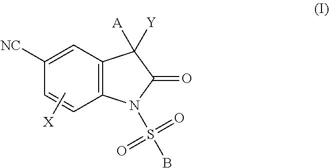

in which the variables A, B, X and Y are each independently of one another selected from the group consisting of
X=hydrogen (H), methoxy (OCH3), methyl (CH3) and chlorine (Cl);
A=2-methoxyphenyl (2-OCH₃-Ph) and 2-chlorophenyl (2-Cl-Ph);
B=2,4-dimethoxyphenyl, 4-methoxyphenyl (4-OCH₃-Ph), 4-chlorophenyl (4-Cl-Ph), 4-fluorophenyl (4-F-Ph), 4-cyanophenyl (4-CN-Ph), 4-trifluoromethoxyphenyl (4-OCF₃-Ph), 4-isopropylphenyl (4-isopropyl-Ph), 2,4-difluorophenyl (2,4-difluoro-Ph), 2-methoxy-4-methylphenyl (2-methoxy-4-methyl-Ph), 4-methylphenyl (4-methyl-Ph), 2-fluorophenyl (2-F-Ph), 2,4-dichlorphenyl (2,4-dichloro-Ph), 4-ethylphenyl (4-Et-Ph), 4-acetylphenyl (4-Ac-Ph), 3,4-dimethoxyphenyl (3,4-dimethoxy-Ph) and 3-chlorophenyl (3-Cl-Ph);
Y═Y1, Y2, Y3, Y4, Y5, Y6, Y7, Y8, Y9, Y10, Y11 and Y12, where Y1-Y12 are intended to have the meanings mentioned below

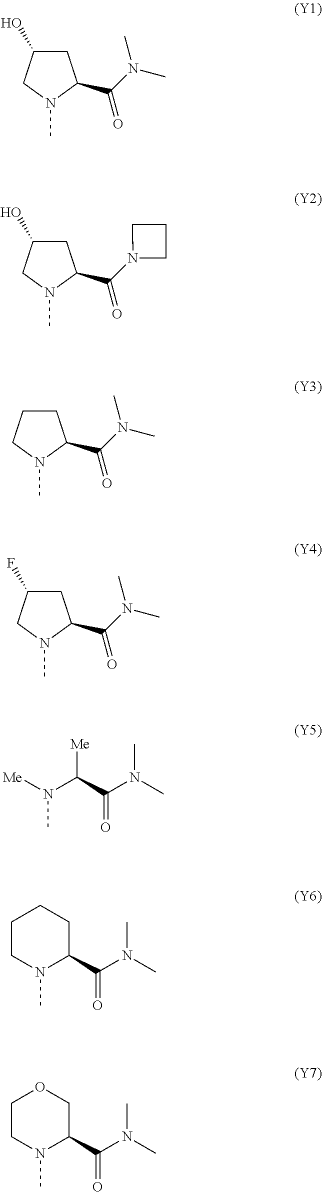

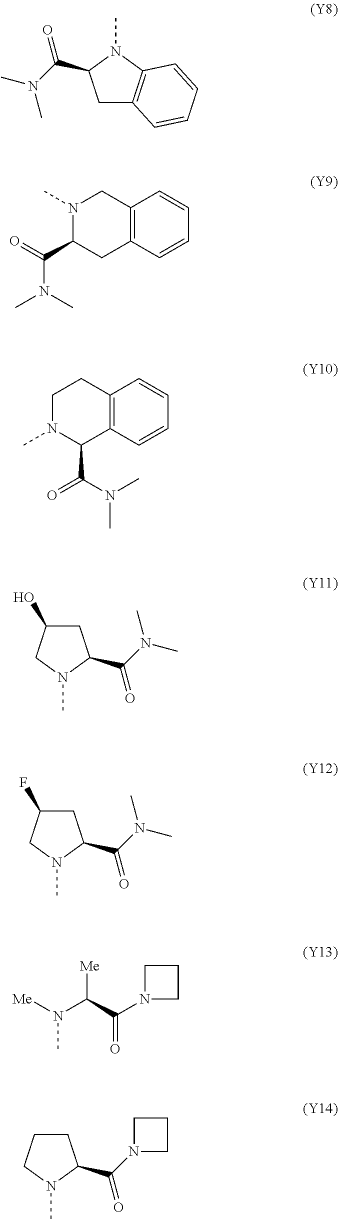

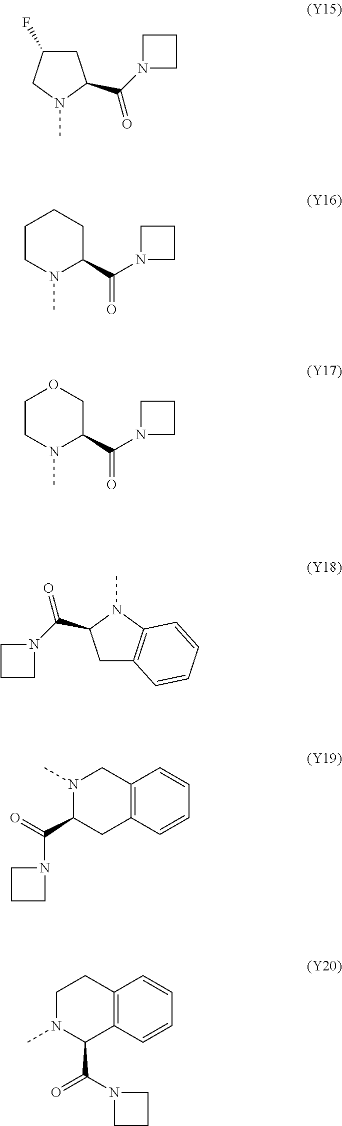

Examples of the abovementioned compounds of the invention are listed in the form of compounds of the above general formula (I) in table 1 below, where the radicals A, B, X and Y are each intended together to have the meanings indicated in one line of table 1.

TABLE 1

| Example | A | B | X | Y |
|---|---|---|---|---|
| 21 | 2-OCH₃—Ph | 4-Cl—Ph | H | Y1 |
| 22 | 2-OCH₃—Ph | 4-F—Ph | H | Y1 |
| 23 | 2-OCH₃—Ph | 4-CN—Ph | H | Y1 |
| 44 | 2-OCH₃—Ph | 4-OCH3—Ph | H | Y1 |
| 25 | 2-Cl—Ph | 4-Cl—Ph | H | Y1 |
| 26 | 2-Cl—Ph | 4-F—Ph | H | Y1 |
| 27 | 2-Cl—Ph | 4-CN—Ph | H | Y1 |
| 28 | 2-Cl—Ph | 4-OCH3—Ph | H | Y1 |
| 29 | 2-OCH3—Ph | 4-Cl—Ph | H | Y2 |
| 30 | 2-OCH3—Ph | 4-F—Ph | H | Y2 |
| 31 | 2-OCH3—Ph | 4-CN—Ph | H | Y2 |
| 32 | 2-OCH3—Ph | 4-OCH3—Ph | H | Y2 |
| 33 | 2-Cl—Ph | 4-Cl—Ph | H | Y2 |
| 34 | 2-Cl—Ph | 4-F—Ph | H | Y2 |
| 35 | 2-Cl—Ph | 4-CN—Ph | H | Y2 |
| 36 | 2-Cl—Ph | 4-OCH3—Ph | H | Y2 |
| 37 | 2-OCH3—Ph | 4-Cl—Ph | H | Y3 |
| 38 | 2-OCH3—Ph | 4-F—Ph | H | Y3 |
| 39 | 2-OCH3—Ph | 4-CN—Ph | H | Y3 |
| 40 | 2-OCH3—Ph | 4-OCH3—Ph | H | Y3 |
| 41 | 2-Cl—Ph | 4-Cl—Ph | H | Y3 |
| 42 | 2-Cl—Ph | 4-F—Ph | H | Y3 |
| 43 | 2-Cl—Ph | 4-CN—Ph | H | Y3 |
| 44 | 2-Cl—Ph | 4-OCH3—Ph | H | Y3 |
| 45 | 2-OCH3—Ph | 4-Cl—Ph | H | Y14 |
| 46 | 2-OCH3—Ph | 4-F—Ph | H | Y14 |
| 47 | 2-OCH3—Ph | 4-CN—Ph | H | Y14 |
| 48 | 2-OCH3—Ph | 4-OCH3—Ph | H | Y14 |
| 49 | 2-Cl—Ph | 4-Cl—Ph | H | Y14 |
| 50 | 2-Cl—Ph | 4-F—Ph | H | Y14 |
| 51 | 2-Cl—Ph | 4-CN—Ph | H | Y14 |
| 52 | 2-Cl—Ph | 4-OCH3—Ph | H | Y14 |
| 53 | 2-OCH3—Ph | 4-Cl—Ph | H | Y4 |
| 54 | 2-OCH3—Ph | 4-F—Ph | H | Y4 |
| 55 | 2-OCH3—Ph | 4-CN—Ph | H | Y4 |
| 56 | 2-OCH3—Ph | 4-OCH3—Ph | H | Y4 |
| 57 | 2-Cl—Ph | 4-Cl—Ph | H | Y4 |
| 58 | 2-Cl—Ph | 4-F—Ph | H | Y4 |
| 59 | 2-Cl—Ph | 4-CN—Ph | H | Y4 |
| 50 | 2-Cl—Ph | 4-OCH3—Ph | H | Y4 |
| 61 | 2-OCH3—Ph | 4-Cl—Ph | H | Y15 |
| 62 | 2-OCH3—Ph | 4-F—Ph | H | Y15 |
| 63 | 2-OCH3—Ph | 4-CN—Ph | H | Y15 |
| 64 | 2-OCH3—Ph | 4-OCH3—Ph | H | Y15 |
| 65 | 2-Cl—Ph | 4-Cl—Ph | H | Y15 |
| 66 | 2-Cl—Ph | 4-F—Ph | H | Y15 |
| 67 | 2-Cl—Ph | 4-CN—Ph | H | Y15 |
| 68 | 2-Cl—Ph | 4-OCH3—Ph | H | Y15 |
| 69 | 2-OCH3—Ph | 4-Cl—Ph | H | Y5 |
| 70 | 2-OCH3—Ph | 4-F—Ph | H | Y5 |
| 71 | 2-OCH3—Ph | 4-CN—Ph | H | Y5 |
| 72 | 2-OCH3—Ph | 4-OCH3—Ph | H | Y5 |
| 73 | 2-Cl—Ph | 4-Cl—Ph | H | Y5 |
| 74 | 2-Cl—Ph | 4-F—Ph | H | Y5 |
| 75 | 2-Cl—Ph | 4-CN—Ph | H | Y5 |
| 76 | 2-Cl—Ph | 4-OCH3—Ph | H | Y5 |
| 77 | 2-OCH3—Ph | 4-Cl—Ph | H | Y13 |
| 78 | 2-OCH3—Ph | 4-F—Ph | H | Y13 |
| 79 | 2-OCH3—Ph | 4-CN—Ph | H | Y13 |
| 80 | 2-OCH3—Ph | 4-OCH3—Ph | H | Y13 |
| 81 | 2-Cl—Ph | 4-Cl—Ph | H | Y13 |
| 82 | 2-Cl—Ph | 4-F—Ph | H | Y13 |
| 83 | 2-Cl—Ph | 4-CN—Ph | H | Y13 |
| 84 | 2-Cl—Ph | 4-OCH3—Ph | H | Y13 |
| 85 | 2-OCH3—Ph | 4-Cl—Ph | H | Y6 |
| 86 | 2-OCH3—Ph | 4-F—Ph | H | Y6 |
| 87 | 2-OCH3—Ph | 4-CN—Ph | H | Y6 |
| 88 | 2-OCH3—Ph | 4-OCH3—Ph | H | Y6 |
| 89 | 2-Cl—Ph | 4-Cl—Ph | H | Y6 |
| 90 | 2-Cl—Ph | 4-F—Ph | H | Y6 |
| 91 | 2-Cl—Ph | 4-CN—Ph | H | Y6 |
| 92 | 2-Cl—Ph | 4-OCH3—Ph | H | Y6 |
| 93 | 2-OCH3—Ph | 4-Cl—Ph | H | Y16 |
| 94 | 2-OCH3—Ph | 4-F—Ph | H | Y16 |
| 95 | 2-OCH3—Ph | 4-CN—Ph | H | Y16 |
| 96 | 2-OCH3—Ph | 4-OCH3—Ph | H | Y16 |
| 97 | 2-Cl—Ph | 4-Cl—Ph | H | Y16 |
| 98 | 2-Cl—Ph | 4-F—Ph | H | Y16 |
| 99 | 2-Cl—Ph | 4-CN—Ph | H | Y16 |
| 100 | 2-Cl—Ph | 4-OCH3—Ph | H | Y16 |
| 101 | 2-OCH3—Ph | 4-Cl—Ph | H | Y7 |
| 102 | 2-OCH3—Ph | 4-F—Ph | H | Y7 |
| 103 | 2-OCH3—Ph | 4-CN—Ph | H | Y7 |
| 104 | 2-OCH3—Ph | 4-OCH3—Ph | H | Y7 |
| 105 | 2-Cl—Ph | 4-Cl—Ph | H | Y7 |
| 106 | 2-Cl—Ph | 4-F—Ph | H | Y7 |
| 107 | 2-Cl—Ph | 4-CN—Ph | H | Y7 |
| 108 | 2-Cl—Ph | 4-OCH3—Ph | H | Y7 |
| 109 | 2-OCH3—Ph | 4-Cl—Ph | H | Y17 |
| 110 | 2-OCH3—Ph | 4-F—Ph | H | Y17 |
| 111 | 2-OCH3—Ph | 4-CN—Ph | H | Y17 |
| 112 | 2-OCH3—Ph | 4-OCH3—Ph | H | Y17 |
| 113 | 2-Cl—Ph | 4-Cl—Ph | H | Y17 |
| 114 | 2-Cl—Ph | 4-F—Ph | H | Y17 |
| 115 | 2-Cl—Ph | 4-CN—Ph | H | Y17 |
| 116 | 2-Cl—Ph | 4-OCH3—Ph | H | Y17 |
| 117 | 2-OCH3—Ph | 4-Cl—Ph | H | Y11 |
| 118 | 2-OCH3—Ph | 4-F—Ph | H | Y11 |
| 119 | 2-OCH3—Ph | 4-CN—Ph | H | Y11 |
| 120 | 2-OCH3—Ph | 4-OCH3—Ph | H | Y11 |
| 121 | 2-Cl—Ph | 4-Cl—Ph | H | Y11 |
| 122 | 2-Cl—Ph | 4-F—Ph | H | Y11 |
| 123 | 2-Cl—Ph | 4-CN—Ph | H | Y11 |
| 124 | 2-Cl—Ph | 4-OCH3—Ph | H | Y11 |
| 125 | 2-OCH3—Ph | 4-Cl—Ph | H | Y12 |
| 126 | 2-OCH3—Ph | 4-F—Ph | H | Y12 |
| 127 | 2-OCH3—Ph | 4-CN—Ph | H | Y12 |
| 128 | 2-OCH3—Ph | 4-OCH3—Ph | H | Y12 |
| 129 | 2-Cl—Ph | 4-Cl—Ph | H | Y12 |
| 130 | 2-Cl—Ph | 4-F—Ph | H | Y12 |
| 131 | 2-Cl—Ph | 4-CN—Ph | H | Y12 |
| 132 | 2-Cl—Ph | 4-OCH3—Ph | H | Y12 |
| 133 | 2-OCH3—Ph | 4-Cl—Ph | CH₃ | Y1 |
| 134 | 2-OCH3—Ph | 4-F—Ph | CH₃ | Y1 |
| 135 | 2-OCH3—Ph | 4-CN—Ph | CH₃ | Y1 |
| 136 | 2-OCH3—Ph | 4-OCH3—Ph | CH₃ | Y1 |
| 137 | 2-Cl—Ph | 4-Cl—Ph | CH₃ | Y1 |
| 138 | 2-Cl—Ph | 4-F—Ph | CH₃ | Y1 |
| 139 | 2-Cl—Ph | 4-CN—Ph | CH₃ | Y1 |
| 140 | 2-Cl—Ph | 4-OCH3—Ph | CH₃ | Y1 |
| 141 | 2-OCH3—Ph | 4-Cl—Ph | CH₃ | Y2 |
| 142 | 2-OCH3—Ph | 4-F—Ph | CH₃ | Y2 |
| 143 | 2-OCH3—Ph | 4-CN—Ph | CH₃ | Y2 |
| 144 | 2-OCH3—Ph | 4-OCH3—Ph | CH₃ | Y2 |
| 145 | 2-Cl—Ph | 4-Cl—Ph | CH₃ | Y2 |
| 146 | 2-Cl—Ph | 4-F—Ph | CH₃ | Y2 |
| 147 | 2-Cl—Ph | 4-CN—Ph | CH₃ | Y2 |
| 148 | 2-Cl—Ph | 4-OCH3—Ph | CH₃ | Y2 |
| 149 | 2-OCH3—Ph | 4-Cl—Ph | CH₃ | Y3 |
| 150 | 2-OCH3—Ph | 4-F—Ph | CH₃ | Y3 |
| 151 | 2-OCH3—Ph | 4-CN—Ph | CH₃ | Y3 |
| 152 | 2-OCH3—Ph | 4-OCH3—Ph | CH₃ | Y3 |
| 153 | 2-Cl—Ph | 4-Cl—Ph | CH₃ | Y3 |
| 154 | 2-Cl—Ph | 4-F—Ph | CH₃ | Y3 |
| 155 | 2-Cl—Ph | 4-CN—Ph | CH₃ | Y3 |
| 156 | 2-Cl—Ph | 4-OCH3—Ph | CH₃ | Y3 |
| 157 | 2-OCH3—Ph | 4-Cl—Ph | CH₃ | Y14 |
| 158 | 2-OCH3—Ph | 4-F—Ph | CH₃ | Y14 |
| 159 | 2-OCH3—Ph | 4-CN—Ph | CH₃ | Y14 |
| 160 | 2-OCH3—Ph | 4-OCH3—Ph | CH₃ | Y14 |
| 161 | 2-Cl—Ph | 4-Cl—Ph | CH₃ | Y14 |
| 162 | 2-Cl—Ph | 4-F—Ph | CH₃ | Y14 |
| 163 | 2-Cl—Ph | 4-CN—Ph | CH₃ | Y14 |
| 164 | 2-Cl—Ph | 4-OCH3—Ph | CH₃ | Y14 |
| 165 | 2-OCH3—Ph | 4-Cl—Ph | CH₃ | Y4 |
| 166 | 2-OCH3—Ph | 4-F—Ph | CH₃ | Y4 |
| 167 | 2-OCH3—Ph | 4-CN—Ph | CH₃ | Y4 |
| 168 | 2-OCH3—Ph | 4-OCH3—Ph | CH₃ | Y4 |
| 169 | 2-Cl—Ph | 4-Cl—Ph | CH₃ | Y4 |
| 170 | 2-Cl—Ph | 4-F—Ph | CH₃ | Y4 |
| 171 | 2-Cl—Ph | 4-CN—Ph | CH₃ | Y4 |
| 172 | 2-Cl—Ph | 4-OCH3—Ph | CH₃ | Y4 |
| 173 | 2-OCH3—Ph | 4-Cl—Ph | CH₃ | Y15 |
| 174 | 2-OCH3—Ph | 4-F—Ph | CH₃ | Y15 |
| 175 | 2-OCH3—Ph | 4-CN—Ph | CH₃ | Y15 |
| 176 | 2-OCH3—Ph | 4-OCH3—Ph | CH₃ | Y15 |
| 177 | 2-Cl—Ph | 4-Cl—Ph | CH₃ | Y15 |
| 178 | 2-Cl—Ph | 4-F—Ph | CH₃ | Y15 |
| 179 | 2-Cl—Ph | 4-CN—Ph | CH₃ | Y15 |
| 180 | 2-Cl—Ph | 4-OCH3—Ph | CH₃ | Y15 |
| 181 | 2-OCH3—Ph | 4-Cl—Ph | CH₃ | Y5 |
| 182 | 2-OCH3—Ph | 4-F—Ph | CH₃ | Y5 |
| 183 | 2-OCH3—Ph | 4-CN—Ph | CH₃ | Y5 |
| 184 | 2-OCH3—Ph | 4-OCH3—Ph | CH₃ | Y5 |
| 185 | 2-Cl—Ph | 4-Cl—Ph | CH₃ | Y5 |
| 186 | 2-Cl—Ph | 4-F—Ph | CH₃ | Y5 |
| 187 | 2-Cl—Ph | 4-CN—Ph | CH₃ | Y5 |
| 188 | 2-Cl—Ph | 4-OCH3—Ph | CH₃ | Y5 |
| 189 | 2-OCH3—Ph | 4-Cl—Ph | CH₃ | Y13 |
| 190 | 2-OCH3—Ph | 4-F—Ph | CH₃ | Y13 |
| 191 | 2-OCH3—Ph | 4-CN—Ph | CH₃ | Y13 |
| 192 | 2-OCH3—Ph | 4-OCH3—Ph | CH₃ | Y13 |
| 193 | 2-Cl—Ph | 4-Cl—Ph | CH₃ | Y13 |
| 194 | 2-Cl—Ph | 4-F—Ph | CH₃ | Y13 |
| 195 | 2-Cl—Ph | 4-CN—Ph | CH₃ | Y13 |
| 196 | 2-Cl—Ph | 4-OCH3—Ph | CH₃ | Y13 |
| 197 | 2-OCH3—Ph | 4-Cl—Ph | CH₃ | Y6 |
| 198 | 2-OCH3—Ph | 4-F—Ph | CH₃ | Y6 |
| 199 | 2-OCH3—Ph | 4-CN—Ph | CH₃ | Y6 |
| 200 | 2-OCH3—Ph | 4-OCH3—Ph | CH₃ | Y6 |
| 201 | 2-Cl—Ph | 4-Cl—Ph | CH₃ | Y6 |
| 202 | 2-Cl—Ph | 4-F—Ph | CH₃ | Y6 |
| 203 | 2-Cl—Ph | 4-CN—Ph | CH₃ | Y6 |
| 204 | 2-Cl—Ph | 4-OCH3—Ph | CH₃ | Y6 |
| 205 | 2-OCH3—Ph | 4-Cl—Ph | CH₃ | Y16 |
| 206 | 2-OCH3—Ph | 4-F—Ph | CH₃ | Y16 |
| 207 | 2-OCH3—Ph | 4-CN—Ph | CH₃ | Y16 |
| 208 | 2-OCH3—Ph | 4-OCH3—Ph | CH₃ | Y16 |
| 209 | 2-Cl—Ph | 4-Cl—Ph | CH₃ | Y16 |
| 210 | 2-Cl—Ph | 4-F—Ph | CH₃ | Y16 |
| 211 | 2-Cl—Ph | 4-CN—Ph | CH₃ | Y16 |
| 212 | 2-Cl—Ph | 4-OCH3—Ph | CH₃ | Y16 |
| 213 | 2-OCH3—Ph | 4-Cl—Ph | CH₃ | Y7 |
| 214 | 2-OCH3—Ph | 4-F—Ph | CH₃ | Y7 |
| 215 | 2-OCH3—Ph | 4-CN—Ph | CH₃ | Y7 |
| 216 | 2-OCH3—Ph | 4-OCH3—Ph | CH₃ | Y7 |
| 217 | 2-Cl—Ph | 4-Cl—Ph | CH₃ | Y7 |
| 218 | 2-Cl—Ph | 4-F—Ph | CH₃ | Y7 |
| 219 | 2-Cl—Ph | 4-CN—Ph | CH₃ | Y7 |
| 220 | 2-Cl—Ph | 4-OCH3—Ph | CH₃ | Y7 |
| 221 | 2-OCH3—Ph | 4-Cl—Ph | CH₃ | Y17 |
| 222 | 2-OCH3—Ph | 4-F—Ph | CH₃ | Y17 |
| 223 | 2-OCH3—Ph | 4-CN—Ph | CH₃ | Y17 |
| 224 | 2-OCH3—Ph | 4-OCH3—Ph | CH₃ | Y17 |
| 225 | 2-Cl—Ph | 4-Cl—Ph | CH₃ | Y17 |
| 226 | 2-Cl—Ph | 4-F—Ph | CH₃ | Y17 |
| 227 | 2-Cl—Ph | 4-CN—Ph | CH₃ | Y17 |
| 228 | 2-Cl—Ph | 4-OCH3—Ph | CH₃ | Y17 |
| 229 | 2-OCH3—Ph | 4-Cl—Ph | CH₃ | Y11 |
| 230 | 2-OCH3—Ph | 4-F—Ph | CH₃ | Y11 |
| 231 | 2-OCH3—Ph | 4-CN—Ph | CH₃ | Y11 |
| 232 | 2-OCH3—Ph | 4-OCH3—Ph | CH₃ | Y11 |
| 233 | 2-Cl—Ph | 4-Cl—Ph | CH₃ | Y11 |
| 234 | 2-Cl—Ph | 4-F—Ph | CH₃ | Y11 |
| 235 | 2-Cl—Ph | 4-CN—Ph | CH₃ | Y11 |
| 236 | 2-Cl—Ph | 4-OCH3—Ph | CH₃ | Y11 |
| 237 | 2-OCH3—Ph | 4-Cl—Ph | CH₃ | Y12 |
| 238 | 2-OCH3—Ph | 4-F—Ph | CH₃ | Y12 |
| 239 | 2-OCH3—Ph | 4-CN—Ph | CH₃ | Y12 |
| 240 | 2-OCH3—Ph | 4-OCH3—Ph | CH₃ | Y12 |
| 241 | 2-Cl—Ph | 4-Cl—Ph | CH₃ | Y12 |
| 242 | 2-Cl—Ph | 4-F—Ph | CH₃ | Y12 |
| 243 | 2-Cl—Ph | 4-CN—Ph | CH₃ | Y12 |
| 244 | 2-Cl—Ph | 4-OCH3—Ph | CH₃ | Y12 |
| 245 | 2-OCH3—Ph | 4-Cl—Ph | OCH₃ | Y1 |
| 246 | 2-OCH3—Ph | 4-F—Ph | OCH₃ | Y1 |
| 247 | 2-OCH3—Ph | 4-CN—Ph | OCH₃ | Y1 |
| 248 | 2-OCH3—Ph | 4-OCH3—Ph | OCH₃ | Y1 |
| 249 | 2-Cl—Ph | 4-Cl—Ph | OCH₃ | Y1 |
| 250 | 2-Cl—Ph | 4-F—Ph | OCH₃ | Y1 |
| 251 | 2-Cl—Ph | 4-CN—Ph | OCH₃ | Y1 |
| 252 | 2-Cl—Ph | 4-OCH3—Ph | OCH₃ | Y1 |
| 253 | 2-OCH3—Ph | 4-Cl—Ph | OCH₃ | Y2 |
| 254 | 2-OCH3—Ph | 4-F—Ph | OCH₃ | Y2 |
| 255 | 2-OCH3—Ph | 4-CN—Ph | OCH₃ | Y2 |
| 266 | 2-OCH3—Ph | 4-OCH3—Ph | OCH₃ | Y2 |
| 267 | 2-Cl—Ph | 4-Cl—Ph | OCH₃ | Y2 |
| 268 | 2-Cl—Ph | 4-F—Ph | OCH₃ | Y2 |
| 269 | 2-Cl—Ph | 4-CN—Ph | OCH₃ | Y2 |
| 270 | 2-Cl—Ph | 4-OCH3—Ph | OCH₃ | Y2 |
| 271 | 2-OCH3—Ph | 4-Cl—Ph | OCH₃ | Y3 |
| 272 | 2-OCH3—Ph | 4-F—Ph | OCH₃ | Y3 |
| 273 | 2-OCH3—Ph | 4-CN—Ph | OCH₃ | Y3 |
| 274 | 2-OCH3—Ph | 4-OCH3—Ph | OCH₃ | Y3 |
| 275 | 2-Cl—Ph | 4-Cl—Ph | OCH₃ | Y3 |
| 276 | 2-Cl—Ph | 4-F—Ph | OCH₃ | Y3 |
| 277 | 2-Cl—Ph | 4-CN—Ph | OCH₃ | Y3 |
| 278 | 2-Cl—Ph | 4-OCH3—Ph | OCH₃ | Y3 |
| 279 | 2-OCH3—Ph | 4-Cl—Ph | OCH₃ | Y14 |
| 280 | 2-OCH3—Ph | 4-F—Ph | OCH₃ | Y14 |
| 281 | 2-OCH3—Ph | 4-CN—Ph | OCH₃ | Y14 |
| 282 | 2-OCH3—Ph | 4-OCH3—Ph | OCH₃ | Y14 |
| 283 | 2-Cl—Ph | 4-Cl—Ph | OCH₃ | Y14 |
| 284 | 2-Cl—Ph | 4-F—Ph | OCH₃ | Y14 |
| 285 | 2-Cl—Ph | 4-CN—Ph | OCH₃ | Y14 |
| 286 | 2-Cl—Ph | 4-OCH3—Ph | OCH₃ | Y14 |
| 287 | 2-OCH3—Ph | 4-Cl—Ph | OCH₃ | Y4 |
| 288 | 2-OCH3—Ph | 4-F—Ph | OCH₃ | Y4 |
| 289 | 2-OCH3—Ph | 4-CN—Ph | OCH₃ | Y4 |
| 290 | 2-OCH3—Ph | 4-OCH3—Ph | OCH₃ | Y4 |
| 291 | 2-Cl—Ph | 4-Cl—Ph | OCH₃ | Y4 |
| 292 | 2-Cl—Ph | 4-F—Ph | OCH₃ | Y4 |
| 293 | 2-Cl—Ph | 4-CN—Ph | OCH₃ | Y4 |
| 294 | 2-Cl—Ph | 4-OCH3—Ph | OCH₃ | Y4 |
| 295 | 2-OCH3—Ph | 4-Cl—Ph | OCH₃ | Y15 |
| 296 | 2-OCH3—Ph | 4-F—Ph | OCH₃ | Y15 |
| 297 | 2-OCH3—Ph | 4-CN—Ph | OCH₃ | Y15 |
| 298 | 2-OCH3—Ph | 4-OCH3—Ph | OCH₃ | Y15 |
| 299 | 2-Cl—Ph | 4-Cl—Ph | OCH₃ | Y15 |
| 300 | 2-Cl—Ph | 4-F—Ph | OCH₃ | Y15 |
| 301 | 2-Cl—Ph | 4-CN—Ph | OCH₃ | Y15 |
| 302 | 2-Cl—Ph | 4-OCH3—Ph | OCH₃ | Y15 |
| 303 | 2-OCH3—Ph | 4-Cl—Ph | OCH₃ | Y5 |
| 304 | 2-OCH3—Ph | 4-F—Ph | OCH₃ | Y5 |
| 305 | 2-OCH3—Ph | 4-CN—Ph | OCH₃ | Y5 |
| 306 | 2-OCH3—Ph | 4-OCH3—Ph | OCH₃ | Y5 |
| 307 | 2-Cl—Ph | 4-Cl—Ph | OCH₃ | Y5 |
| 308 | 2-Cl—Ph | 4-F—Ph | OCH₃ | Y5 |
| 309 | 2-Cl—Ph | 4-CN—Ph | OCH₃ | Y5 |
| 310 | 2-Cl—Ph | 4-OCH3—Ph | OCH₃ | Y5 |
| 311 | 2-OCH3—Ph | 4-Cl—Ph | OCH₃ | Y13 |
| 312 | 2-OCH3—Ph | 4-F—Ph | OCH₃ | Y13 |
| 313 | 2-OCH3—Ph | 4-CN—Ph | OCH₃ | Y13 |
| 314 | 2-OCH3—Ph | 4-OCH3—Ph | OCH₃ | Y13 |
| 315 | 2-Cl—Ph | 4-Cl—Ph | OCH₃ | Y13 |
| 316 | 2-Cl—Ph | 4-F—Ph | OCH₃ | Y13 |
| 317 | 2-Cl—Ph | 4-CN—Ph | OCH₃ | Y13 |
| 318 | 2-Cl—Ph | 4-OCH3—Ph | OCH₃ | Y13 |
| 319 | 2-OCH3—Ph | 4-Cl—Ph | OCH₃ | Y6 |
| 320 | 2-OCH3—Ph | 4-F—Ph | OCH₃ | Y6 |
| 321 | 2-OCH3—Ph | 4-CN—Ph | OCH₃ | Y6 |
| 322 | 2-OCH3—Ph | 4-OCH3—Ph | OCH₃ | Y6 |
| 323 | 2-Cl—Ph | 4-Cl—Ph | OCH₃ | Y6 |
| 324 | 2-Cl—Ph | 4-F—Ph | OCH₃ | Y6 |
| 325 | 2-Cl—Ph | 4-CN—Ph | OCH₃ | Y6 |
| 326 | 2-Cl—Ph | 4-OCH3—Ph | OCH₃ | Y6 |
| 327 | 2-OCH3—Ph | 4-Cl—Ph | OCH₃ | Y16 |
| 328 | 2-OCH3—Ph | 4-F—Ph | OCH₃ | Y16 |
| 329 | 2-OCH3—Ph | 4-CN—Ph | OCH₃ | Y16 |
| 330 | 2-OCH3—Ph | 4-OCH3—Ph | OCH₃ | Y16 |
| 331 | 2-Cl—Ph | 4-Cl—Ph | OCH₃ | Y16 |
| 332 | 2-Cl—Ph | 4-F—Ph | OCH₃ | Y16 |
| 333 | 2-Cl—Ph | 4-CN—Ph | OCH₃ | Y16 |
| 334 | 2-Cl—Ph | 4-OCH3—Ph | OCH₃ | Y16 |
| 335 | 2-OCH3—Ph | 4-Cl—Ph | OCH₃ | Y7 |
| 336 | 2-OCH3—Ph | 4-F—Ph | OCH₃ | Y7 |
| 337 | 2-OCH3—Ph | 4-CN—Ph | OCH₃ | Y7 |
| 338 | 2-OCH3—Ph | 4-OCH3—Ph | OCH₃ | Y7 |
| 339 | 2-Cl—Ph | 4-Cl—Ph | OCH₃ | Y7 |
| 340 | 2-Cl—Ph | 4-F—Ph | OCH₃ | Y7 |
| 341 | 2-Cl—Ph | 4-CN—Ph | OCH₃ | Y7 |
| 342 | 2-Cl—Ph | 4-OCH3—Ph | OCH₃ | Y7 |
| 343 | 2-OCH3—Ph | 4-Cl—Ph | OCH₃ | Y17 |
| 344 | 2-OCH3—Ph | 4-F—Ph | OCH₃ | Y17 |
| 345 | 2-OCH3—Ph | 4-CN—Ph | OCH₃ | Y17 |
| 346 | 2-OCH3—Ph | 4-OCH3—Ph | OCH₃ | Y17 |
| 347 | 2-Cl—Ph | 4-Cl—Ph | OCH₃ | Y17 |
| 348 | 2-Cl—Ph | 4-F—Ph | OCH₃ | Y17 |
| 349 | 2-Cl—Ph | 4-CN—Ph | OCH₃ | Y17 |
| 350 | 2-Cl—Ph | 4-OCH3—Ph | OCH₃ | Y17 |
| 351 | 2-OCH3—Ph | 4-Cl—Ph | OCH₃ | Y11 |
| 352 | 2-OCH3—Ph | 4-F—Ph | OCH₃ | Y11 |
| 353 | 2-OCH3—Ph | 4-CN—Ph | OCH₃ | Y11 |
| 354 | 2-OCH3—Ph | 4-OCH3—Ph | OCH₃ | Y11 |
| 355 | 2-Cl—Ph | 4-Cl—Ph | OCH₃ | Y11 |
| 356 | 2-Cl—Ph | 4-F—Ph | OCH₃ | Y11 |
| 357 | 2-Cl—Ph | 4-CN—Ph | OCH₃ | Y11 |
| 358 | 2-Cl—Ph | 4-OCH3—Ph | OCH₃ | Y11 |
| 359 | 2-OCH3—Ph | 4-Cl—Ph | OCH₃ | Y12 |
| 360 | 2-OCH3—Ph | 4-F—Ph | OCH₃ | Y12 |
| 361 | 2-OCH3—Ph | 4-CN—Ph | OCH₃ | Y12 |
| 362 | 2-OCH3—Ph | 4-OCH3—Ph | OCH₃ | Y12 |
| 363 | 2-Cl—Ph | 4-Cl—Ph | OCH₃ | Y12 |
| 364 | 2-Cl—Ph | 4-F—Ph | OCH₃ | Y12 |
| 365 | 2-Cl—Ph | 4-CN—Ph | OCH₃ | Y12 |
| 366 | 2-Cl—Ph | 4-OCH3—Ph | OCH₃ | Y12 |
| 367 | 2-OCH3—Ph | 4-Cl—Ph | Cl | Y1 |
| 368 | 2-OCH3—Ph | 4-F—Ph | Cl | Y1 |
| 369 | 2-OCH3—Ph | 4-CN—Ph | Cl | Y1 |
| 370 | 2-OCH3—Ph | 4-OCH3—Ph | Cl | Y1 |
| 371 | 2-Cl—Ph | 4-Cl—Ph | Cl | Y1 |
| 372 | 2-Cl—Ph | 4-F—Ph | Cl | Y1 |
| 373 | 2-Cl—Ph | 4-CN—Ph | Cl | Y1 |
| 374 | 2-Cl—Ph | 4-OCH3—Ph | Cl | Y1 |
| 375 | 2-OCH3—Ph | 4-Cl—Ph | Cl | Y2 |
| 376 | 2-OCH3—Ph | 4-F—Ph | Cl | Y2 |
| 377 | 2-OCH3—Ph | 4-CN—Ph | Cl | Y2 |
| 378 | 2-OCH3—Ph | 4-OCH3—Ph | Cl | Y2 |
| 379 | 2-Cl—Ph | 4-Cl—Ph | Cl | Y2 |
| 380 | 2-Cl—Ph | 4-F—Ph | Cl | Y2 |
| 381 | 2-Cl—Ph | 4-CN—Ph | Cl | Y2 |
| 382 | 2-Cl—Ph | 4-OCH3—Ph | Cl | Y2 |
| 383 | 2-OCH3—Ph | 4-Cl—Ph | Cl | Y3 |
| 384 | 2-OCH3—Ph | 4-F—Ph | Cl | Y3 |
| 385 | 2-OCH3—Ph | 4-CN—Ph | Cl | Y3 |
| 386 | 2-OCH3—Ph | 4-OCH3—Ph | Cl | Y3 |
| 387 | 2-Cl—Ph | 4-Cl—Ph | Cl | Y3 |
| 388 | 2-Cl—Ph | 4-F—Ph | Cl | Y3 |
| 389 | 2-Cl—Ph | 4-CN—Ph | Cl | Y3 |
| 390 | 2-Cl—Ph | 4-OCH3—Ph | Cl | Y3 |
| 391 | 2-OCH3—Ph | 4-Cl—Ph | Cl | Y14 |
| 392 | 2-OCH3—Ph | 4-F—Ph | Cl | Y14 |
| 393 | 2-OCH3—Ph | 4-CN—Ph | Cl | Y14 |
| 394 | 2-OCH3—Ph | 4-OCH3—Ph | Cl | Y14 |
| 395 | 2-Cl—Ph | 4-Cl—Ph | Cl | Y14 |
| 396 | 2-Cl—Ph | 4-F—Ph | Cl | Y14 |
| 397 | 2-Cl—Ph | 4-CN—Ph | Cl | Y14 |
| 398 | 2-Cl—Ph | 4-OCH3—Ph | Cl | Y14 |
| 399 | 2-OCH3—Ph | 4-Cl—Ph | Cl | Y4 |
| 400 | 2-OCH3—Ph | 4-F—Ph | Cl | Y4 |
| 401 | 2-OCH3—Ph | 4-CN—Ph | Cl | Y4 |
| 402 | 2-OCH3—Ph | 4-OCH3—Ph | Cl | Y4 |
| 403 | 2-Cl—Ph | 4-Cl—Ph | Cl | Y4 |
| 404 | 2-Cl—Ph | 4-F—Ph | Cl | Y4 |
| 405 | 2-Cl—Ph | 4-CN—Ph | Cl | Y4 |
| 406 | 2-Cl—Ph | 4-OCH3—Ph | Cl | Y4 |
| 407 | 2-OCH3—Ph | 4-Cl—Ph | Cl | Y15 |
| 408 | 2-OCH3—Ph | 4-F—Ph | Cl | Y15 |
| 409 | 2-OCH3—Ph | 4-CN—Ph | Cl | Y15 |
| 410 | 2-OCH3—Ph | 4-OCH3—Ph | Cl | Y15 |
| 411 | 2-Cl—Ph | 4-Cl—Ph | Cl | Y15 |
| 412 | 2-Cl—Ph | 4-F—Ph | Cl | Y15 |
| 413 | 2-Cl—Ph | 4-CN—Ph | Cl | Y15 |
| 414 | 2-Cl—Ph | 4-OCH3—Ph | Cl | Y15 |
| 415 | 2-OCH3—Ph | 4-Cl—Ph | Cl | Y5 |
| 416 | 2-OCH3—Ph | 4-F—Ph | Cl | Y5 |
| 417 | 2-OCH3—Ph | 4-CN—Ph | Cl | Y5 |
| 418 | 2-OCH3—Ph | 4-OCH3—Ph | Cl | Y5 |
| 419 | 2-Cl—Ph | 4-Cl—Ph | Cl | Y5 |
| 420 | 2-Cl—Ph | 4-F—Ph | Cl | Y5 |
| 421 | 2-Cl—Ph | 4-CN—Ph | Cl | Y5 |
| 422 | 2-Cl—Ph | 4-OCH3—Ph | Cl | Y5 |
| 423 | 2-OCH3—Ph | 4-Cl—Ph | Cl | Y13 |
| 424 | 2-OCH3—Ph | 4-F—Ph | Cl | Y13 |
| 425 | 2-OCH3—Ph | 4-CN—Ph | Cl | Y13 |
| 426 | 2-OCH3—Ph | 4-OCH3—Ph | Cl | Y13 |
| 427 | 2-Cl—Ph | 4-Cl—Ph | Cl | Y13 |
| 428 | 2-Cl—Ph | 4-F—Ph | Cl | Y13 |
| 429 | 2-Cl—Ph | 4-CN—Ph | Cl | Y13 |
| 430 | 2-Cl—Ph | 4-OCH3—Ph | Cl | Y13 |
| 431 | 2-OCH3—Ph | 4-Cl—Ph | Cl | Y6 |
| 432 | 2-OCH3—Ph | 4-F—Ph | Cl | Y6 |
| 433 | 2-OCH3—Ph | 4-CN—Ph | Cl | Y6 |
| 434 | 2-OCH3—Ph | 4-OCH3—Ph | Cl | Y6 |
| 435 | 2-Cl—Ph | 4-Cl—Ph | Cl | Y6 |
| 436 | 2-Cl—Ph | 4-F—Ph | Cl | Y6 |
| 437 | 2-Cl—Ph | 4-CN—Ph | Cl | Y6 |
| 438 | 2-Cl—Ph | 4-OCH3—Ph | Cl | Y6 |
| 439 | 2-OCH3—Ph | 4-Cl—Ph | Cl | Y16 |
| 440 | 2-OCH3—Ph | 4-F—Ph | Cl | Y16 |
| 441 | 2-OCH3—Ph | 4-CN—Ph | Cl | Y16 |
| 442 | 2-OCH3—Ph | 4-OCH3—Ph | Cl | Y16 |
| 443 | 2-Cl—Ph | 4-Cl—Ph | Cl | Y16 |
| 444 | 2-Cl—Ph | 4-F—Ph | Cl | Y16 |
| 445 | 2-Cl—Ph | 4-CN—Ph | Cl | Y16 |
| 446 | 2-Cl—Ph | 4-OCH3—Ph | Cl | Y16 |
| 447 | 2-OCH3—Ph | 4-Cl—Ph | Cl | Y7 |
| 448 | 2-OCH3—Ph | 4-F—Ph | Cl | Y7 |
| 449 | 2-OCH3—Ph | 4-CN—Ph | Cl | Y7 |
| 450 | 2-OCH3—Ph | 4-OCH3—Ph | Cl | Y7 |
| 451 | 2-Cl—Ph | 4-Cl—Ph | Cl | Y7 |
| 452 | 2-Cl—Ph | 4-F—Ph | Cl | Y7 |
| 453 | 2-Cl—Ph | 4-CN—Ph | Cl | Y7 |
| 454 | 2-Cl—Ph | 4-OCH3—Ph | Cl | Y7 |
| 455 | 2-OCH3—Ph | 4-Cl—Ph | Cl | Y17 |
| 456 | 2-OCH3—Ph | 4-F—Ph | Cl | Y17 |
| 457 | 2-OCH3—Ph | 4-CN—Ph | Cl | Y17 |
| 458 | 2-OCH3—Ph | 4-OCH3—Ph | Cl | Y17 |
| 459 | 2-Cl—Ph | 4-Cl—Ph | Cl | Y17 |
| 460 | 2-Cl—Ph | 4-F—Ph | Cl | Y17 |
| 461 | 2-Cl—Ph | 4-CN—Ph | Cl | Y17 |
| 462 | 2-Cl—Ph | 4-OCH3—Ph | Cl | Y17 |
| 463 | 2-OCH3—Ph | 4-Cl—Ph | Cl | Y11 |
| 464 | 2-OCH3—Ph | 4-F—Ph | Cl | Y11 |
| 465 | 2-OCH3—Ph | 4-CN—Ph | Cl | Y11 |
| 466 | 2-OCH3—Ph | 4-OCH3—Ph | Cl | Y11 |
| 467 | 2-Cl—Ph | 4-Cl—Ph | Cl | Y11 |
| 468 | 2-Cl—Ph | 4-F—Ph | Cl | Y11 |
| 469 | 2-Cl—Ph | 4-CN—Ph | Cl | Y11 |
| 470 | 2-Cl—Ph | 4-OCH3—Ph | Cl | Y11 |
| 471 | 2-OCH3—Ph | 4-Cl—Ph | Cl | Y12 |
| 472 | 2-OCH3—Ph | 4-F—Ph | Cl | Y12 |
| 473 | 2-OCH3—Ph | 4-CN—Ph | Cl | Y12 |
| 474 | 2-OCH3—Ph | 4-OCH3—Ph | Cl | Y12 |
| 475 | 2-Cl—Ph | 4-Cl—Ph | Cl | Y12 |
| 476 | 2-Cl—Ph | 4-F—Ph | Cl | Y12 |
| 477 | 2-Cl—Ph | 4-CN—Ph | Cl | Y12 |
| 478 | 2-Cl—Ph | 4-OCH3—Ph | Cl | Y12 |

### Methods for Determining the Biological Activity

### Vasopressin V1b Receptor Binding Assay:

### Substances:

The test substances were dissolved in a concentration of 10⁻² M in DMSO and further diluted to 5×10⁻⁴ M to 5×10⁻⁹ M in DMSO. These DMSO solutions were diluted 1:10 with assay buffer. The substance concentration was again diluted 1:5 in the assay mixture.

### Membrane Preparation:

CHO-K1 cells with stably expressed human vasopressin V1b receptor (clone 3H2) were harvested and homogenized in 50 mM Tris-HCl and in the presence of protease inhibitors (Roche complete Mini #1836170) with a Polytron homogenizer at a medium setting for 2×10 seconds and subsequently centrifuged at 40 000×g for 1 h. The membrane pellet was again homogenized and centrifuged as described and then taken up in 50 mM Tris-HCl, pH 7.4, homogenized and stored in aliquots frozen in liquid nitrogen at −190° C.

### Binding Assay:

The binding assay was carried out by a method based on that of Tahara et al. (Tahara A et al., Brit. J. Pharmacol. 125, 1463-1470 (1998)). The incubation buffer was: 50 mM Tris, 10 mM MgCl₂, 0.1% BSA, pH 7.4.

In the assay mixture (250 μl), membranes (50 μg/ml protein in incubation buffer) from CHO-K₁ cells with stably expressed human V1b receptors (cell line hV1b_{—}3H2_CHO) were incubated with 1.5 nM ³H-AVP (8-Arg-vasopressin, PerkinElmer #18479) in incubation buffer (50 mM Tris, 10 mM MgCl₂, 0.1% BSA, pH 7.4) (total binding) or additionally with increasing concentrations of test substance (displacement experiment). The nonspecific binding was determined with 1 uM AVP (Bachem # H1780). All determinations were carried out as triplicate determinations. After incubation (60 minutes at room temperature), the free radioligand was removed by vacuum filtration (Skatron cell harvester 7000) through Wathman G F/B glass fiber filter mats, and the filters were transferred into scintillation vials. The liquid scintillation measurement took place in a Tricarb model 2000 or 2200CA instrument (Packard). Conversion of the measured cpm into dpm was carried out with the aid of a standard quench series.

### Evaluation:

The binding parameters were calculated by nonlinear regression in SAS. The algorithms of the program operate in analogy to the LIGAND analysis program (Munson P J and Rodbard D, Analytical Biochem. 107, 220-239 (1980)). The Kd of ³H-AVP for the recombinant hV2 receptors is 0.4 nM and was used to determine the Ki value.

### Vasopressin V1a Receptor Binding Assay:

### Substances:

The test substances were dissolved in a concentration of 10⁻² M in DMSO. These DMSO solutions were further diluted in incubation buffer (50 mM Tris, 10 mM MgCl₂, 0.1% BSA, pH 7.4).

### Membrane Preparation:

CHO-K1 cells with stably expressed human vasopressin V1a receptor (clone 5) were harvested and homogenized in 50 mM Tris-HCl and in the presence of protease inhibitors (Roche complete Mini #1836170) with a Polytron homogenizer at a medium setting for 2×10 seconds and subsequently centrifuged at 40 000×g for 1 h. The membrane pellet was again homogenized and centrifuged as described and then taken up in 50 mM Tris-HCl, pH 7.4, homogenized and stored in aliquots frozen in liquid nitrogen at −190° C.

Binding Assay:

The binding assay was carried out by a method based on that of Tahara et al. (Tahara A et al., Brit. J. Pharmacol. 125, 1463-1470 (1998)).

The incubation buffer was: 50 mM Tris, 10 mM MgCl₂, 0.1% BSA, pH 7.4.

In the assay mixture (250 μl), membranes (20 μg/ml protein in incubation buffer) from CHO-K1 cells with stably expressed human V1a receptors (cell line hV1a_{—}5_CHO) were incubated with 0.04 nM ¹²⁵I-AVP (8-Arg-vasopressin, NEX 128) in incubation buffer (50 mM Tris, 10 mM MgCl₂, 0.1% BSA, pH 7.4) (total binding) or additionally with increasing concentrations of test substance (displacement experiment). The nonspecific binding was determined with 1 μM AVP (Bachem #H1780). Triplicate determinations were carried out.

After incubation (60 minutes at room temperature), the free radioligand was removed by vacuum filtration (Skatron cell harvester 7000) through Wathman GF/B glass fiber filter mats, and the filters were transferred into scintillation vials. The liquid scintillation measurement took place in a Tricarb model 2000 or 2200CA instrument (Packard). Conversion of the measured cpm into dpm was carried out with the aid of a standard quench series.

### Evaluation:

The binding parameters were calculated by nonlinear regression in SAS. The algorithms of the program operate in analogy to the LIGAND analysis program (Munson P J and Rodbard D, Analytical Biochem. 107, 220-239 (1980)). The Kd of ¹²⁵I-AVP for the recombinant hV1a receptors was determined in saturation experiments. A Kd of 1.33 nM was used to determine the Ki value.

### Vasopressin V2 Receptor Binding Assay:

### Substances:

The test substances were dissolved in a concentration of 10⁻² M in DMSO and further diluted to 10⁻³ M to 5×10⁻⁹ M in DMSO. These DMSO solutions were further diluted in incubation buffer (50 mM Tris, 10 mM MgCl₂, 0.1% BSA, pH 7.4).

### Membrane Preparation:

CHO-K1 cells with stably expressed human vasopressin V2 receptor (clone 23) were harvested and homogenized in 50 mM Tris-HCl and in the presence of protease inhibitors (Roche complete Mini #1836170) with a Polytron homogenizer at a medium setting for 2×10 seconds and subsequently centrifuged at 40 000×g for 1 h. The membrane pellet was again homogenized and centrifuged as described and then taken up in 50 mM Tris-HCl, pH 7.4, homogenized and stored in aliquots frozen in liquid nitrogen at −190° C.

Binding Assay:

The binding assay was carried out by a method based on that of Tahara et al. (Tahara A et al., Brit. J. Pharmacol. 125, 1463-1470 (1998)).

The incubation buffer was: 50 mM Tris, 10 mM MgCl₂, 0.1% BSA, pH 7.4.

In the assay mixture (250 μl), membranes (50 μg/ml protein in incubation buffer) from CHO-K1 cells with stably expressed human V2 receptors (cell line hV2_{—}23_CHO) were incubated with 1-2 nM ³H-AVP (8-Arg-vasopressin, PerkinElmer #18479) in incubation buffer (50 mM Tris, 10 mM MgCl₂, 0.1% BSA, pH 7.4) (total binding) or additionally with increasing concentrations of test substance (displacement experiment). The nonspecific binding was determined with 1 μM AVP (Bachem #H1780). Triplicate determinations were carried out.

After incubation (60 minutes at room temperature), the free radioligand was removed by vacuum filtration (Skatron cell harvester 7000) through Wathman GF/B glass fiber filter mats, and the filters were transferred into scintillation vials. The liquid scintillation measurement took place in a Tricarb model 2000 or 2200CA instrument (Packard). Conversion of the measured cpm into dpm was carried out with the aid of a standard quench series.

### Evaluation:

The binding parameters were calculated by nonlinear regression in SAS. The algorithms of the program operate in analogy to the LIGAND analysis program (Munson P J and Rodbard D, Analytical Biochem. 107, 220-239 (1980)). The Kd of ³H-AVP for the recombinant hV1b receptors is 2.4 nM and was used to determine the Ki value.

### Oxytocin Receptor Binding Assay

### Substances:

The substances were dissolved in a concentration of 10⁻² M or 10⁻³ M in DMSO and diluted with incubation buffer (50 mM Tris, 10 mM MgCl₂, 0.1% BSA, pH 7.4).

### Cell Preparation:

Confluent HEK-293 cells with transiently expressing recombinant human oxytocin receptors were centrifuged at 750×g and at room temperature for 5 minutes. The residue was taken up in ice-cold lysis buffer (50 mM Tris-HCl, 10% glycerol, pH7.4 and Roche Complete Protease Inhibitor) and subjected to an osmotic shock at 4° C. for 20 minutes. The lysed cells were then centrifuged at 750×g and at 4° C. for 20 minutes, the residue was taken up in incubation buffer, and aliquots of 10⁷ cells/ml were prepared. The aliquots were frozen at −80° C. until used.

### Binding Assay:

On the day of the experiment, the cells were thawed, diluted with incubation buffer and homogenized using a Multipette Combitip (Eppendorf, Hamburg). The reaction mixture of 0.250 ml was composed of 2 to 5×10⁴ recombinant cells, 3-4 nM ³H-oxytocin (PerkinElmer, NET 858) in the presence of test substance (inhibition plot) or only incubation buffer (total binding). The nonspecific binding was determined with 10⁻⁶ M oxytocin (Bachem AG, H2510). Determinations in triplicate were set up. Bound and free radioligand were separated by filtration under vacuum with Whatman GF/B glass fiber filters using a Skatron cell harvester 7000. The bound radioactivity was determined by liquid scintillation measurement in a Tricarb beta counter, model 2000 or 2200CA (Packard).

### Evaluation:

The binding parameters were calculated by nonlinear regression analysis (SAS), in analogy to the LIGAND program of Munson and Rodbard (Analytical Biochem 1980; 107: 220-239). The Kd of ³H-oxytocin for the recombinant hOT receptors is 7.6 nM and was used to determine the Ki value.

### Effect on Vasopressin-Induced Calcium Increase in Cells Having a Cloned Human Vasopressin Receptor

The functional activity of the test substances was investigated on CHO-K1 cells which were stably transfected with the human V1b receptor. 50 000 cells were seeded in each well of a microtiter plate with 96 wells and incubated in culture medium in a saturated water vapor atmosphere with 5% CO₂ at 37° C. overnight. The culture medium consisted of DMEM/Nut Mix F12 with Glutamax I (from Invitrogen), 10% fetal calf serum, 100 units/ml penicillin, 100 μg/ml streptomycin and 800 μg/ml Geneticin. The following day, the cells were washed with culture medium and loaded with a fluorescent dye for calcium in accordance with the manufacturer's statements (Ca⁺⁺-Plus-Assay Kit, Molecular Devices). The cells were loaded in the presence of probenzide (1 vol %). The test substances were diluted with culture medium (final concentration 10⁻¹⁰ to 10⁻⁵M) and incubated with the dye-loaded cells at room temperature for 15 minutes. The Arg-vasopressin (10⁴³M) was added and the maximum fluorescence signal was determined using a FLIPR-96 measuring instrument (Molecular Devices). Concentration-effect plots were constructed using nonlinear regression algorithms (GraphPad Prism 3.0). Kb values were calculated from IC50 values by the method of Cheng and Prusoff (Kb=IC50/1+L/EC50).

The affinities of the compounds of the invention for the human vasopressin V1b receptor were measured in accordance with the above assays, and the affinity constants (Ki) were determined. The Ki values shown therein by examples 1, 15, 16, 17b and 19 were below 100 nM. In addition, the affinities for the vasopressin V1a, V2 receptors and the oxytocin (OT) receptor were determined in accordance with the above assays. It emerged from this that examples 1, 15, 16, 17b and 19 exhibit an improved selectivity vis-6-vis V1b by comparison with V1a, V2 and/or OT (in each case measured as the quotient of the corresponding Ki values, that is “Ki(V1a)/Ki(V1b)”, “Ki(V2)/Ki(v1b)” and/or “Ki(OT)Ki(V1b)”.

1. A compound of the formula (I), 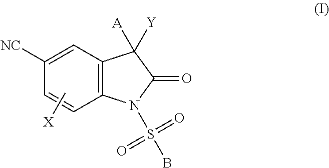
in which
A is C₆-C₁₀-aryl which may be substituted by one, two, three or four radicals selected from the group consisting of R_{A}¹, R_{A}², R_{A}³ and/or R_{A}⁴, in which R_{A}¹, R_{A}², R_{A}³ and R_{A}⁴ are independently of one another and independently of their respective occurrence selected from the group consisting of hydrogen, chlorine, bromine, iodine, fluorine, CN, OR_{A}⁵, COR_{A}⁵, COOR_{A}⁵, SR_{A}⁵, C₃-C₇-cycloalkyl, OCOR_{A}⁵, SO₂NR_{A}⁶R_{A}⁷, CONR_{A}⁶R_{A}⁷, C₀-C₄-alkylene-CN, C₁-C₆-haloalkyl, C₁-C₆-haloalkoxy, NO₂, C₀-C₄-alkylene-OR_{A}⁵, C₁-C₆-alkyl, C₂-C₆-alkenyl, C₂-C₆-alkynyl, C₂-C₆-alkenyloxy, C₂-C₆-alkynyloxy, C₁-C₄-alkylthio, C₀-C₄-alkylene-COR_{A}⁵, SO₂R_{A}⁵,
C₀-C₄-alkylene-COOR_{A}⁵, O—C₁-C₄-alkylene-COOR_{A}⁵, C₀-C₄-alkylene-SR_{A}⁵, C₀-C₄-alkylene-C₃-C₇-cycloalkyl, C₀-C₄-alkylene-OCOR_{A}⁵, C₀-C₄-alkylene-SO₂NR_{A}⁶R_{A}⁷,
C₀-C₄-alkylene-CONR_{A}⁶R_{A}⁷, C₁-C₄-alkylene-OCONR_{A}⁶R_{A}⁷, C₁-C₄-alkylene-SOR_{A}⁵, C₁-C₄-alkylene-SO₂R_{A}⁵, NHCOO—C₀-C₄-alkylene-aryl, NHCOOH, NH₂, NH(C₁-C₄-alkyl), N(C₁-C₄-alkyl)₂,
where two of the radicals R_{A}¹, R_{A}², R_{A}³ and R_{A}⁴ in adjacent position (“ortho”) to one another may also form an optionally substituted, fused saturated, unsaturated and/or aromatic 3- to 10-membered carbocycle or a cyclic acetal —O—CH₂—CH₂—O— or —O—CH₂—O—,
and in which
R_{A}⁵ is independently of its respective occurrence hydrogen, a branched or unbranched radical C₁-C₆-alkyl, or a branched or unbranched, optionally substituted radical C₂-C₆-alkenyl-, C₂-C₆-alkynyl, C₃-C₇-cycloalkyl-, C₁-C₄-alkylene-C₃-C₇-cycloalkyl- or C₁-C₄-alkylene-(C₆-C₁₀)-aryl,
R_{A}⁶ and R_{A}⁷ are independently of one another and independently of their respective occurrence hydrogen, a branched or unbranched, optionally substituted radical C₁-C₆-alkyl-, C₂-C₆-alkenyl-, C₂-C₆-alkynyl, C₁-C₅-alkylene-C₁-C₄-alkoxy-, C₃-C₇-cycloalkyl-, C₁-C₄-alkylene-C₃-C₇-cycloalkyl, C₁-C₄-alkylene-aryl, or a radical —SO₂R_{A}⁵, —CO₂R_{A}⁵, —CO—NR_{A}⁵ R_{A}⁵, or —COR_{A}⁵, and
B is an aromatic or partly aromatic C₆-C₁₀-mono or fused bicycle which may be substituted with a maximum of four radicals selected from the group consisting of R_{B}¹, R_{B}2, R_{B}³, and R_{B}⁴, where R_{B}¹, R_{B}², R_{B}³ and R_{B}⁴ are selected independently of one another and independently of their respective occurrence from the group consisting of hydrogen, chlorine, bromine, iodine, fluorine, CN, OR_{B}⁵, COR_{B}⁵, COOR_{B}⁵, SR_{B}⁵, C₃-C₇-cycloalkyl, OCOR_{A}⁵, SO₂NR_{A}⁶R_{A}⁷, CONR_{A}⁶R_{A}⁷, (C₆-C₁₀)-aryl, (C₃-C₁₀)-hetaryl, NR_{B}⁶R_{B}⁷, C₃-C₇-heterocycloalkyl, C₃-C₇-heterocycloalkenyl, OCOR_{B}⁵, SO₂NR_{B}⁶R_{B}⁷, CONR_{B}⁶R_{B}⁷, C₀-C₄-alkylene-CN, C₁-C₆-haloalkyl, C₁-C₆-haloalkoxy, NO₂, C₀-C₄-alkylene-OR_{B}⁵, O—C₀-C₄-alkylene-(C₆-C₁₀)-aryl, O—C₀-C₄-alkylene-(C₂-C₁₀)-hetaryl, C₀-C₄-alkylene-(C₆-C₁₀)-aryl, C₀-C₄-alkylene-(C₂-C₁₀)— hetaryl, C₁-C₆-alkyl, C₂-C₆-alkenyl, C₂-C₆-alkynyl, C₂-C₆-alkenyloxy, C₂-C₆-alkynyloxy, C₁-C₄-alkylthio, C₀-C₄-alkylene-NR_{B}⁶R_{B}⁷, C₀-C₄-alkylene-COR_{B}⁵, SO₂R_{B}⁵, C₀-C₄-alkylene-COOR_{B}⁵, O—C₁-C₄-alkylene-COOR_{B}⁵, C₀-C₄-alkylene-SR_{B}⁵, C₀-C₄-alkylene-C₃-C₇-cycloalkyl, C₀-C₄-alkylene-C₃-C₇-heterocycloalkyl, C₀-C₄-alkylene-C₃-C₇-heterocycloalkenyl, C₀-C₄-alkylene-OCOR_{B}⁵, C₀-C₄-alkylene-SO₂NR_{B}⁶R_{B}⁷, C₀-C₄-alkylene-CONR_{B}⁶R_{B}⁷, C₁-C₄-alkylene-OCONR_{B}⁶R_{B}⁷, C₁-C₄-alkylene-SOR_{B}⁵, C₁-C₄-alkylene-SO₂R_{B}⁵, NHCOO—C₀-C₄-alkylene-(C₆-C₁₀)-aryl, NHCOO—(C₆-C₁₀)-aryl, NH₂, NH(C₁-C₄-alkyl), N(C₁-C₄-alkyl)₂, morpholin-4-yl, pyrrolidin-1-yl, piperidin-1-yl, 4-piperazin-1-yl, 4-(C₁-C₄-alkyl)piperazin-1-yl;
where two of the radicals R_{B}¹, R_{B}², R_{B}³, or R_{B}⁴ in adjacent (“ortho”) position to one another may also form a fused, unsaturated or aromatic 3- to 10-membered carbocycle which is optionally substituted one or more times identically or differently by the radicals C₁-C₆-alkyl-, OCH₃ or halogen,
in which
R_{B}⁵ is independently of its respective occurrence hydrogen, a branched or unbranched, optionally substituted C₁-C₆-alkyl, C₂-C₆-alkenyl, C₂-C₆-alkynyl, C₁-C₅-alkylene-C₁-C₄-alkoxy, mono- or bis-(C₁-C₆)-alkylamino-(C₁-C₄)-alkylene or (C₁-C₆)-acylamino-(C₁-C₄)-alkylene radical or an optionally substituted (C₆-C₁₀)-aryl-, C₃-C₇-heterocycloalkyl-, C₃-C₇-heterocycloalkenyl-, (C₃-C₁₀)-hetaryl, C₃-C₇-cycloalkyl-, C₁-C₄-alkylene-C₃-C₇-cycloalkyl-, C₁-C₄-alkylene-(C₆-C₁₀)-aryl, C₁-C₄-alkylene-C₃-C₇-heterocycloalkyl-, C₁-C₄-alkylene-C₃-C₇-heterocycloalkenyl- or C₁-C₄-alkylene-(C₂-C₁₀)-hetaryl,
R_{B}⁶ and R_{B}⁷ are independently of one another and independently of their respective occurrence hydrogen, a branched or unbranched, optionally substituted C₁-C₆-alkyl, C₂-C₆-alkenyl, C₂-C₆-alkynyl, C₁-C₆-alkylene-C₁-C₄-alkoxy, mono- or bis-(C₁-C₆)-alkylamino-(C₁-C₄)-alkylene or (C₁-C₆)-acylamino-(C₁-C₄)-alkylene radical or an optionally substituted (C₆-C₁₀)-aryl, C₃-C₇-heterocycloalkyl, C₃-C₇-heterocycloalkenyl, (C₂-C₁₀)-hetaryl, C₃-C₇-cycloalkyl, C₁-C₄-alkylene-C₃-C₇-cycloalkyl, C₁-C₄-alkylene-(C₆-C₁₀)-aryl, C₁-C₄-alkylene-C₃-C₇-heterocyloalkyl, C₁-C₄-alkylene-C₃-C₇-heterocyloalkenyl or C₁-C₄-alkylene-(C₂-C₁₀)-hetaryl radical, or a radical —SO₂R_{B}⁵, —CO₂R_{B}⁵, —CO—NR_{B}⁵R_{B}⁵, or COR_{B}⁵; or R_{B}⁶ and R_{B}⁷ are independently of their respective occurrence together a 3- to 7-membered, optionally substituted, or preferably C₁-C₆-alkyl-, OMe, halogen-substituted, saturated, unsaturated or aromatic heterocycle which, in addition to the ring nitrogen atom, may comprise up to three further different or identical heteroatoms selected from the group consisting of O, N and S, and optionally two radicals R^{x} and R^{x} substituted on this heterocycle together are a fused, saturated, unsaturated or aromatic carbocycle or heterocycle which may comprise up to three different or identical heteroatoms selected from the group consisting of O, N and S, and the ring may optionally be substituted or a further, optionally substituted ring may be fused to this ring,
X is one of the radicals hydrogen, Br, F, Cl, I, C₁-C₄-alkylene-CN, CN, C₁-C₆-haloalkyl, C₁-C₆-haloalkoxy, NO₂, C₁-C₄-alkylene-OR_{X}¹, OR_{X}¹, O—C₁-C₄-alkylene-(C₆-C₁₀-aryl, O—(C₆-C₁₀)-aryl, O—C₁-C₄-alkylene-hetaryl, O-hetaryl, C₁-C₄-alkylene-(C₆-C₁₀)-aryl, (C₆-C₁₀)-aryl, C₁-C₄-alkylene-hetaryl, (C₂-C₁₀)-hetaryl, C₁-C₆-alkyl, C₂-C₆-alkenyl, C₂-C₆-alkynyl, C₂-C₆-alkenyloxy, C₂-C₆-alkynyloxy, C₁-C₄-alkylthio, C₁-C₄-alkylene-NR_{X}²R_{X}³, NR_{X}²R_{X}³, C₁-C₄-alkylene-COR_{X}¹, CORₓ¹, SO₂R_{X}¹, C₁-C₄-alkylene-COOR_{X}¹, COOR_{X}¹, O—C₁-C₄-alkylene-COOR_{X}¹, C₁-C₄-alkylene-SR_{X}¹, SR_{X}¹, C₁-C₄-alkylene-C₃-C₇-cycloalkyl, C₃-C₇-cycloalkyl, C₁-C₄-alkylene-C₃-C₇-heterocycloalkyl, C₃-C₇-heterocycloalkyl, C₁-C₄-alkylene-C₃-C₇-heterocycloalkenyl, C₃-C₇-heterocycloalkenyl, C₁-C₄-alkylene-OCOR_{X}¹, OCOR_{X}¹, C₁-C₄-alkylene-SO₂NR_{X}²R_{X}³, SO₂NR_{X}²R_{X}³, C₁-C₄-alkylene-CONR_{X}²R_{X}³, CONR_{X}²R_{X}³, C₁-C₄-alkylene-OCONR_{X}²R_{X}³, C₁-C₄-alkylene-SOR_{X}¹, C₁-C₄-alkylene-SO₂R_{X}¹, NHCOO—C₀-C₄-alkylene-(C₆-C₁₀-aryl or NHCOO—(C₆-C₁₀)-aryl,
in which
R_{X}¹ is independently of its respective occurrence hydrogen, a branched or unbranched, optionally substituted radical C₁-C₆-alkyl-, C₂-C₆-alkenyl-, C₂-C₆-alkynyl, C₁-C₅-alkylene-C₁-C₄-alkoxy-, mono- or bis-(C₁-C₆)-alkylamino-(C₁-C₄)-alkylene- or (C₁-C₆)-acylamino-(C₁-C₄)-alkylene or an optionally substituted (C₆-C₁₀-aryl-, C₃-C₇-heterocycloalkyl-, C₃-C₇-heterocycloalkenyl-, (C₂-C₁₀)-hetaryl, C₃-C₇-cycloalkyl-, C₁-C₄-alkylene-C₃-C₇-cycloalkyl-, C₁-C₄-alkylene-(C₆-C₁₀-aryl, C₁-C₄-alkylene-C₃-C₇-heterocyloalkyl, C₁-C₄-alkylene, C₃-C₇-heterocyloalkenyl or C₁-C₄-alkylene-(C₂-C₁₀-hetaryl,
R_{X}² and R_{X}³ are independently of one another and independently of their respective occurrence hydrogen, a branched or unbranched, optionally substituted radical C₁-C₆-alkyl-, C₂-C₆-alkenyl-, C₂-C₆-alkynyl, C₁-C₅-alkylene-C₁-C₄-alkoxy-, mono- or bis-(C₁-C₆)-alkylamino-(C₁-C₄)-alkylene- or (C₁-C₆)-acylamino-(C₁-C₄)-alkylene or an optionally substituted radical (C₆-C₁₀)-aryl-, C₃-C₇heterocycloalkyl-, C₃-C₇-heterocycloalkenyl-, (C₂-C₁₀)-hetaryl, C₃-C₇-cycloalkyl-, C₁-C₄-alkylene-C₃-C₇cycloalkyl-, C₁-C₄-alkylene-(C₆-C₁₀)-aryl, C₁-C₄-alkylene-C₃-C₇heterocyloalkyl-, C₁-C₄-alkylene-C₃-C₇-heterocyloalkenyl- or C₁-C₄-alkylene-(C₂-C₁₀)-hetaryl, or a radical —SO₂R_{X}¹, —CO₂R_{X}¹, —CO—NR_{X}¹R_{X}¹, or COR_{X}¹,
or R_{X}² and R_{X}³ together are a 3, 4, 5, 6 or 7 membered, optionally substituted, optionally preferably substituted by C₁-C₆-alkyl-, OCH₃, and/or halogen, saturated, unsaturated or aromatic (C₂-C₁₀)-heterocycle which, in addition to the ring nitrogen atom, may comprise one, two or three further different or identical heteroatoms selected from the group consisting of O, N, and S, and optionally two radicals R_{X}⁴ and R_{X}⁵ substituted on this heterocycle are together a mono- or fused bi- or tricycle having a total of 3 to 21 ring atoms, which may in each case be saturated, unsaturated or aromatic and optionally be substituted by up to six radicals selected from the group consisting of C₁-C₆-alkyl-, OCH₃ and halogen, where at least one ring may comprise a ring nitrogen atom, and additionally up to three further different or identical heteroatoms selected from the group consisting of O, N and S may be present in each ring independently of one another;
Y is a radical 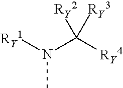
in which
R_{Y}¹ is H, C₁-C₆-alkyl or C₃-C₇-cycloalkyl,
R_{Y}² is H, C₁-C₆-alkyl or C₃-C₇-cycloalkyl,
in which R_{Y}¹ and R_{Y}² may also together form a 4-, 5-, 6- or 7-membered, saturated or unsaturated ring which may include a heteroatom selected from the group consisting of O, S and NR_{Y}⁵ as ring member, where R_{Y}⁵ may be independently of its respective occurrence hydrogen, C₁-C₄-alkyl or C₃-C₇-cycloalkyl, and where the ring may include one or two substituents R_{Y}⁶ and R_{Y}⁷ which are selected independently of one another and independently of their respective occurrence from the group consisting of the radicals C₁-C₆-alkyl, CN, OR_{Y}⁸, NR_{Y}⁹R_{Y}¹⁰, CONR_{Y}⁹R_{Y}¹⁰ and halogen;
or
R_{Y}⁶ and R_{Y}⁷ may also form independently of their respective occurrence together with the C atoms to which they are bonded a fused phenyl ring or a fused 5- or 6-membered aromatic heterocycle which includes 1, 2, 3 or 4 heteroatoms which are selected from the group consisting of N, O and S, where the fused phenyl ring and/or the fused aromatic heterocycle may include independently of one another one, two or three substituents R_{Y}¹⁴ which are selected independently of one another and independently of their respective occurrence from the group consisting of the radicals C₁-C₆-alkyl, CN, OR_{Y}⁸, NR_{Y}⁹R_{Y}¹⁰ NO₂, SR_{Y}¹¹, SO₂R_{Y}¹¹, SO₂NR_{Y}⁹R_{Y}¹⁰, CONR_{Y}⁹R_{Y}¹⁰, COOR_{Y}¹², COR_{Y}¹³, C₁-C₄-haloalkoxy, C₂-C₆-alkenyl, C₂-C₆-alkynyl, C₂-C₆-alkenyloxy, C₂-C₆-alkynyloxy, C₃-C₇-cycloalkyl, C₃-C₇-cycloalkyloxy and halogen;
in which
R_{Y}⁸, R_{Y}⁹, R_{Y}¹⁰, R_{Y}¹¹, R_{Y}¹² and R_{Y}¹³ are selected independently of one another and independently of their respective occurrence from the group consisting of H, optionally substituted C₁-C₆-alkyl, optionally substituted C₃-C₇-cycloalkyl and optionally substituted phenyl,
where R_{Y}⁸ may also independently of its respective occurrence be a radical —(CH₂)ₙ—COR_{Y}¹⁵ or —CO—(CH₂)ₙ—CONR_{Y}¹⁶R_{Y}¹⁷,
in which
R_{Y}¹⁵ is independently of its respective occurrence H, OH, C₁-C₆-alkyl, C₁-C₆-alkoxy, C₃-C₇-cycloalkyl, CH₂CH₂COOH, NR_{Y}¹⁸R_{Y}¹⁹, preferably H, CH₃, C₂H₅, isopropyl, cyclohexyl, —CH₂CH₂COOH, NH₂, N(CH₃)₂;
R_{Y}¹⁶ and R_{Y}¹⁷ are independently of one another and their respective occurrence, selected from the group consisting of H, C₁-C₆-alkyl and C₃-C₆-cycloalkyl;
or R_{Y}¹⁶ and R_{Y}¹⁷ independently of their respective occurrence may together form a ring selected from the group consisting of azetidin-1-yl, pyrrolidin-1-yl, piperidin-1-yl, piperazin-1-yl, morpholin-4-yl and thiomorpholin-4-yl;
R_{Y}¹⁸ is independently of its respective occurrence H, C₁-C₆-alkyl, or C₃-C₆-cycloalkyl;
R_{Y}¹⁹ is independently of its respective occurrence H, C₁-C₆-alkyl, C₃-C₆-cycloalkyl, —C(CH₃)₂CH₂OH, —C(CH₃)(CH₂OH)₂, or —C(CH₂OH)₃;
or R_{Y}¹⁸ and R_{Y}¹⁹ independently of their respective occurrence may together form a ring selected from the group consisting of azetidin-1-yl, pyrrolidin-1-yl, piperidin-1-yl, piperazin-1-yl, morpholin-4-yl and thiomorpholin-4-yl;
R_{Y}¹⁰ may also independently of its respective occurrence be a group COR_{Y}²⁰ in which R_{Y}²⁰ is independently of its respective occurrence hydrogen, optionally substituted C₁-C₄-alkyl or optionally substituted phenyl,
or where R_{Y}⁹ may also form with R_{Y}¹⁰ irrespective of their respective occurrence together a 5- or 6-membered, saturated or unsaturated carbocycle which may include a heteroatom selected from the group consisting of O, S, and NR_{Y}¹⁴, as ring member, where R_{Y}¹⁴ is hydrogen or C₁-C₄-alkyl,
n is independently of its respective occurrence the integer 1 or 2;
R_{Y}⁴ is independently of its respective occurrence H, C₁-C₆-alkyl or C₃-C₆-cycloalkyl, preferably H;
R_{Y}⁴ is independently of its respective occurrence H, CONR_{Y}²¹R_{Y}²², CR_{Y}²³R_{Y}²⁴NR_{Y}²¹ R_{Y}²², COOH or COO—C₁-C₄-alkyl;
R_{Y}²¹, R_{Y}²², R_{Y}²³ and R_{Y}²⁴ are independently of one another and independently of their respective occurrence H, C₁-C₆-alkyl or C₃-C₆-cycloalkyl;
or R_{Y}²¹ and R_{Y}²² may also independently of their respective occurrence together form a 4-, 5- or 6-membered, saturated or unsaturated carbocycle which may include a heteroatom selected from the group consisting of O, S, and NR_{Y}²⁵, as ring member, where R_{Y}²⁵ is independently of its respective occurrence hydrogen or C₁-C₄-alkyl;
the tautomeric, enantiomeric and diastereomeric forms thereof, and the prodrugs thereof, and the physiologically tolerated salts of said compound.

2. A compound as claimed in claim 1, in which
A is C₆-C₁₀-aryl which may be substituted by one, two, three or four radicals selected from the group consisting of R_{A}¹, R_{A}², R_{A}³ and R_{A}⁴, in which R_{A}¹, R_{A}², R_{A}³ and R_{A}⁴ are selected independently of one another and independently of their respective occurrence from the group consisting of hydrogen, chlorine, bromine, iodine, fluorine, CN, OR_{A}⁵, COR_{A}⁵, COOR_{A}⁵, SR_{A}⁵, C₃-C₇-cycloalkyl, OCOR_{A}⁵, SO₂NR_{A}⁶R_{A}⁷, CONR_{A}⁶R_{A}⁷, C₀-C₄-alkylene-CN, C₁-C₆-haloalkyl, C₁-C₆-haloalkoxy, NO₂, C₀-C₄-alkylene-OR_{A}⁵, C₁-C₆-alkyl, C₂-C₆-alkenyl, C₂-C₆-alkynyl, C₂-C₆-alkenyloxy, C₂-C₆-alkynyloxy, C₁-C₄-alkylthio, C₀-C₄-alkylene-COR_{A}⁵, SO₂R_{A}⁵, C₀-C₄-alkylene-COOR_{A}⁵, O—C₁-C₄-alkylene-COOR_{A}⁵, C₀-C₄-alkylene-SR_{A}⁵, C₀-C₄-alkylene-C₃-C₇-cycloalkyl, C₀-C₄-alkylene-OCOR_{A}⁵, C₀-C₄-alkylene-SO₂NR_{A}⁶R_{A}⁷, C₀-C₄-alkylene-CONR_{A}⁶R_{A}⁷, C₁-C₄-alkylene-OCONR_{A}⁶R_{A}⁷, C₁-C₄-alkylene-SOR_{A}⁵, C₁-C₄-alkylene-SO₂R_{A}⁵, NHCOO—C₀-C₄-alkylene-aryl and NHCOOH,
where two of the radicals R_{A}¹, R_{A}², R_{A}³ and R_{A}⁴ in adjacent position (“ortho”) to one another may also form an optionally substituted, fused saturated, unsaturated or aromatic 3- to 10-membered carbocycle or a cyclic acetal —O—CH₂—CH₂—O— or —O—CH₂—O—,
and in which
R_{A}⁵ is independently of its respective occurrence hydrogen, a branched or unbranched radical C₁-C₆-alkyl, or a branched or unbranched, optionally substituted radical C₂-C₆-alkenyl-, C₂-C₆-alkynyl, C₃-C₇-cycloalkyl-, C₁-C₄-alkylene-C₃-C₇-cycloalkyl- or C₁-C₄-alkylene-(C₆-C₁₀)-aryl,
R_{A}⁶ and R_{A}⁷ are independently of one another and independently of their respective occurrence hydrogen, a branched or unbranched, optionally substituted radical C₁-C₆-alkyl-, C₂-C₆-alkenyl-, C₂-C₆-alkynyl, C₁-C₅-alkylene-C₁-C₄-alkoxy-, C₃-C₇-cycloalkyl-, C₁-C₄-alkylene-C₃-C₇-cycloalkyl, C₁-C₄-alkylene-aryl, or a radical —SO₂R_{A}⁵, —CO₂R_{A}⁵, —CO—NR_{A}⁵ R_{A}⁵, or —COR_{A}⁵, and
B is an aromatic or partly aromatic C₆-C₁₀-mono- or fused bicycle which may be substituted by a maximum of four radicals selected from the group consisting of R_{B}¹, R_{B}², R_{B}³, and R_{B}⁴, where R_{B}¹, R_{B}², R_{B}³ and R_{B}⁴ are selected independently of one another and independently of their respective occurrence from the group consisting of hydrogen, chlorine, bromine, iodine, fluorine, CN, OR_{B}⁵, COR_{B}⁵, COOR_{B}⁵, SR_{B}⁵, C₃-C₇-cycloalkyl, OCOR_{A}⁵, SO₂NR_{A}⁶R_{A}⁷, CONR_{A}⁶R_{A}⁷, (C₆-C₁₀)-aryl, (C₃-C₁₀)-hetaryl, NR_{B}⁶R_{B}⁷, C₃-C₇-heterocycloalkyl, C₃-C₇-heterocycloalkenyl, OCOR_{B}⁵, SO₂NR_{B}⁶R_{B}⁷, CONR_{B}⁶R_{B}⁷, C₀-C₄-alkylene-CN, C₁-C₆-haloalkyl, C₁-C₆-haloalkoxy, NO₂, C₀-C₄-alkylene-OR_{B}⁵, O—C₀-C₄-alkylene-(C₆-C₁₀)-aryl, O—C₀-C₄-alkylene-(C₂-C₁₀)-hetaryl, C₀-C₄-alkylene-(C₆-C₁₀)-aryl, C₀-C₄-alkylene-(C₂-C₁₀)-hetaryl, C₁-C₄-alkyl, C₂-C₆-alkenyl, C₂-C₆-alkynyl, C₂-C₆-alkenyloxy, C₂-C₆-alkynyloxy, C₁-C₄-alkylthio, C₀-C₄-alkylene-NR_{B}⁶R_{B}⁷, C₀-C₄-alkylene-COR_{B}⁵, SO₂R_{B}⁵, C₀-C₄-alkylene-COOR_{B}⁵, O—C₁-C₄-alkylene-COOR_{B}⁵, C₀-C₄-alkylene-SR_{B}⁵, C₀-C₄-alkylene-C₃-C₇-cycloalkyl, C₀-C₄-alkylene-C₃-C₇-heterocycloalkyl, C₀-C₄-alkylene-C₃-C₇-heterocycloalkenyl, C₀-C₄-alkylene-OCOR_{B}⁵, C₀-C₄-alkylene-SO₂NR_{B}⁶R_{B}⁷, C₀-C₄-alkylene-CONR_{B}⁶R_{B}⁷, C₁-C₄-alkylene-OCONR_{B}⁶R_{B}⁷, C₁-C₄-alkylene-SOR_{B}⁵, C₁-C₄-alkylene-SO₂R_{B}⁵, NHCOO—C₀-C₄-alkylene-(C₆-C₁₀)-aryl and NHCOO—(C₆-C₁₀)-aryl,
where two of the radicals R_{B}¹, R_{B}², R_{B}³, or R_{B}⁴ in adjacent (“ortho”) position to one another may also form a fused, unsaturated or aromatic 3- to 10-membered carbocycle which is optionally substituted one or more times identically or differently by the radicals C₁-C₆-alkyl-, OCH₃ or halogen,
in which
R_{B}⁵ is independently of its respective occurrence hydrogen, a branched or unbranched, optionally substituted C₁-C₆-alkyl, C₂-C₆-alkenyl, C₂-C₆-alkynyl, C₁-C₆-alkylene-C₁-C₄-alkoxy, mono- or bis-(C₁-C₆)-alkylamino-(C₁-C₄)-alkylene or (C₁-C₆)-acylamino-(C₁-C₄)-alkylene radical or an optionally substituted (C₆-C₁₀)-aryl-, C₃-C₇-heterocycloalkyl-, C₃-C₇-heterocycloalkenyl-, (C₂-C₁₀)-hetaryl, C₃-C₇-cycloalkyl-, C₁-C₄-alkylene-C₃-C₇-cycloalkyl-, C₁-C₄-alkylene-(C₆-C₁₀)-aryl, C₁-C₄-alkylene-C₃-C₇-heterocyloalkyl-, C₁-C₄-alkylene-C₃-C₇-heterocyloalkenyl- or C₁-C₄-alkylene-(C₂-C₁₀)-hetaryl,
R_{B}⁶ and R_{B}⁷ are independently of one another and independently of their respective occurrence hydrogen, a branched or unbranched, optionally substituted C₁-C₆-alkyl-, C₂-C₆-alkenyl-, C₂-C₆-alkynyl, C₁-C₆-alkylene-C₁-C₄-alkoxy-, mono- or bis(C₁-C₆)-alkylamino-(C₁-C₄)-alkylene or (C₁-C₆)-acylamino-(C₁-C₄)-alkylene radical or an optionally substituted (C₆-C₁₀)-aryl-, C₃-C₇-heterocycloalkyl-, C₃-C₇-heterocycloalkenyl-, (C₂-C₁₀)-hetaryl, C₃-C₇-cycloalkyl-, C₁-C₄-alkylene-C₃-C₇-cycloalkyl-, C₁-C₄-alkylene-(C₆-C₁₀)-aryl, C₁-C₄-alkylene-C₃-C₇-heterocycloalkyl-, C₁-C₄-alkylene-C₃-C₇-heterocycloalkenyl- or C₁-C₄-alkylene-(C₂-C₁₀)-hetaryl, or a radical —SO₂R_{B}⁵, —CO₂R_{B}⁵, —CO—NR_{B}⁵R_{B}⁵, or COR_{B}⁵;
or R_{B}⁶ and R_{B}⁷ are independently of their respective occurrence together a 3 to 7 membered, optionally substituted, or preferably C₁-C₆-alkyl-, Ome-, halogen-substituted, saturated, unsaturated or aromatic heterocycle which, in addition to the ring nitrogen atom, may comprise up to three further different or identical heteroatoms selected from the group consisting of O, N and S, and optionally two radicals R^{x} and R^{x} substituted on this heterocycle are together a fused, saturated, unsaturated or aromatic carbocycle or heterocycle which may comprise up to three different or identical heteroatoms selected from the group consisting of O, N and S, and the ring may optionally be substituted, or a further, optionally substituted ring may be fused to this ring,
and in which the variables X and Y have the meaning indicated in claim 1.

3. A compound as claimed in claim 1, in which
A is C₆-C₁₀-aryl which may be substituted by one, two, three or four radicals selected from the group consisting of R_{A}¹, R_{A}², R_{A}³ and/or R_{A}⁴, in which R_{A}¹, R_{A}², R_{A}³ and R_{A}⁴ are selected independently of one another and independently of their respective occurrence from the group consisting of hydrogen, chlorine, bromine, iodine, fluorine, CN, OR_{A}⁵, SR_{A}⁵, C₃-C₇-cycloalkyl, NO₂, C₁-C₆-alkyl, C₂-C₆-alkenyl, C₂-C₆-alkynyl, SO₂R_{A}⁵, C₁-C₄-alkylene-C₃-C₇-cycloalkyl, O—CF₃, CF₃, OCHF₂, NH₂, NH(C₁-C₄-alkyl), N(C₁-C₄-alkyl)₂,
where two of the radicals R_{A}¹, R_{A}², R_{A}³ and R_{A}⁴ in adjacent position (“ortho”) to one another may also form an optionally substituted, fused saturated, unsaturated and/or aromatic 3- to 10-membered carbocycle or a cyclic acetal —O—CH₂—CH₂—O— or —O—CH₂—O—,
and in which
R_{A}⁵ is independently of its respective occurrence hydrogen, a branched or unbranched radical C₁-C₆-alkyl, or a branched or unbranched, optionally substituted radical C₂-C₆-alkenyl-, C₂-C₆-alkynyl, C₃-C₇-cycloalkyl-, C₁-C₄-alkylene-C₃-C₇-cycloalkyl- or C₁-C₄-alkylene-(C₆-C₁₀)-aryl,
B is an aromatic or partly aromatic C₆-C₁₀-mono- or fused -bicycle which may be substituted by one, two or three radicals selected from the group consisting of R_{B}¹, R_{B}² and/or R_{B}³, where R_{B}¹, R_{B}² and R_{B}³ are selected independently of one another and independently of their respective occurrence from the group consisting of hydrogen, chlorine, bromine, iodine, fluorine, CN, CF₃, OCF₃, NO₂, OH, O—C₁-C₄-alkyl, O-phenyl, O—C₁-C₄-alkylene-phenyl, phenyl, C₁-C₆-alkyl, C₂-C₆-alkenyl, C₂-C₆-alkynyl, NH₂, NH(C₁-C₄-alkyl), N(C₁-C₄-alkyl)₂, morpholin-4-yl, pyrrolidin-1-yl, piperidin-1-yl, 4-piperazin-1-yl, 4-(C₁-C₄-alkyl)-piperazin-1-yl;
and in which the variables X and Y have the meaning indicated in claim 1.

4. A compound as claimed in claim 1, in which
A is a phenyl ring which may be substituted by one, two, three or four radicals selected from the group consisting of R_{A}¹, R_{A}², R_{A}³ and R_{A}⁴, where R_{A}¹, R_{A}², R_{A}³ and R_{A}⁴ have the meanings stated in claim 1;
B is a phenyl ring which may be substituted by one, two, three or four radicals selected from the group consisting of R_{B}¹, R_{B}², R_{B}³ and R_{B}⁴, R_{B}¹, R_{B}², R_{B}³ and R_{B}⁴ have the meaning stated in claim 1;
X is hydrogen, F, Cl, CF₃, OCF₃, O—C₁-C₄-alkyl, OH, C₁-C₆-alkyl or C₃-C₆-cycloalkyl; and
Y has the meaning stated in claim 1,
the tautomeric, enantiomeric and diastereomeric forms thereof, and the prodrugs thereof, and the physiologically tolerated salts of said compound.

5. A compound as claimed in claim 1, in which
A is a phenyl ring which may be substituted by one or two radicals selected from the group consisting of R_{A}¹ and R_{A}² which are selected independently of one another from the group consisting of hydrogen, chlorine, fluorine, O—C₁-C₄-alkyl, OH, (CH₂)₁₋₂—O—(CH₂)₁₋₂—CH₃, O—(CH₂)₁₋₂—CH₃, OCH₃, (CH₂)₁₋₂—OCH₃, C₁-C₆-alkyl, C₃-C₇cycloalkyl, O—C₃-C₆-cycloalkyl, CN, CF₃, OCF₃,
where R_{A}¹ and R_{A}² may also in adjacent position (“ortho”) together form a cyclic acetal —O—CH₂—O—;
B is a phenyl ring which may be substituted by one, two, three or four radicals selected from the group consisting of R_{B}¹, R_{B}², R_{B}³ and R_{B}⁴, where R_{B}¹, R_{B}², R_{B}³ and R_{B}⁴ are selected independently of one another and independently of their respective occurrence from the group consisting of hydrogen, fluorine, chlorine, CF₃, OCF₃, OCHF₂, CN, O—C₁-C₄-alkyl, OH, C₁-C₆-alkyl and C₃-C₇-cycloalkyl-,
or where two adjacent radicals R_{B}¹, R_{B}², R_{B}³ or R_{B}⁴ in adjacent position (“ortho”) may together form an optionally substituted, fused, saturated and/or aromatic 3- to 10-membered carbocycle;
X is hydrogen;
Y is a radical 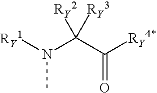
in which
R_{Y}¹ is H, C₁-C₆-alkyl or C₃-C₆-cycloalkyl,
R_{Y}² is H, C₁-C₆-alkyl or C₃-C₆-cycloalkyl,
R_{Y}³ is H, C₁-C₆-alkyl or C₃-C₆-cycloalkyl,
R_{Y}^{4*} is NR_{Y}¹³R_{Y}¹⁴ or O—C₁-C₄-alkyl in which
R_{Y}¹³ is H, C₁-C₆-alkyl or C₃-C₆-cycloalkyl,
R_{Y}¹⁴ is H, C₁-C₆-alkyl or C₃-C₆-cycloalkyl,
where R_{Y}¹³ and R_{Y}¹⁴ may also together form a 4-, 5- or 6-membered, saturated or unsaturated ring which may include a heteroatom selected from the group consisting of O, S and NR_{Y}¹⁷ as ring member, where R_{Y}¹⁷ is hydrogen, C₁-C₄-alkyl or C₃-C₇-cycloalkyl-,
Or Y is a radical 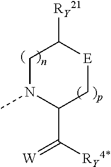
in which
n is 0, 1 or 2,
p is 0, 1 or 2,
with the proviso that the total of n and p is 1 or 2,
E is O, S, NR_{Y}²³ or C(C₁-C₄-alkyl)R_{Y}²², CHR_{Y}²²
W is O or S,
R_{Y}^{4*} is NR_{Y}¹³R_{Y}¹⁴ or O—C₁-C₄-alkyl,
R_{Y}¹³ is H, C₁-C₆-alkyl or C₁-C₆-cycloalkyl,
R_{Y}¹⁴ is H, C₁-C₆-alkyl or C₁-C₆-cycloalkyl,
where R_{Y}¹³ and R_{Y}¹⁴ may also together form a 4-, 5- or 6-membered, saturated or unsaturated ring which may include a heteroatom selected from the group consisting of O, S and NR_{Y}¹⁷ as ring member, where R_{Y}¹⁷ is hydrogen, C₁-C₄-alkyl or C₃-C₆-cycloalkyl.
R_{Y}²¹ is selected from the group consisting of the radicals optionally substituted C₁-C₆-alkyl, optionally substituted C₃-C₆-cycloalkyl, CN, OR_{Y}⁸, NR_{Y}⁹R_{Y}¹⁰, CONR_{Y}⁹R_{Y}¹⁰ and halogen; in which
R_{Y}⁸, R_{Y}⁹, R_{Y}¹⁰, R_{Y}¹¹, R_{Y}¹² and R_{Y}¹³ are independently of one another and independently of their respective occurrence H, optionally substituted C₁-C₆-alkyl, optionally substituted C₃-C₆-cycloalkyl or optionally substituted phenyl,
where
R_{Y}⁸ may also independently of its respective occurrence and independently of its respective occurrence be a radical —(CH₂)_{q}—COR_{Y}¹⁵ or —CO—(CH₂)q—CONR_{Y}¹⁶R_{Y}¹⁷;
in which
R_{Y}¹⁵ is independently of its respective occurrence H, OH, C₁-C₆-alkyl, C₁-C₆-alkoxy, C₃-C₇-cycloalkyl, CH₂CH₂COOH, NR_{Y}¹⁸R_{Y}¹⁹, preferably H, CH₃, C₂H₅, iso-C₃H₇, cyclohexyl, —CH₂CH₂COOH, NH₂ or N(CH₃)₂;
R_{Y}¹⁶ and R_{Y}¹⁷ are independently of one another and independently of their respective occurrence H, C₁-C₆-alkyl or C₃-C₆-cycloalkyl, or R_{Y}¹⁶ and R_{Y}¹⁷ may independently of their respective occurrence together also be a ring selected from the group consisting of azetidin-1-yl, pyrrolidin-1-yl, piperidin-1-yl, piperazin-1-yl, morpholin-4-yl and thiomorpholin-4-yl;
R_{Y}¹⁸ is independently of its respective occurrence H, C₁-C₆-alkyl or C₃-C₆-cycloalkyl;
R_{Y}¹⁹ is independently of its respective occurrence H, C₁-C₆-alkyl, C₃-C₆-cycloalkyl, —C(CH₃)₂CH₂OH, —C(CH₃)(CH₂OH)₂, —C(CH₂OH)₃,
or R_{Y}¹⁸ and R_{Y}¹⁹ may independently of their respective occurrence together form a ring selected from the group consisting of azetidin-1-yl, pyrrolidin-1-yl, piperidin-1-yl, piperazin-1-yl, morpholin-4-yl and thiomorpholin-4-yl;
R_{Y}¹⁰ may independently of its respective occurrence also be a group COR_{Y}²⁰, where R_{Y}²⁰ is hydrogen, optionally substituted C₁-C₄-alkyl or optionally substituted phenyl,
or R_{Y}⁹ and R_{Y}¹⁰ may also independently of their respective occurrence together form a 5- or 6-membered, saturated or unsaturated carbocycle which may include a heteroatom selected from the group consisting of O, S and NR_{Y}¹⁴ as ring member, where R_{Y}¹⁴ is hydrogen or C₁-C₄-alkyl,
where
q is independently of its respective occurrence 1 or 2,
R_{Y}²² is H or C₁-C₄-alkyl,
or
R_{Y}²¹ and R_{Y}²² may also independently of their respective occurrence form together with the C atoms to which they are bonded a fused phenyl ring or a fused 5- or 6-membered aromatic heterocycle which includes 1, 2, 3 or 4 heteroatoms which are selected from the group of N, O and S, where the fused phenyl ring and the fused aromatic heterocycle may independently of one another have 1, 2 or 3 substituents which are selected independently of one another from the group consisting of optionally substituted C₁-C₆-alkyl, CN, OR_{Y}⁸, NR_{Y}⁹R_{Y}¹⁰ NO₂, SR_{Y}¹¹, SO₂R_{Y}¹¹, SO₂NR_{Y}⁹R_{Y}¹⁰, CONR_{Y}⁹R_{Y}¹⁰, COOR_{Y}¹², COR_{Y}¹³, C₁-C₄-haloalkoxy, C₂-C₆-alkenyl, C₂-C₆-alkynyl, C₂-C₆-alkenyloxy, C₂-C₆-alkynyloxy, C₃-C₆-cycloalkyl, C₃-C₆-cycloalkyloxy and halogen; in which
R_{Y}²³ is independently of its respective occurrence H, C₁-C₆-alkyl, C₃-C₆-cycloalkyl or COCH₃;
the tautomeric, enantiomeric and diastereomeric forms thereof, and the prodrugs thereof, and the physiologically tolerated salts of said compound.

6. A compound as claimed in claim 1, in which
A is the group 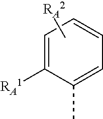
in which
R_{A}¹ and R_{A}² are selected independently of one another from the group consisting of hydrogen, chlorine, fluorine, O—C₁-C₄-alkyl, OH, (CH₂)₁₋₂—O—(CH₂)₁₋₂—CH₃, O—(CH₂)₁₋₂—CH₃, (CH₂)₁₋₂—OCH₃, OCH₃, C₁-C₆-alkyl, C₃-C₇-cycloalkyl, O—C₃-C₇-cycloalkyl, CN, CF₃ and OCF₃,
where R_{A}¹ and R_{A}² in adjacent position (“ortho”) may also together form a cyclic acetal —O—CH₂—O—;
B is a phenyl ring which may be substituted by the radicals R_{B}¹ and R_{B}², where R_{B}¹ and R_{B}² are selected independently of one another from the group consisting of hydrogen, fluorine, chlorine, CN, CF₃, OCF₃, OCHF₂, OH, O—C₁-C₄-alkyl, C₁-C₆-alkyl and C₃-C₇-cycloalkyl,
X is hydrogen,
Y is a radical selected from the group consisting of 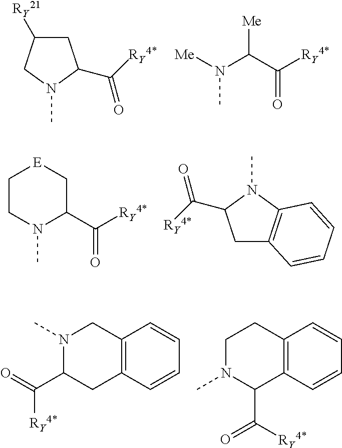
in which R_{Y}²¹ is selected from the group consisting of hydrogen, optionally substituted C₁-C₆-alkyl, optionally substituted C₃-C₇-cycloalkyl, CN, OR_{Y}⁸, NR_{Y}⁹R_{Y}¹⁰, CONR_{Y}⁹R_{Y}¹⁰ and halogen; in which
R_{Y}⁸, R_{Y}⁹ and R_{Y}¹⁰ are independently of one another and independently of their respective occurrence H, optionally substituted C₁-C₆-alkyl, optionally substituted C₃-C₇-cycloalkyl or optionally substituted phenyl,
where
R_{Y}⁸ may also be a radical —(CH₂)ₙ, —COR_{Y}¹⁵ or —CO—(CH₂)ₙ—CONR_{Y}¹⁶R_{Y}¹⁷, in which
R_{Y}¹⁵ is independently of its respective occurrence H, OH, C₁-C₆-alkyl, C₁-C₆-alkoxy, C₃-C₇-cycloalkyl, CH₂CH₂COOH, NR_{Y}¹⁸R_{Y}¹⁹, preferably H, CH₃, C₂H₅, iso-C₃H₇, cyclohexyl, —CH₂CH₂COOH, NH₂, N(CH₃)₂;
R_{Y}¹⁶ and R_{Y}¹⁷ are independently of one another and independently of their respective occurrence H, C₁-C₆-alkyl or C₃-C₆-cycloalkyl,
or R_{Y}¹⁸ and R_{Y}¹⁷ may together form a ring selected from the group consisting of azetidin-1-yl, pyrrolidin-1-yl, piperidin-1-yl, piperazin-1-yl, morpholin-4-yl and thiomorpholin-4-yl;
R_{Y}¹⁸ is independently of its respective occurrence H, C₁-C₆-alkyl or C₃-C₆-cycloalkyl,
R_{Y}¹⁹ is independently of its respective occurrence H, C₁-C₆-alkyl, C₃-C₆-cycloalkyl, —C(CH₃)₂CH₂OH, —C(CH₃)(CH₂OH)₂, or —C(CH₂OH)₃,
or R_{Y}¹⁸ and R_{Y}¹⁹ may independently of their respective occurrence together form a ring selected from the group consisting of azetidin-1-yl, pyrrolidin-1-yl, piperidin-1-yl, piperazin-1-yl, morpholin-4-yl and thiomorpholin-4-yl;
and in which
R_{Y}¹⁰ may also independently of its respective occurrence be a radical COR_{Y}²⁰ in which R_{Y}²⁰ is hydrogen, optionally substituted C₁-C₄-alkyl or optionally substituted phenyl,
where R_{Y}⁹ and R_{Y}¹⁰ may independently of their respective occurrence also together form a 5- or 6-membered, saturated or unsaturated carbocycle which may include a heteroatom selected from the group consisting of O, S and NR_{Y}¹⁴ as ring member, in which R_{Y}¹⁴ is hydrogen or C₁-C₄-alkyl,
n independently of its respective occurrence is the integer 1 or 2,
R_{Y}^{4*} is NHEt, NMe₂ or azetidinyl,
E is O or CH₂,
the tautomeric, enantiomeric and diastereomeric forms thereof, and the prodrugs thereof, and the physiologically tolerated salts of said compound.

7. A compound as claimed in claim 1, in which
A is the group 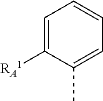
in which
R_{A}¹ is selected from the group consisting of chlorine, methyl, ethyl, OCH₃, OC₂H₅, OC₃H₇, O-i-C₃H₇, fluorine, CF₃, and OCF₃;
B is a phenyl ring which may be substituted by the radicals R_{B}¹ and R_{B}², where R_{B}¹ and R_{B}² are selected independently of one another from the group consisting of hydrogen, fluorine, chlorine, CN, CF₃, OCF₃, OCHF₂, OH, O—C₁-C₄-alkyl, C₃-C₇-cycloalkyl and C₁-C₆-alkyl,
X is hydrogen,
Y is a radical selected from the group consisting of 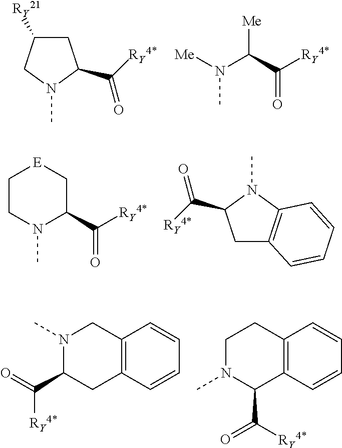
in which R_{Y}²¹ is selected independently of its respective occurrence from the group consisting of hydrogen, optionally substituted C₁-C₆-alkyl, optionally substituted C₃-C₇-cycloalkyl, OR_{Y}⁸, NR_{Y}⁸R_{Y}¹⁰, CONR_{Y}⁹R_{Y}¹⁰ and halogen; in which
R_{Y}⁸, R_{Y}⁹ and R_{Y}¹⁰ are independently of one another and independently of their respective occurrence H, optionally substituted C₁-C₆-alkyl, optionally substituted C₁-C₇-cycloalkyl or optionally substituted phenyl,
where
R_{Y}⁸ may also independently of its respective occurrence be a radical —(CH₂)ₙ—COR_{Y}¹⁵ or —CO—(CH₂)ₙ—CONR_{Y}¹⁶R_{Y}¹⁷,
in which
R_{Y}¹⁵ is independently of its respective occurrence H, OH, C₁-C₆-alkyl, C₁-C₆-alkoxy, C₁-C₆-cycloalkyl, CH₂CH₂COOH, NR_{Y}¹⁸R_{Y}¹⁹, preferably H, CH₃, C₂H₅, iso-C₃H₇, cyclohexyl, —CH₂CH₂COOH, NH₂, or N(CH₃)₂,
R_{Y}¹⁶ and R_{Y}¹⁷ are independently of one another and independently of their respective occurrence H, C₁-C₆-alkyl or C₃-C₇-cycloalkyl,
or R_{Y}¹⁶ and R_{Y}¹⁷ may also independently of their respective occurrence together form a ring selected from the group consisting of azetidin-1-yl, pyrrolidin-1-yl, piperidin-1-yl, piperazin-1-yl, morpholin-4-yl and thiomorpholin-4-yl;
R_{Y}¹⁸ is independently of its respective occurrence H, C₁-C₆-alkyl or C₃-C₆-cycloalkyl,
R_{Y}¹⁹ is independently of its respective occurrence H, C₁-C₆-alkyl, C₃-C₆-cycloalkyl, —C(CH₃)₂CH₂OH, —C(CH₃)(CH₂OH)₂ or —C(CH₂OH)₃;
or R_{Y}¹⁸ and R_{Y}¹⁹ may also independently of their respective occurrence together form a ring selected from the group consisting of azetidin-1-yl, pyrrolidin-1-yl, piperidin-1-yl, piperazin-1-yl, morpholin-4-yl and thiomorpholin-4-yl;
R_{Y}¹⁰ may also independently of its respective occurrence be a group COR_{Y}²⁰ in which R_{Y}²⁰ is hydrogen, optionally substituted C₁-C₄-alkyl or optionally substituted phenyl,
where R_{Y}⁹ and R_{Y}¹⁰ may also independently of their respective occurrence together form a 5- or 6-membered, saturated or unsaturated carbocycle which may include a heteroatom selected from the group consisting of O, S and NR_{Y}¹⁴ as ring member, in which R_{Y}¹⁴ is hydrogen or C₁-C₄-alkyl,
n is independently of its respective occurrence the integer 1 or 2;
R_{Y}^{4*} is independently of its respective occurrence NHC₂H₅, N(CH₃)₂, or azetidinyl,
E is independently of its respective occurrence O or CH₂, in which
the carbon which carries the radical R_{Y}²¹ has the (R) configuration,
the carbon which carries the radical COR_{Y}^{4*} has the (S) configuration, and the carbon in position 3 of the indol-2-one may have the (R) or (S) configuration,
the tautomeric, enantiomeric and diastereomeric forms thereof, and the prodrugs thereof, and the physiologically tolerated salts of said compound.

8. A compound as claimed in claim 1, in which
A is the group 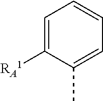
in which R_{A}¹ is selected from the group consisting of chlorine, methyl, ethyl, OCH₃, OC₂H₅, OC₃H₇, O-iso-C₃H₇, fluorine, CF₃ and OCF₃,
B is a phenyl ring which may be substituted by the radicals R_{B}¹ and R_{B}², where R_{B}¹ and R_{B}² are selected independently of one another from the group consisting of hydrogen, fluorine, chlorine, CN, CF₃, OCF₃, OCHF₂, O—C₁-C₄-alkyl, OH, C₁-C₆-alkyl and C₃-C₇-cycloalkyl,
X is hydrogen,
Y is a radical selected from the group consisting of the radicals Y1 to Y20 mentioned below: 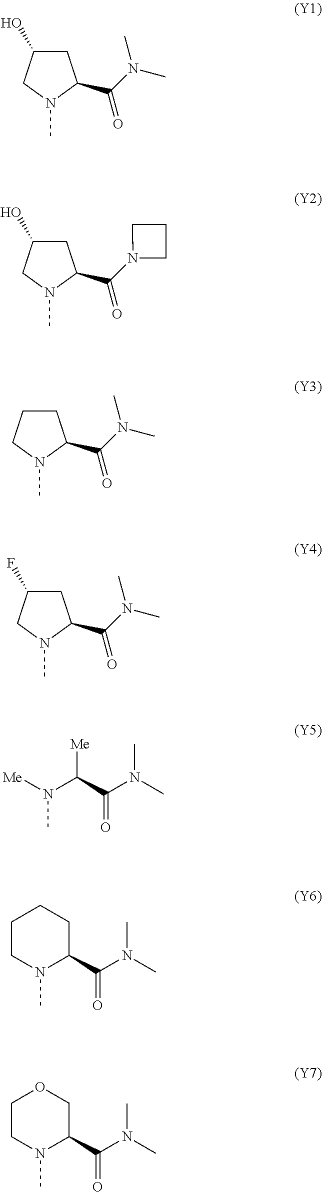 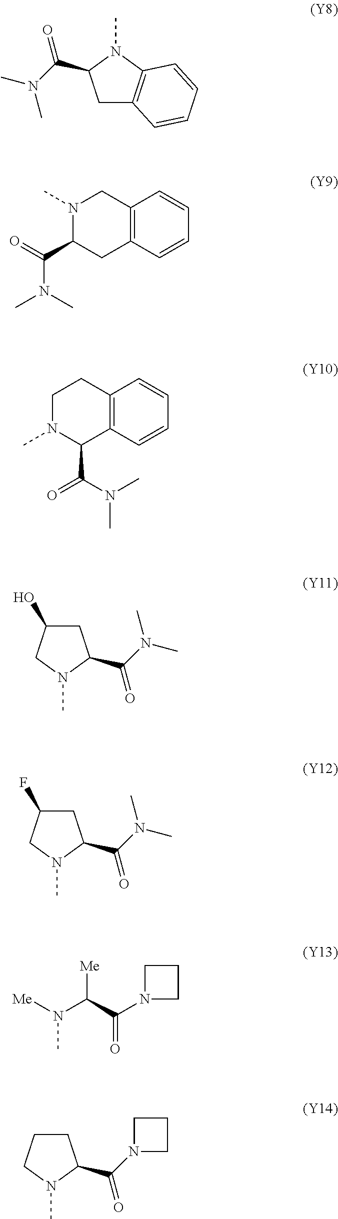 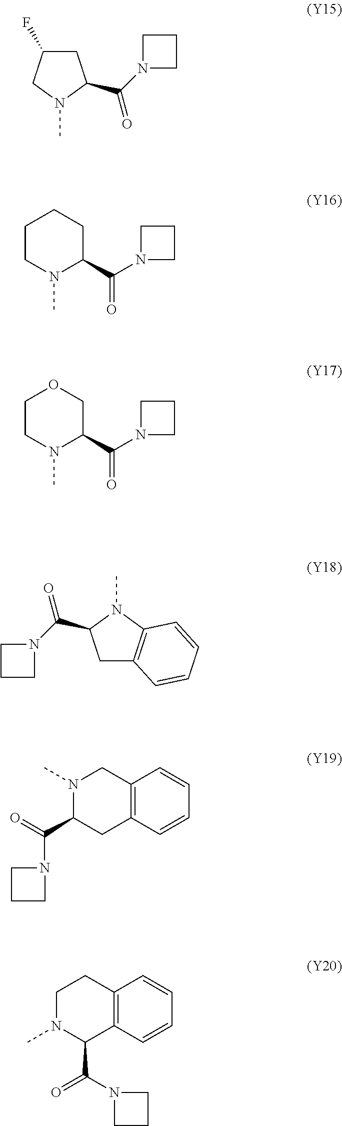
in which
the carbon which carries the radical hydroxy (OH) or fluorine (F) has the (R) or (S) configuration,
the carbon which carries the amide group has the (S) configuration, and the carbon in position 3 of the indol-2-one may have the (R) or (S) configuration,
the tautomeric, enantiomeric and diastereomeric forms thereof, and the prodrugs thereof, and the physiologically tolerated salts of said compound.

9. A compound as claimed in claim 1, characterized in that it rotates the plane of polarized light to the left, that is has a negative rotation.

10. A compound as claimed in claim 1, characterized in that it has a binding affinity Ki for a vasopressin V1b receptor subtype of less than 100 nm.

11. A compound as claimed in claim 1, characterized in that it has a selectivity for the vasopressin V1b receptor subtype vis-à-vis the vasopressin Via receptor subtype, that is the quotient of Ki(V1a)/Ki(V1b) is at least greater than 1.

12. A compound as claimed in claim 1, characterized in that it has a selectivity for the vasopressin V1b receptor subtype vis-à-vis the vasopressin V2 receptor subtype, that is the quotient of Ki(V2)/Ki(V1b) is at least greater than 1.

13. A compound as claimed in claim 1, characterized in that it has a selectivity for the vasopressin V1b receptor subtype vis-à-vis the oxytocin (OT) receptor, that is the quotient of Ki(OT)/Ki(V1b) is at least greater than 1.

14. A compound as claimed in claim 1 any, characterized in that it has a binding affinity Ki for the vasopressin V1b receptor subtype of less than 100 nm and a selectivity for the vasopressin V1b receptor subtype vis-à-vis the vasopressin Via receptor subtype, that is the quotient of Ki(V1a)/Ki(V1b) is at least greater than 1.

15. A compound as claimed in claim 1, characterized in that it has a binding affinity Ki for the vasopressin V1b receptor subtype of less than 100 nm and a selectivity for the vasopressin V1b receptor subtype vis-à-vis the vasopressin V2 receptor subtype, that is the quotient of Ki(V2)/Ki(V1b) is at least greater than 1.

16. A compound as claimed in claim 1, characterized in that it has a binding affinity Ki for the vasopressin V1b receptor subtype of less than 100 nm and a selectivity for the vasopressin V1b receptor subtype vis-à-vis the oxytocin (OT) receptor, that is the quotient of Ki(OT)/Ki(V1b) is at least greater than 1.

17. A compound as claimed in claim 1, characterized in that it has a binding affinity Ki for the vasopressin V1b receptor subtype of less than 100 nm and selectivities for the vasopressin V1b receptor subtype vis-à-vis the vasopressin Via receptor subtype and the vasopressin V2 receptor subtype, that is the quotients of Ki(V1a)/Ki(V1b) and Ki(V2)/Ki(V1b) are in each case at least greater than 1.

18. A compound as claimed in claim 1, characterized in that it has a binding affinity Ki for the vasopressin V1b receptor subtype of less than 100 nm and simultaneous selectivities for the vasopressin V1b receptor subtype vis-à-vis the vasopressin V1a receptor subtype and the oxytocin (OT) receptor, that is the quotients of Ki(V1a)/Ki(V1b) and Ki(OT)/Ki(V1b) are in each case at least greater than 1.

19. A compound as claimed in claim 1, characterized in that it has a binding affinity Ki for the vasopressin V1b receptor subtype of less than 100 nm and simultaneous selectivities for the vasopressin V1b receptor subtype vis-à-vis the vasopressin V2 receptor subtype and the oxytocin (OT) receptor, that is the quotients of Ki(V2)/Ki(V1b) and Ki(OT)/Ki(V1b) are in each case at least greater than 1.

20. A compound as claimed in claim 1, characterized in that it has a binding affinity Ki for the vasopressin V1b receptor subtype of less than 100 nm and simultaneous selectivities for the vasopressin V1b receptor subtype vis-à-vis the vasopressin V1a receptor subtype, the vasopressin V2 receptor subtype and the oxytocin (OT) receptor, that is the quotients of Ki(V1a)/Ki(V1b), Ki(V2)/Ki(V1b) and Ki(OT)/Ki(V1b) are in each case at least greater than 1.

21. A medicament comprising at least one compound as claimed in claim 1 and at least one pharmaceutically acceptable carrier.

22. A compound as claimed in claim 1 for use as medicament.

23. A method for the treatment and/or prophylaxis of at least one vasopressin-dependent comprising administering an effective amount of at lease one compound of the general formula (I) a claimed in claim 1 to a patient in need of such treatment.

24. (canceled)

25. (canceled)

26. (canceled)

27. (canceled)

28. (canceled)

29. (canceled)

30. (canceled)

31. (canceled)

32. A method for the treatment and/or prophylaxis of at least one disorder selected from the group consisting of diabetes insipidus, nocturnal enuresis, incontinence, diseases in which blood coagulation disorders occur, and for delaying micturation in a patient, characterized in that an effective amount of at least one compound of the general formula (I) as claimed in claim 1 is administered to the patient.

33. A method for the treatment and/or prophylaxis of at least one disorder selected from the group consisting of hypertension, pulmonary hypertension, heart failure, myocardial infarction, coronary spasm, unstable angina, PTCA (percutaneous transluminal coronary angioplasty), ischemias of the heart, disorders of the renal systems, edemas, renal vasospasm, necrosis of the renal cortex, hyponatremia, hypokalemia, Schwartz-Bartter syndrome, disorders of the gastrointestinal tract, gastritic vasospasm, hepatocirrhosis, gastric and intestinal ulcer, emesis, emesis occurring during chemotherapy, and travel sickness, in a patient, characterized in that an effective amount of at least one compound of the general formula (I) as claimed in claim 1 is administered to the patient.

34. A method for the treatment and/or prophylaxis of affective disorders in a patient, characterized in that an effective amount of at least one compound of the general formula (I) as claimed in claim 1 is administered to the patient.

35. A method for the treatment of anxiety disorders and/or stress-dependent anxiety disorders in a patient, characterized in that an effective amount of at least one compound of the general formula (I) as claimed in claim 1 is administered to the patient.

36. A method for the treatment of memory impairments and/or Alzheimer's disease in a patient, characterized in that an effective amount of at least one compound of the general formula (I) as claimed in claim 1 is administered to the patient.

37. A method for the treatment of psychoses and/or psychotic disorders in a patient, characterized in that an effective amount of at least one compound of the general formula (I) as claimed in claim 1 is administered to the patient.

38. A method for the treatment of Cushing's syndrome in a patient, characterized in that an effective amount of at least one compound of the general formula (I) as claimed in claim 1 is administered to the patient.

39. A method for the treatment of sleep disorders in a patient, characterized in that an effective amount of at least one compound of the general formula (I) as claimed in claim 1 is administered to the patient.

40. A method for preparing compounds of the general formula (I) as claimed in claim 1, characterized in that either an isatin derivative which is substituted in position 5 by a leaving or convertible radical which is suitable for replacement by or conversion into the cyano group, or a suitable 5-cyanoisatin derivative, is employed as starting material.

41. The method as claimed in claim 40, characterized in that the cyano group is introduced into position 5 of the oxindole ring by replacement and conversion in the first or the last step of the method or a step of the method in between.
